(19) United States
(12) Patent Application Publication
(10) Pub. No.: US 2025/0332183 A1
(43) Pub. Date: Oct. 30, 2025

(54) COMPOSITIONS AND METHODS FOR THE STIMULATION OF INTERMEDIATE MACROPHAGES AND TREATMENTS THEREWITH

(71) Applicant: AyuVis Research, Inc., Fort Worth, TX (US)

(72) Inventors: Suchismita Acharya, Grapevine, TX (US); Adam S. Dayoub, Arlington, TX (US); Santosh K. Panda, Maryland Heights, MO (US)

(21) Appl. No.: 19/194,559

(22) Filed: Apr. 30, 2025

Related U.S. Application Data
(60) Provisional application No. 63/640,552, filed on Apr. 30, 2024.

(51) Int. Cl. A61K 31/7034; A61K 9/00; A61K 39/39; A61K 45/06; A61P 37/06

(52) U.S. Cl. CPC A61K 31/7034; A61K 9/0019; A61K 39/39; A61K 45/06; A61P 37/06

(57) ABSTRACT

Provided herein are compositions and methods for method for inducing intermediate macrophages in a subject, the method comprising: administering to the subject a therapeutically effective amount of one or more compositions that comprise a compound of formula (I) or stereoisomer, enantiomer, tautomer or a pharmaceutically acceptable salt thereof:

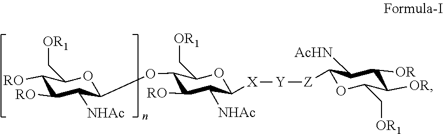

wherein the compound promotes monocyte differentiation into an antigen-presenting cell (APC)-specific intermediate macrophage lineage.

### CROSS-REFERENCE TO RELATED APPLICATIONS

This application claims priority to U.S. Provisional Application Ser. No. 63/640,552, filed Apr. 30, 2025, the entire contents of which are incorporated herein by reference.

### STATEMENT OF FEDERALLY FUNDED RESEARCH

This invention was made with government support under HD107857-01A1, awarded by the National Institutes of Health. The government has certain rights in the invention.

### TECHNICAL FIELD

The present disclosure relates in general to the field of compositions and methods for the stimulation of intermediate macrophages, and more particularly, to compositions and methods for the treatment of conditions in which intermediate macrophages provide an alternative immune response pathway that helps to prevent and treat bronchopulmonary dysplasia (BPD), retinopathy of prematurity and diseases related to immune imbalances.

### INCORPORATION-BY-REFERENCE OF MATERIALS FILED ON COMPACT DISC

None.

### BACKGROUND

Without limiting the scope of the invention, its background is described in connection with respiratory distress.

Bronchopulmonary Dysplasia (BPD) is a neonatal condition that occurs in infants born at <28 weeks of gestation and birth weights<1000 grams. The strongest risk factors for BPD are prematurity and low birth weight (Bhandari 2016). Secondary to premature birth, the babies have immature lungs. While affected infants can improve over time due to lung growth, they will suffer from significant morbidity in childhood, extending up to adulthood, due to neurodevelopmental impairment, asthma and emphysematous changes of the lung.

While many drugs have been tried to prevent/attenuate BPD (Bhandari 2014, Sahni and Bhandari 2020), no specific and effective treatment is available, and therefore this disease is still associated with high mortality and morbidity (Lui, Lee et al. 2019). Despite improved neonatal care, the number of BPD cases due to this condition have not decreased (Horbar, Edwards et al. 2017), secondary to increased survival of infants of lower gestational ages. Although exogenous surfactant is standard-of-care treatment for respiratory distress syndrome (RDS) in premature neonates, there is no effective prevention or treatment for BPD to date (Bhandari 2014).

Use of steroids as anti-inflammatory therapy is partially helpful in minimizing inflammation in BPD; however, in babies administered the drug (either antenatally and postnatally via parenteral or inhaled routes), the incidence of BPD is either not decreased or the risk of death and poor neurodevelopmental outcome outweighs the overall benefit. There have been no randomized clinical trials (RCTs) where inhaled budesonide has been used to treat ‘established BPD’ (Andrews 2020). In the largest RCT on inhaled budesonide (Bassler, Halliday et al. 2010), although there was a significant lowering of the incidence of BPD (Bassler 2017), there was no difference in neurodevelopmental outcomes (Bassler, Shinwell et al. 2018) and significantly increased mortality in the treatment group (Filippone, Nardo et al. 2019).

Retinopathy of Prematurity (ROP) affects more than 32,000 preterm babies/year worldwide (Hong et al., Retinopathy of prematurity: a review of epidemiology and current treatment strategies, Clin Exp Pediatr. 2022 March; 65(3): 115-126. Published online 2021 Oct. 12. doi: 10.3345/cep.2021.00773. ROP is among the most common causes of childhood blindness. Treatment options for preventing ROP progression include: (1) retinal ablation using cryotherapy; (2) laser therapy; and anti-vascular endothelial growth factor (anti-VEGF) treatments. Despite these advances, a need remains for compositions and methods for preventing and/or treating ROP, specifically, treatments that do not include the negative side effects of anti-VEGF treatments.

Despite these efforts, a need remains for novel compositions and methods to prevent or treat neonatal lung injury, bronchopulmonary dysplasia (BPD) and BPD-associated pulmonary hypertension (BPD-PH), and Retinopathy of Prematurity (ROP).

### SUMMARY

As embodied and broadly described herein, an aspect of the present disclosure relates to a method for inducing intermediate macrophages in a subject, the method comprising: administering to the subject a therapeutically effective amount of one or more compositions that comprise a compound of formula (I) or stereoisomer, enantiomer, tautomer or a pharmaceutically acceptable salt thereof:

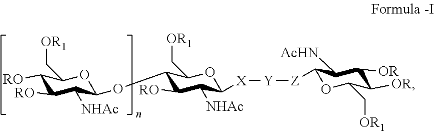

wherein n=0-5; X═NH, O, S, or CH₂; Y=Phenyl, a phenyl group substituted with at least one methyl, a phenyl group substituted with at least one nitro, a phenyl group substituted with at least one nitrogen, a phenyl group substituted with at least one boron, aryl, substituted aryl, heteroaryl, four to six membered cycloalkyl, four to six membered heterocycloalkyl; Z═NH, O, S, CH₂ or none; R═H, C(O)R₂, SO₂R₂; R₁═H, C(O)R₂, SO₂R₂; R₂=Ethyl, methyl, isopropyl, n-propyl, t-butyl, n-butyl, NH₂, NR₃R₄; R₃, R₄=ethyl, methyl, isopropyl, n-propyl, t-butyl, n-butyl, three to six membered cycloalkyl, wherein the compound promotes monocyte differentiation into an antigen-presenting cell (APC)-specific intermediate macrophage lineage. In one aspect, the composition at least one of: modifies polarization of macrophages to intermediate macrophages; modifies a balance between different subtypes of macrophages toward intermediate macrophages; induces differentiation of monocytes to intermediate macrophages; or induces phenotype switching from immature macrophages to intermediate macrophages. In another aspect, the compound is selected from:

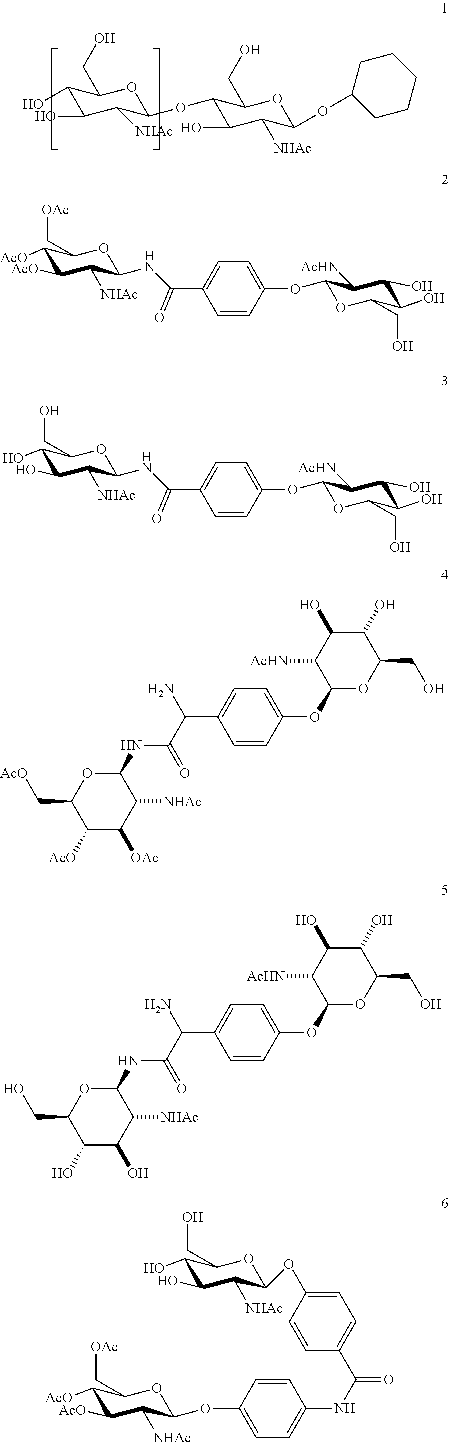

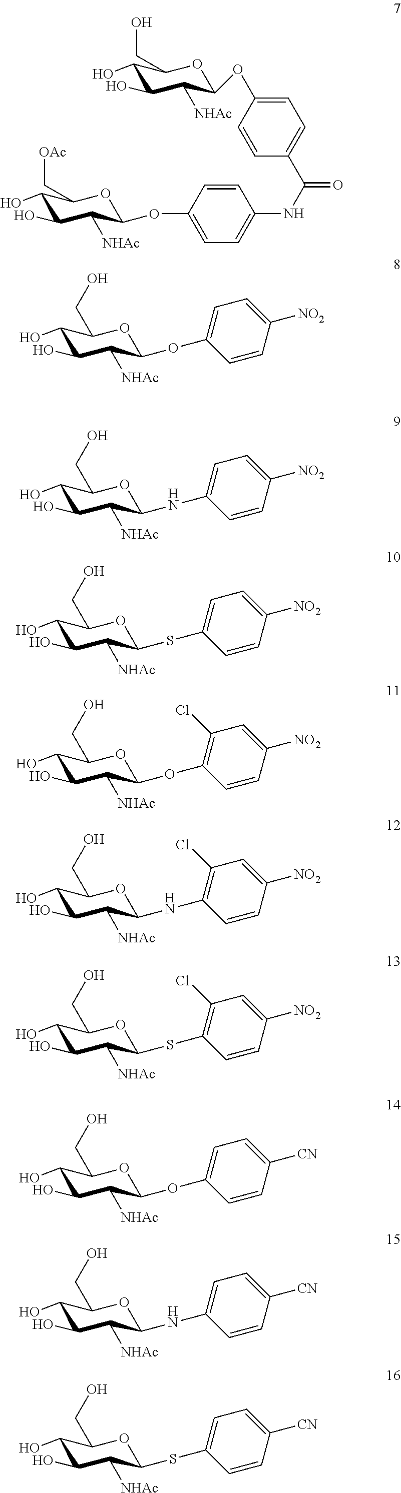

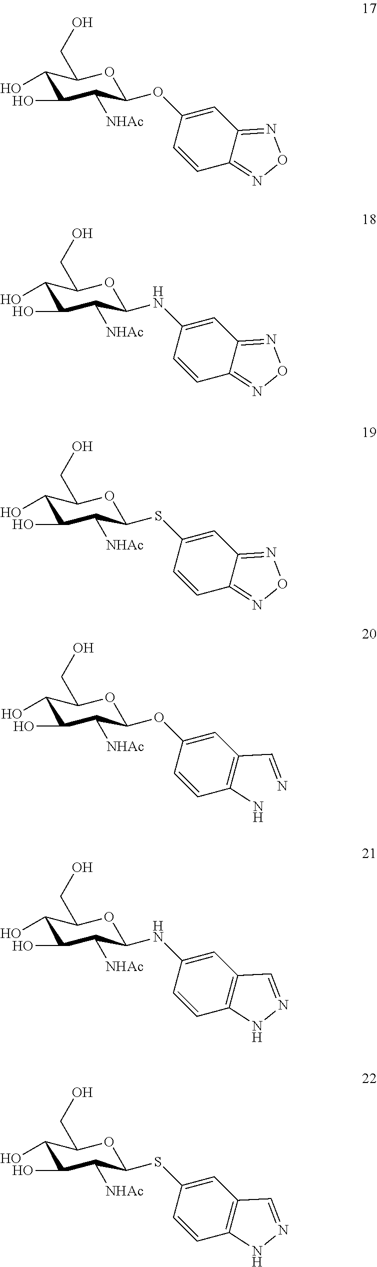

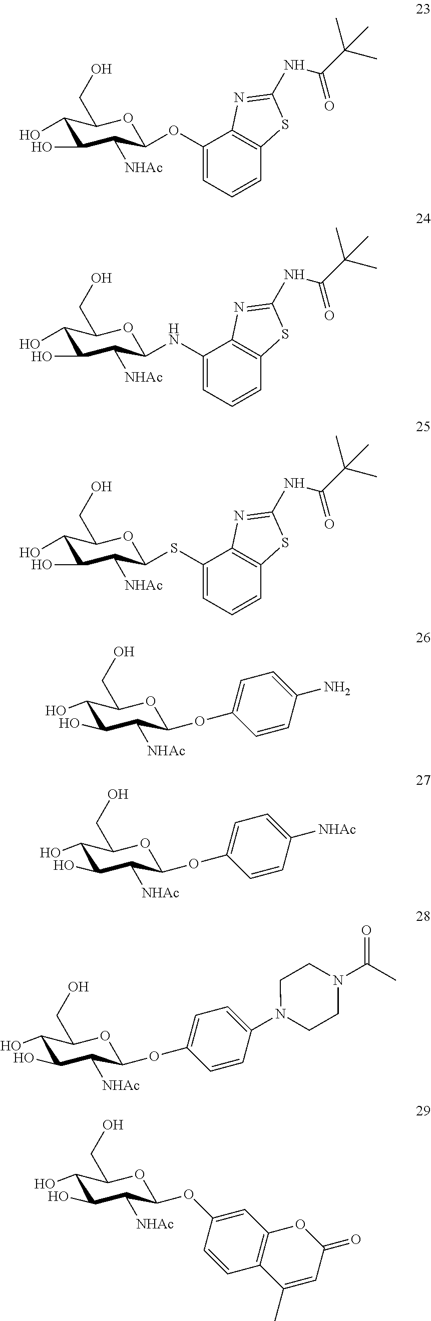

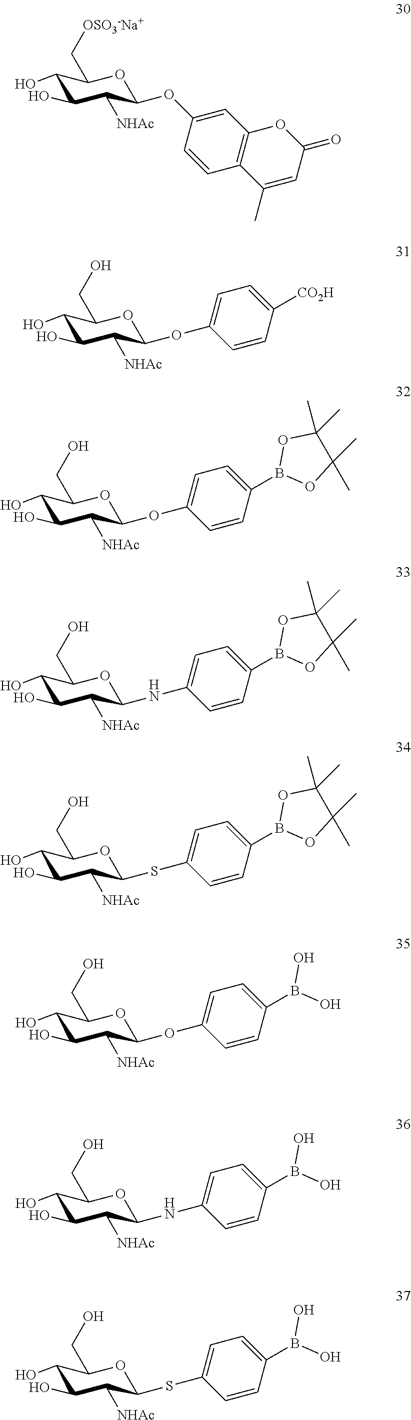

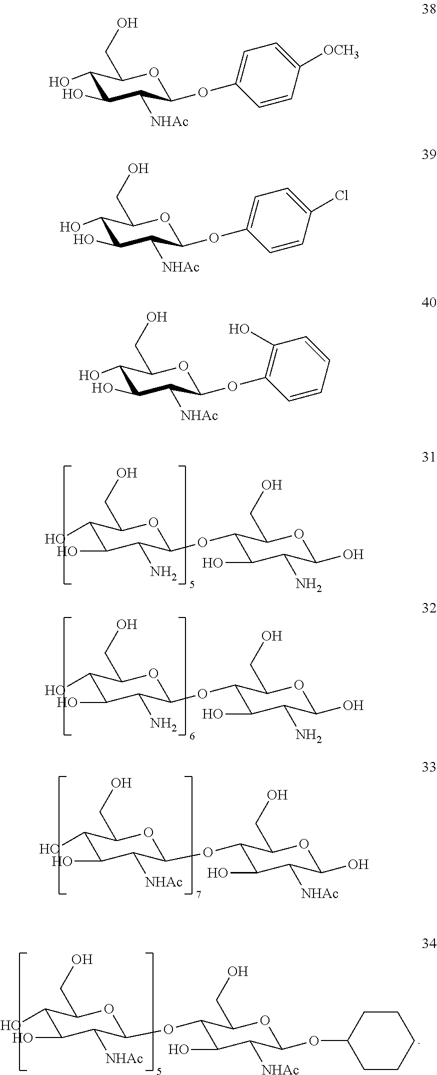

In another aspect, the compound is administered by pulmonary, alveolar, enteral, parenteral, intravenous, intraperitoneal, intramuscular, subcutaneous, topical, otic, ocular, intravitreal, or oral administration. In another aspect, the compound is combined with at least one active agent selected from: amylocaine, articaine, benzocaine, bupivacaine, chloroprocaine, dibucaine, etidocaine, levobupivacaine, lidocaine, mepivacaine, metabutoxycaine, piperocaine, prilocaine, procaine, proparacaine, ropivacaine, tetracaine, corticosteroids, bronchodilators, anticholinergics, vasodilators, diuretics, anti-hypertensive agents, acetazolamide, antibiotics, antivirals, or immunosuppressive drugs. In another aspect, the compound is

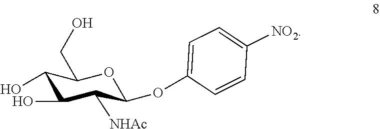

In another aspect, the compound is selected from at least one of:

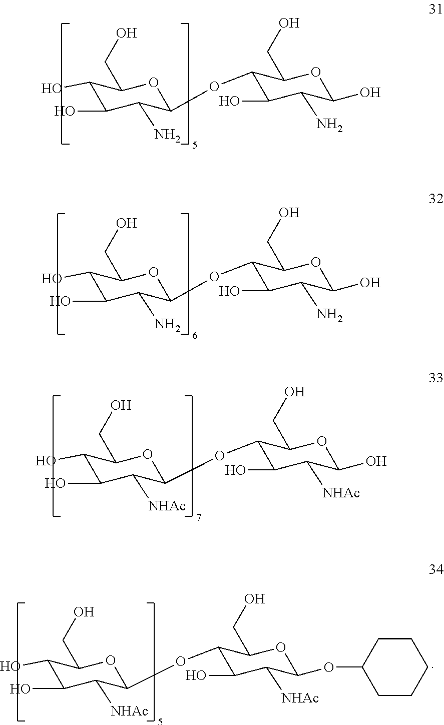

In another aspect, the intermediate monocytes are HLA-DR⁺/CD163⁺. In another aspect, the compound does not bind to or trigger VEGF receptor. In another aspect, the compound binds peripheral blood mononuclear cells at both TLR4 and CD163. In another aspect, the compound decreases inflammatory cytokines in cord blood cells and CD8+ T cells in retinopathy of prematurity (ROP). In another aspect, the compound overcomes immune cell tolerance and primes immunity for prevention or treatment of bronchopulmonary dysplasia. In another aspect, the compound has at least one of: anti-inflammatory, anti-angiogenic, or anti-fibrotic activities.

As embodied and broadly described herein, an aspect of the present disclosure relates to an adjuvant comprising: a compound of Formula I, or stereoisomer, enantiomer, tautomer or a pharmaceutically acceptable salt thereof:

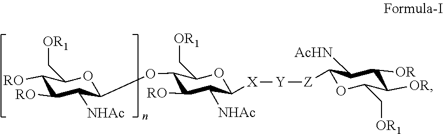

wherein n=0-5; X═NH, O, S, or CH₂; Y=Phenyl, a phenyl group substituted with at least one methyl, a phenyl group substituted with at least one nitro, a phenyl group substituted with at least one nitrogen, a phenyl group substituted with at least one boron, aryl, substituted aryl, heteroaryl, four to six membered cycloalkyl, four to six membered heterocycloalkyl; Z═NH, O, S, CH₂ or none; R═H, C(O)R₂, SO₂R₂; R₁═H, C(O)R₂, SO₂R₂; R₂=Ethyl, methyl, isopropyl, n-propyl, t-butyl, n-butyl, NH₂, NR₃R₄; R₃, R₄=ethyl, methyl, isopropyl, n-propyl, t-butyl, n-butyl, three to six membered cycloalkyl, wherein the compound promotes monocyte differentiation into an antigen-presenting cell (APC)-specific intermediate macrophage lineage. In another aspect, the composition at least one of: modifies polarization of macrophages to intermediate macrophages; modifies a balance between different subtypes of macrophages toward intermediate macrophages; induces differentiation of monocytes to intermediate macrophages; or induces phenotype switching from immature macrophages to intermediate macrophages. In another aspect, the compound is selected from:

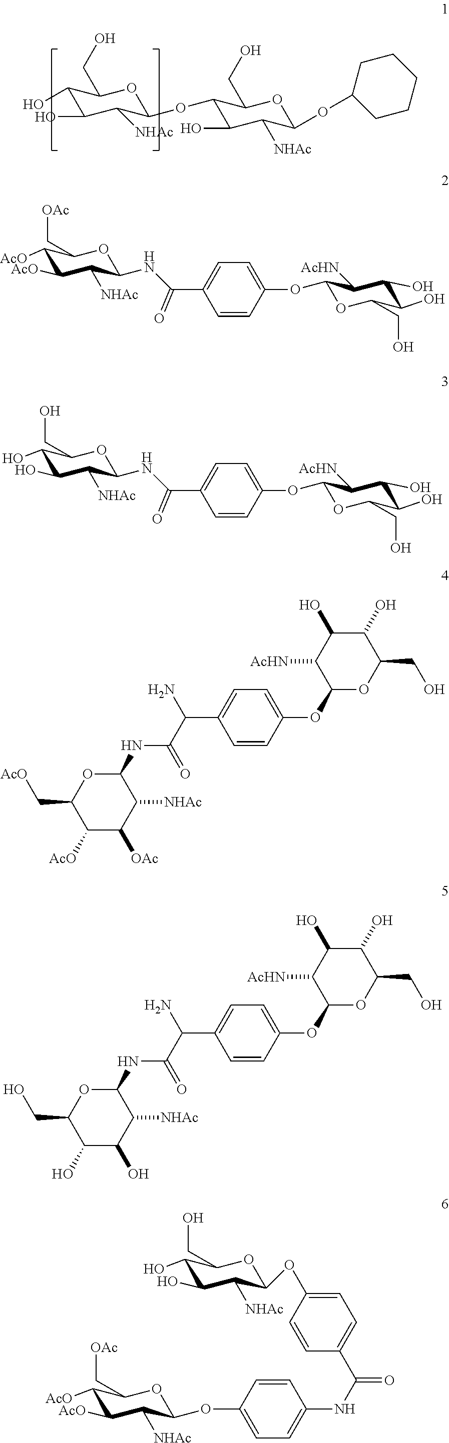

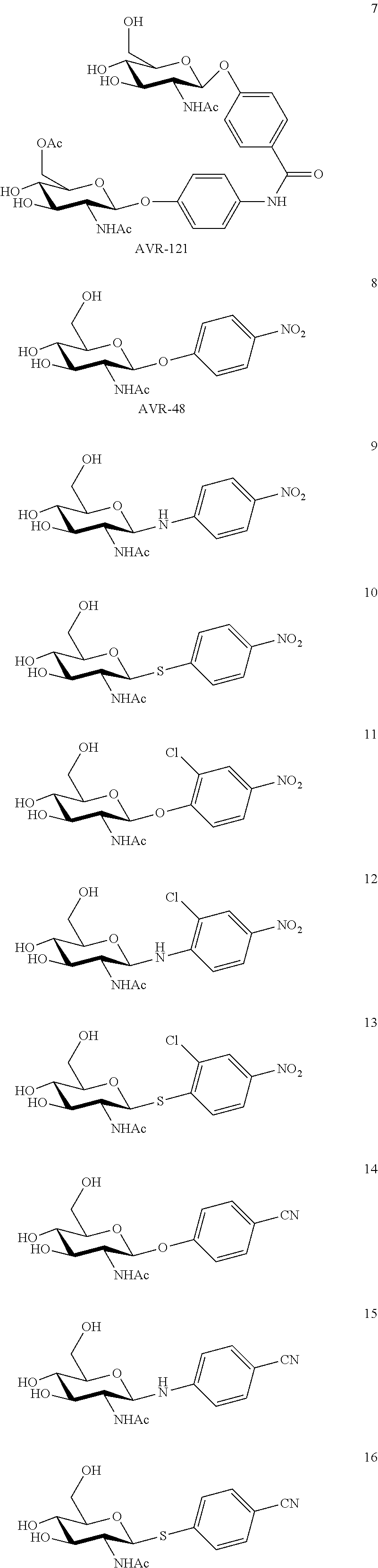

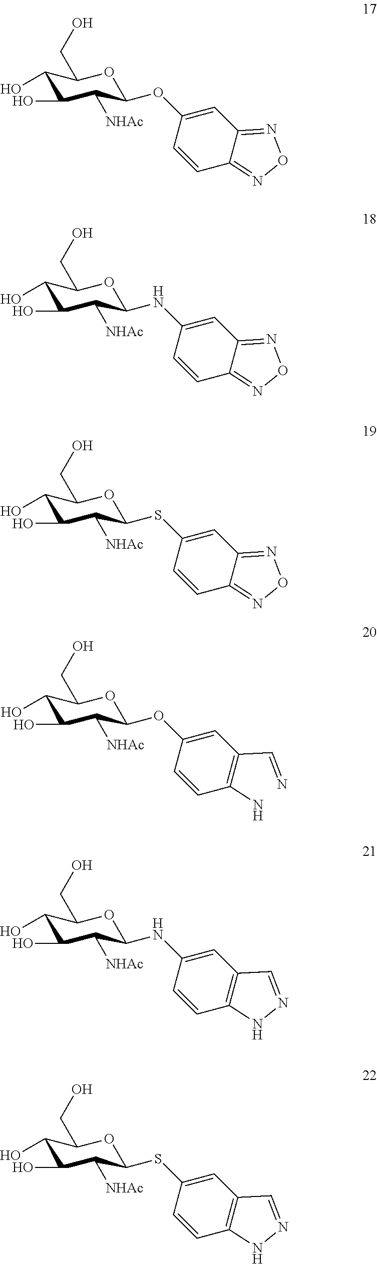

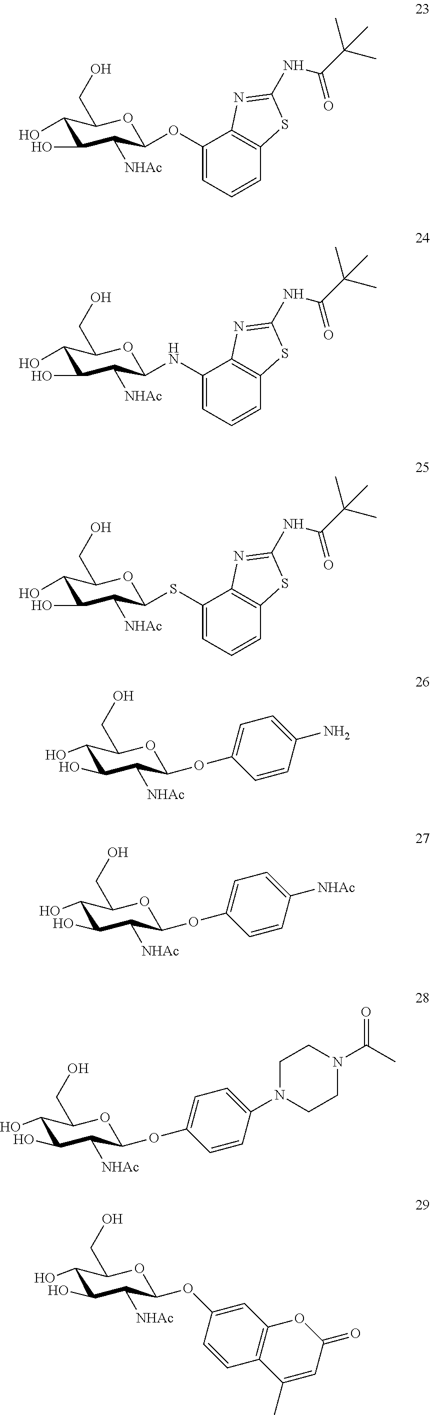

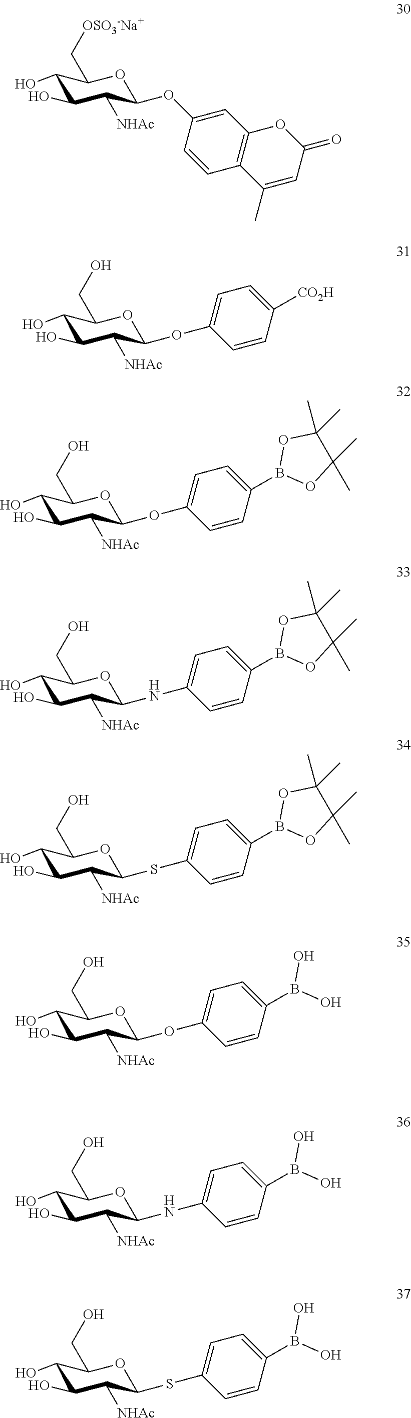

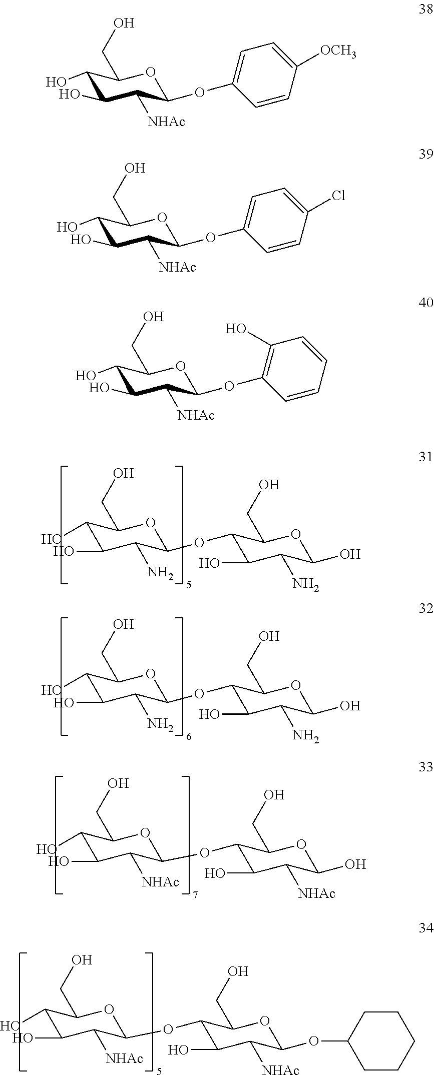

In one aspect, wherein the adjuvant induces an increase in intermediate macrophages, B cells, T cells, and antigen presenting cells. In another aspect, the adjuvant induces an increase in at least one of CD38+/CD27+ Plasma blasts; CD19+ B cells; CD4+ T-helper cells; CD8+ T cells; or IgG. In another aspect, the compound is administered by pulmonary, alveolar, enteral, parenteral, intravenous, intraperitoneal, intramuscular, subcutaneous, topical, otic, ocular, intravitreal, or oral administration. In another aspect, the compound is combined with at least one active agent selected from: amylocaine, articaine, benzocaine, bupivacaine, chloroprocaine, dibucaine, etidocaine, levobupivacaine, lidocaine, mepivacaine, metabutoxycaine, piperocaine, prilocaine, procaine, proparacaine, ropivacaine, tetracaine, corticosteroids, bronchodilators, anticholinergics, vasodilators, diuretics, anti-hypertensive agents, acetazolamide, antibiotics, antivirals, or immunosuppressive drugs. In another aspect, the compound is

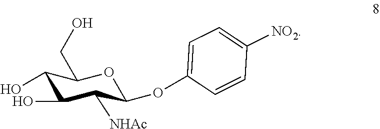

In another aspect, the compound is selected from at least one of

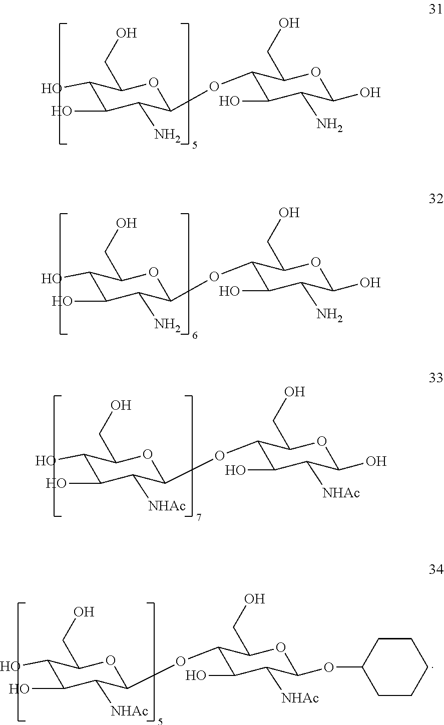

In another aspect, the intermediate monocytes are HLA-DR⁻/CD163⁺. In another aspect, the compound does not bind to or trigger VEGF receptor. In another aspect, the compound binds peripheral blood mononuclear cells at both TLR4 and CD163. In another aspect, the compound decreases inflammatory cytokines in cord blood cells and CD8+ T cells in retinopathy of prematurity (ROP). In another aspect, the compound overcomes immune cell tolerance and primes immunity for prevention or treatment of bronchopulmonary dysplasia. In another aspect, the compound has at least one of: anti-inflammatory, anti-angiogenic, or anti-fibrotic activities.

As embodied and broadly described herein, an aspect of the present disclosure relates to a method for preventing or treating inflammatory diseases, conditions, or symptoms, the method comprising administering to a subject a prophylactically or therapeutically effective amount of a composition containing one or more pharmaceutically acceptable carriers and a compound of Formula I, or stereoisomer, enantiomer, tautomer or a pharmaceutically acceptable salt thereof:

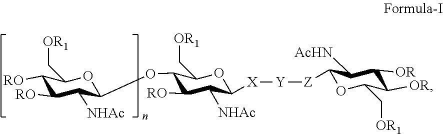

wherein n=0-5; X═NH, O, S, or CH₂; Y=Phenyl, a phenyl group substituted with at least one methyl, a phenyl group substituted with at least one nitro, a phenyl group substituted with at least one nitrogen, a phenyl group substituted with at least one boron, aryl, substituted aryl, heteroaryl, four to six membered cycloalkyl, four to six membered heterocycloalkyl; Z═NH, O, S, CH₂ or none; R═H, C(O)R₂, SO₂R₂; R₁═H, C(O)R₂, SO₂R₂; R₂=Ethyl, methyl, isopropyl, n-propyl, t-butyl, n-butyl, NH₂, NR₃R₄; R₃, R₄=ethyl, methyl, isopropyl, n-propyl, t-butyl, n-butyl, three to six membered cycloalkyl, wherein the compound promotes monocyte differentiation into an antigen-presenting cell (APC)-specific intermediate macrophage lineage. In one aspect, the inflammatory disease, condition, or symptom is related to (a) decreased intermediate macrophages compared to normal condition and/or (b) decreased proportion and/or increased number of intermediate monocyte-derived macrophage compared to normal condition. In another aspect, the inflammatory disease, condition, or symptom is selected from the group consisting of single or multiple organ failure or dysfunction, bronchopulmonary dysplasia, retinopathy or prematurity, sepsis, cytokine storm, fever, neurological dysfunction or impairment, loss of taste or smell, cardiac dysfunction, pulmonary dysfunction, liver dysfunction, acute or chronic respiratory dysfunction, graft versus host disease (GVHD), cardiomyopathy, vasculitis, fibrosis, ophthalmic inflammation, dermatologic inflammation, gastrointestinal inflammation, tendinopathies, allergy, asthma, rheumatoid arthritis, glomerulonephritis, pancreatitis, hepatitis, non-alcoholic steatohepatitis (NASH), inflammatory arthritis, gout, multiple sclerosis, psoriasis, acute respiratory distress syndrome (ARDS), diabetic ulcers, non-healing wounds, nonalcoholic fatty liver disease (NAFLD), scleroderma, pulmonary arterial hypertension, scar tissues, atherosclerosis, vascular inflammation, neonatal hypoxia-ischemia brain injury, traumatic brain injury, ischemic stroke, hemorrhagic stroke, amyotrophic lateral sclerosis, neurodegenerative disease, lung infection, remote lung injury, chronic obstructive pulmonary disease, transfusion-induced lung injury, cisplatin-induced kidney injury, renal ischemia-reperfusion injury, renal transplantation, cardiac ischemia and infarction, cardiac transplantation, Crohn's and ulcerative colitis, terminal ileitis, alcoholic steatohepatitis, hepatotoxicity, liver infection, remote liver injury, lupus, autoimmune diseases associated with acute or chronic inflammation, and acute or chronic inflammation associated with viral, bacterial, or fungal infection.

### BRIEF DESCRIPTION OF THE DRAWINGS

For a more complete understanding of the features and advantages of the present invention, reference is now made to the detailed description of the invention along with the accompanying figures and in which:

FIGS. 1A and 1B include a schematic of the experimental protocol and plasma concentrations of AVR-48 following intravenous infusion of AVR-48 in preterm (PT) lambs. FIG. 1A) Time-dependent decrease in plasma concentration of AVR-48 (3.0 mg/kg) after the first dose and 15 minutes after the second dose (12.25 h). FIG. 1B) Plasma concentration of AVR-48, 15 min after each repeat dose (12, 24, 36, 48, 96, 108, 120, 132, 144 hr) for 7 days. n=3/group. GraphPad Prism v10.4. D: day; DOL: day of life; GA: gestational age.

FIGS. 2A and 2B shows the survival and growth of the former preterm (FPT) and term reference lambs for the late-stage study. FIG. 2A) Kaplan Meir survival graph shows that AVR-48 treatment statistically increased survival (80% vs 40%) in FPT group compared to FPT vehicle group. FIG. 2B) Body weight showed significant growth of the FPT AVR-48 and term reference lambs compared to the FPT vehicle control lambs. Term reference lambs were never ventilated. Data presented as mean±SD. *p<0.05 (Kaplan Meier, Log-rank test), **p<0.01 (two-way ANOVA). Dunnett's multiple comparisons test. GraphPad Prism v10.4.

FIGS. 3A to 3E show the daily oxygenation indices in preterm (PT) lambs for the early-stage study. FIGS. 3A-3E) AVR-48 (3.0 mg/kg, n=9) given to PT lambs significantly improved RSS, 01, A-a gradient, and S/F ratio daily through day of life 1-7 compared to the vehicle control PT lambs (n=10). Data presented as mean±SD. *p<0.05, **p<0.01, ***p<0.001, ****p<0.0001, Two-way ANOVA, Šídák's multiple comparisons test, GraphPad Prism v10.4. RSS, respiratory severity score (MAP×FiO₂); OI, oxygenation index; A-a, alveolar-arterial; S/F, (SpO₂/FiO₂).

FIGS. 4A to 4P shows the histopathology and lung quantitative histology of mechanically ventilated lamb lungs for the early-stage and late-stage studies. FIG. 4A-4D) Eary-stage study. Histopathology (H/E stained) of preterm (PT) lamb lungs after treatment with AVR-48 (3.0 mg/kg, n=3) or vehicle (n=3). Terminal respiratory units (TRU), shown at the same magnification in Panels A and B, have more delicate and lacier parenchyma in the AVR-48 PT lamb lung relative to the vehicle control PT lamb lung. The asterisks indicate the regions shown at higher magnification in Panels C and D. Those panels show that secondary septa (arrows) are longer, and the distal airspacewalls are thinner in the AVR-48 PT lamb lung relative to the vehicle control PT lamb lung. Quantitative histology showed that radial alveolar count (FIG. 4E) and secondary septal volume density (FIG. 4F) were significantly greater in the AVR-48 PT group compared to the vehicle control PT group. Conversely, distal airspace wall thickness (FIG. 4G) was significantly less in the AVR-48 PT group compared to the vehicle control PT group. FIG. 4H-4M) Late-stage study. Histopathology (H/E stained) shows better structural development of TRUs and parenchyma in the former preterm (FPT) AVR-48 lambs (3.0 mg/kg, n=4) relative to TRUs and parenchyma in the lung of the FPT vehicle control lambs (n=3). Lung architecture of the FPT AVR-48 lungs was similar to that of the term reference lamb lungs (n=6). Quantitative histology showed that radial alveolar count (FIG. 4N) was significantly greater in the FPT AVR-48 group compared to the vehicle control PT group. While statistical differences were not detected for secondary septal volume density (FIG. 4O) or distal airspace wall thickness (FIG. 4P), the group average for secondary septal volume density appeared higher for the FPT AVR-48 group relative to the FPT vehicle group. Conversely, the group average for distal airspace wall thickness appeared thinner for the FPT AVR-48 group relative to the FPT vehicle group. Term reference lambs were never ventilated. Data presented as mean±SD. *p<0.05, **p<0.01, unpaired Student's t-test, One-way ANOVA, Dunnett's multiple comparison test, GraphPad Prism v10.4.

FIGS. 5A to 5D show the respiratory system mechanics measured noninvasively by the forced oscillation technique at the end of the early-stage study (day of life 10). FIG. 5A-5C) Respiratory system resistance at the indicated applied frequencies was significantly lower for the AVR-48 preterm (PT) lambs (3.0 mg/kg, n=3) compared to the matched vehicle PT lambs (n=3). FIG. 5D) Airway reactance under the curve at zero (AX) (5-41 Hz) was significantly lower for the AVR-48 PT lambs compared to the vehicle PT lambs. Data are presented as mean±SD. *p<0.05, **p<0.01, ****p<0.0001, Student's t-test, GraphPad Prism v10.4.

FIGS. 6A to 6C show the respiratory system mechanics measured noninvasively by the forced oscillation technique at the end of the late-stage study (day of life 90). FIG. 6A) Upper airways baseline resistance was significantly lower for the FPT AVR-48 group (n=4) compared to the FPT vehicle group (n=3). Reactance was significantly less negative for the FPT AVR-48 group than the FPT vehicle group. Baseline resistance and reactance were the same (superimposed in the graph) between the FPT AVR-48 and the term reference (n=6) groups. No differences were detected at the higher frequencies. FIG. 6B) Upper airways resistance and reactance of the FPT lambs at 2 moC PNA after methacholine challenge (2 mg) were respectively significantly different for the FPT AVR-48 group compared to the FPT vehicle group. FIG. 6C) After albuterol nebulization, the FPT AVR-48 group had significantly lower resistance and less negative reactance at 7-20 Hz frequencies compared to FPT vehicle group. Term reference lambs were never ventilated. Data are presented as mean±SD. *p<0.05, **p<0.01, ***p<0.001, ****p<0.0001, two-way ANOVA, Tukey's multiple comparisons test, GraphPad Prism v10.4.

FIGS. 7A to 7D show the neurobehavioral outcomes for former preterm (FPT) and term reference lambs at the end of the late-stage study (2 moC PNA; day of life 90). FIG. 7A-7C) Curiosity and socialization outcomes. Time at a novel object (FIG. 7A) was significantly shorter for the FPT AVR-48 group (3.0 mg/kg/dose, n=4) compared to the FPT vehicle group (n=3), and the same as that for the term reference group (n=6). Time at the dull surface (FIG. 7B) was significantly shorter, whereas time as the reflective surface was significantly longer (FIG. 7C), for the FPT AVR-48 group compared to the FPT vehicle group. Time at both surfaces was the same for the FOT AVR-48 group and the term reference group. Time to navigate the maze to reach the milk bottle reward (FIG. 7D) was less (not significant) for the FPT AVR-48 group compared to the FPT vehicle group. Navigation time for the FOT AVR-48 group was similar to that for the term reference group. Term reference lambs were never ventilated. Data presented as an average of two 10-min trials/group±SD. *p<0.05. Student's unpaired t-test. GraphPad Prism v10.4.

FIGS. 8A to 8H. Cytokine profiles in preterm (PT) lamb plasma during the early-stage study (day of life 1-10) and former preterm (FPT) lamb lungs at the end of the late-stage study (day of life 90). FIG. 8A-8C) The plasma IL-1β, IL-6, and IL-10 concentrations in vehicle-treated (n=4-7) and AVR-48-treated (n=6) preterm lambs at different time points determined by ELISA. Each cytokine concentration was significantly higher at specific hours of life for the AVR-48 (3.0 mg/kg dose) group compared to the vehicle control PT group. FIG. 8D) The plasma soluble CD163 concentration was significantly lower at the specific hour of life shown for the AVR-48 group (n=3) compared to the vehicle control PT group (n=3). FIG. 8E-8H) The relative protein level for TLR4 was significantly lower for the FPT AVR-48 group (n=3) compared to the FPT vehicle control group (n=3). Group average TNF-α and IL-6 protein levels appeared less for the FPT AVR-48 group relative to the FPT vehicle control group (not significant). Group average IL-10 appeared higher for the FPT AVR-48 group relative to the FPT vehicle control group (not significant). The lowest lung tissue protein levels were detected for the term reference group, which was never ventilated. Data are presented as mean SD, *p≤0.05, Ordinary one-way ANOVA, Dunnett's multiple comparisons test, **p<0.01. Two-way ANOVA, Šídák's multiple comparisons test. GraphPad Prism v10.4. TLR4, toll-like receptor 4; TNF-α, tumor necrosis factor alpha.

FIGS. 9A TO 9L show the mRNA and protein levels of apoptosis and proliferation markers in PT and FPT lamb lung homogenates. The relative mRNA levels for p53, caspase-3, c-Myc, and TGF-β for the vehicle (n=3) and AVR-48 (3.0 mg/kg, n=4) treated PT lambs lungs assessed at day 10, using RT-PCR (FIG. 9A-9D). FIG. 9E-9F) The relative protein levels for cleaved caspase-3 and PCNA were normalized to control for the vehicle (n=3) and AVR-48 (3.0 mg/kg, n=4) treated PT lambs lungs assessed at day 10 using Western blot. The relative mRNA levels for p53, caspase-3, c-Myc, and TGF-β for the FPT vehicle (n=3), AVR-48 (3.0 mg/kg, n=4) treated FPT lambs and term reference (n=6) lamb lungs assessed at day 90, using RT-PCR (FIG. 9G-9J). The relative protein levels of cleaved caspase-3 and PCNA for the FPT vehicle (n=3), FPT AVR-48 (3.0 mg/kg, n=4) and term reference (n=6) lambs lungs assessed at day 90, using Western blot (FIG. 9K-9L). Data is presented as mean±SD, *p<0.05, Student's unpaired t-test for A-F, One-way ANOVA, Dunnett's multiple comparison test for J-N. GraphPad Prism v10.4.

FIG. 10 shows a schematic of a 7-day treatment of AVR-48 (3.0 mg/kg, twice daily) significantly improved the respiratory parameters, lung injury, and neurobehavioral outcome in the preterm lamb model of BPD compared to vehicle-treated PT lambs.

FIGS. 11A to 11D show that AVR-48 improved neurobehavioral outcomes in FPT lambs in the late-stage study at 1 moC PNA (day of life 60). Tests were for curiosity, socialization, learning, and memory. FIG. 11A-11C) Curiosity and socialization outcomes are time at a novel object, dull (non-reflective) surface, and mirror (reflective surface) for vehicle-treated (n=3), AVR-48 (3.0 mg/kg/dose, n=4), and term reference lambs (n=6). (FIG. 11A-11C). No differences were detected at 1 moC PNA among the groups. Learning and memory outcomes for the maze test, with a milk bottle reward (FIG. 11D). Term reference lambs were never ventilated. Results presented as an average of two 10-min trials/group±SD. One-way ANOVA, Dunnett's multiple comparison test, GraphPad Prism v10.4.

FIGS. 12A to 12N. mRNA and protein levels of surfactant proteins and permeability markers in PT and FPT lamb lung homogenates. In the early-stage study, at 10d, VEGF-A protein level was significantly lower in AVR-48 PT group than vehicle PT groups (FIG. 12F). Otherwise, no significant difference was detected between the two groups of PT lambs for other markers. For the late-stage study, at 90d, no significant difference was detected between FPT vehicle and FPT AVR-48 lamb lungs, or between those groups and the term reference lamb lungs for both mRNA expression and protein level for VEGF-A, VEGFR2, and SP-B. VEGF-R₂ protein level was significantly greater in the term reference group compared to both FPT groups (FIG. 12L). Data is presented as mean±SD, *p<0.05, Student's unpaired t-test for A-G, One-way ANOVA, Dunnett's multiple comparison test for H-N. GraphPad Prism v10.4.

FIGS. 13A and 13B show that AVR-48 binds to TLR4 and CD163. CBMCs treated with Biotinylated-AVR-48 [100 μM] exhibit a competitive & dose-dependent binding to TLR2 and 4 in (FIG. 15A) monocytes and (FIG. 15B) lymphocytes. In addition, AVR-48 exhibits a binding affinity to CD163 with both immune cell types. N=3 *p<0.05, **p<0.01, ***p<0.001, **** p<0.0001. Two-way ANOVA was used for statistical analysis.

FIGS. 14A to 14D show Western Blot analysis for TLR4 pathway and densitometry of (FIG. 14A-FIG. 14C) hPBMC and (FIG. 14D) CBMCs after treatment with AVR-48 [100 μM], LPS [50 ng/mL], and in combination indicates AVR-48 activates TLR4 pathway (hPBMC) via phospho-MyD88 and -TIRAP, with no activation via TRAM, and inhibiting the phosphorylation of the NF-kB pathway.

FIGS. 15A TO 15F show the AVR-48 cytokine profile in CBMCs. Cells treated with AVR-48 [100 μM], LPS [50 ng/mL] and AVR-48+LPS show a decrease (˜2-4-fold) in cytokine signal in (FIG. 15A) TNF-α, (FIG. 15B) IL-1, (FIG. 15C) IL-10, (FIG. 15D) IL-12, (FIG. 15E) TL-6, and (FIG. 15F) TL-8, and indicates a reduced anti-inflammatory response as compared to LPS treated cells solo. N=3, One-way ANOVA was used for analysis.

FIGS. 16A and 16B show that AVR-48 overcomes LPS-challenge in CBMCs. Cells dosed with AVR-48 [100 μM], LPS [50 ng/mL], and in combination show (FIG. 16A) pre-treating CBMC with AVR-48 followed by LPS insult shows an inhibited IL-1β LPS-derived profile. N=3, One-way ANOVA was used for analysis. In addition, (FIG. 16B) LPS-tolerated CBMCs show that AVR-48 can also overcome tolerance via TNF-α. N=3, Two-way ANOVA was used for statistical analysis.

FIG. 17 show the AVR-48 induced intermediate macrophage lineage. CBMCs treated with AVR-48 [100 μM] and LPS [50 ng/mL] exhibit a preference for intermediate macrophage lineage (HLA-DR⁻/CD163⁺) which suggests AVR-48 unique antigen-presenting cell (APC) capacity and action. N=3 *P<0.05, Two-way ANOVA was used for statistical analysis.

FIG. 18 is a graph that shows the effect of AVR-48 in peripheral blood monuclear cells (PBMCs) as a percent of parent cells—3, 7 and 10 days after treatment.

FIG. 19 is a summary of the different stages of retinopathy of prematurity.

FIGS. 20A to 20D show that AVR-123 decreased vaso-obliteration and angiogenesis in mouse model of OIR. (FIG. 20A) AVR-123 was dosed once-a-day eye drop (AVR-123NP, 1% nanosuspension) or IP injection (10 mg/kg) for 5 days (P7-P12 and P12-P17 respectively). At P18 mouse pups are sacrificed and retina flat mounts were prepared. (FIG. 20B) AVR-123 decreased area of Vaso obliteration via IP injection (FIG. 20C) via eye drop. (FIG. 20D) The retina was stained with isolectin to visualize the blood vessels. Image J, N=3-5 retina.

FIGS. 21A to 21B show the effect of AVR-123 treatment on mouse splenic immune cells from the OIR mice. (FIG. 21A) The splenic immune cell populations from OIR mice injected IP with AVR-123 (P7-P12) were collected at P18, and the cells (n=3 mouse spleens) were stained with immune cell-specific antibodies and analyzed via flow cytometry. (FIGS. 21B, 21C) The splenic immune cell populations from OIR mice treated with AVR-123 (ED) during P12-P17. Treatment with AVR-123 reduced populations of macrophages (F4/80+CF11b+), CD8+ T cells (CD3+CD8+), neutrophils (Ly6G+CD11b+), and dendritic cells (CD11c+MHCII+) in mouse pup spleens compared to the hyperoxia mouse group. n=3. * p<0.05, t-test.

FIG. 22 shows the effect of AVR-123 treatment on mouse splenic immune cells from the OIR mice. The splenic immune cell populations from OIR mice injected IP with AVR-123 (P7-P12) were collected at P18, and the cells (n=3 mouse spleens) were stained with immune cell-specific antibodies and analyzed via flow cytometry. The splenic immune cell populations from OIR mice treated with AVR-123 (ED) during P12-P17. Treatment with AVR-123 reduced populations of macrophages (F4/80+CF11b+), CD8+ T cells (CD3+CD8+), neutrophils (Ly6G+CD11b+), and dendritic cells (CD11c+MHCII+) in mouse pup spleens compared to the hyperoxia mouse group. n=3. * p<0.05, t-test.

FIGS. 23A to 23G show that AVR-123's anti-inflammatory activity is via downregulating NFkB. CBMCs were treated with AVR-123 [100 μM], LPS [50 ng/mL], or AVR-123+LPS for 24 h. AVR-123+LPS showed a decrease (˜2-4-fold) in cytokine concentrations for (FIG. 25A) TNF-α, (FIG. 25B) IL-10, (FIG. 25C) IL-12, (FIG. 25D) IL-6, (FIG. 25E) IL-8, and (FIG. 25F) IL-10, compared to LPS treated cells. G) AVR-123 decreases phosphorylated NFkB alone and when combined with LPS in CBMCs via western blot analysis. N=3, One-way ANOVA.

FIGS. 24A and 24B show that LPS tolerance is reversed with AVR-123 treatment. FIG. 24A) Low-dose LPS (1 ng/mL) pre-treatment for 48 h, followed by high-dose (50 ng/mL) LPS for another 24 h in CBMCs showed a lower level of TNF-α, an indication of tolerance, where the addition of AVR-123 significantly increased TNF-α. FIG. 24B) Pretreatment of AVR-48 for 48 h followed by LPS treatment for an additional 24 h showed a significantly higher TNF-α level than the control. N=3 *p<0.05, **p<0.01, ***p<0.001. One-way ANOVA and t tests.

FIGS. 25A to 25H show that pretreatment of AVR-48 in mice activated the innate and adaptive immune response in the presence of LPS to demonstrate an immune modulation motif (FIG. 25A) The AVR-48 group increased M1 and (FIG. 25B) Mint macrophages as an innate immune action-reaction (FIG. 25C), whereas (FIG. 25D) M2 macrophages were not produced. AVR-48 stimulated innate and adaptive immune response via (FIG. 25E) TNF-α increase and (FIG. 25F) CD3+, (FIG. 25G) CD4+, and (FIG. 25H) CD8+ T-cells, a necessary step in adaptive response and immune resolution. *p<0.05. **p<0.01. ***p<0.001; N=4, One-way ANOVA was used for analysis.

FIG. 26A to 26C show the AVR-48 induced immune profile after AVR-48 pre-dosing and dosing before LPS and MPLA insult for 7 days. (FIG. 26A) AVR-48 outperformed MPLA in CD4+T-T-cells and CD27− T-cells production. (FIG. 27B) Compared to MPLA, AVR-48 stimulated more TEMRE effector T-cell production. (FIG. 27C) AVR-48 perturbed CD8+ IFN-g+T-memory and helper cell recruitment in the presence of influenza-A peptide. N=3, *p<0.05, **p<0.01, ***p<0.001; One-way ANOVA.

FIG. 27 shows the study design for in vivo testing. Study Design: N=4/per group (Balbc/6J, 6-7 months old). Group-1: (OVA+CFA) 1^{st} dose; (OVA+IFA) 2^{nd} dose; OVA-3rd dose; Group 2: OVA+CFA+AVR-48 1^{st} dose; OVA+IFA+AVR-48 2^{nd} dose; OVA+AVR-48 3^{rd} dose; and Group 3: OVA+IFA+AVR48 1^{st} dose; OVA+IFA+AVR-48 2^{nd} dose; OVA+AVR-48 3^{rd} dose.

FIGS. 28A and 28B show that AVR-48 is more efficacious than Complete Freund's Adjuvant regarding plasma cell formation and IgG induction. Vaccination of AVR-48 along with Ovalbumin±CFA/IFA. Increased CD138+/CD38+/CD27+ Plasma cells in mouse spleen (FIG. 28A), and increased IgG in serum (FIG. 28B).

FIG. 29 shows the Mouse Model of Influenza Peptide Vaccination study design.

FIGS. 30A to 30E show that AVR-48 is more efficacious in improving humoral immunity than the currently used adjuvant MPLA in a mouse Flu vaccination study. Vaccination of AVR-48 alone or with Flu-A peptide increased: (FIG. 30A) CD38+/CD27+ plasma blasts; (FIG. 30B) CD19+ B cells; (FIG. 30C) CD4+ T-helper cells; (FIG. 30D) CD8+ T cells in the mouse spleen; and (FIG. 30E) increased IgG in mouse serum.

### DETAILED DESCRIPTION

While the making and using of various embodiments of the present disclosure are discussed in detail below, it should be appreciated that the present disclosure provides many applicable inventive concepts that can be embodied in a wide variety of specific contexts. The specific embodiments discussed herein are merely illustrative of specific ways to make and use the invention and do not delimit the scope of the invention.

To facilitate the understanding of this disclosure, a number of terms are defined below. Terms defined herein have meanings as commonly understood by a person of ordinary skill in the areas relevant to the present disclosure. Terms such as “a”, “an” and “the” are not intended to refer to only a singular entity, but include the general class of which a specific example may be used for illustration. The terminology herein is used to describe specific embodiments of the disclosure, but their usage does not delimit the disclosure, except as outlined in the claims.

The present invention combines surfactants isolated from lungs, such as bovine and porcine lungs (e.g., from pups or calves), with a bioactive molecule of Formula I:

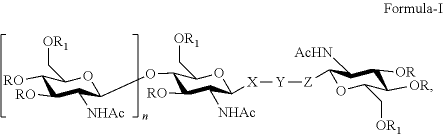

where n=0-5; X═NH, O, S, or CH₂; Y=Phenyl, or a phenyl group substituted with at least one methyl, a phenyl group substituted with at least one nitro, a phenyl group substituted with at least one nitrogen, a phenyl group substituted with at least one boron, or aryl, substituted aryl, heteroaryl, four to six membered cycloalkyl, four to six membered heterocycloalkyl; R═H, C(O)R₂, SO₂R₂; R₁ ═H, C(O)R₂, SO₂R₂; R₂=Ethyl, methyl, isopropyl, n-propyl, t-butyl, n-butyl, NH₂, NR₃R₄; R₃, R₄=Ethyl, methyl, isopropyl, n-propyl, t-butyl, n-butyl, three to six membered cycloalkyl and Z═NH, O, S, CH₂, or none. In one aspect, an amount of the compound is varied or selected to either inhibit or activate the immune response. In one aspect, the compound has the formula:

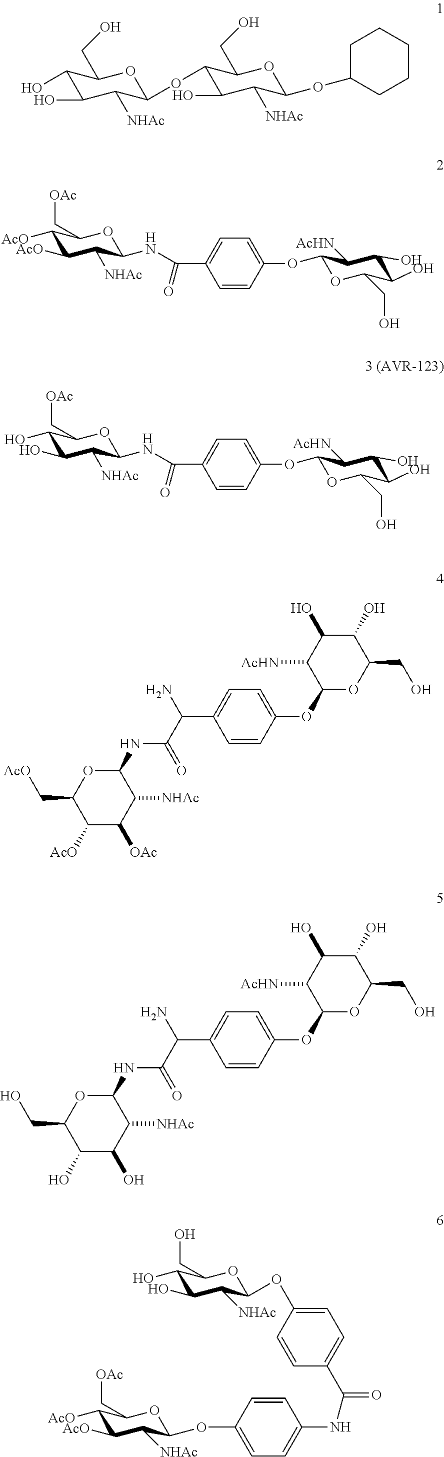

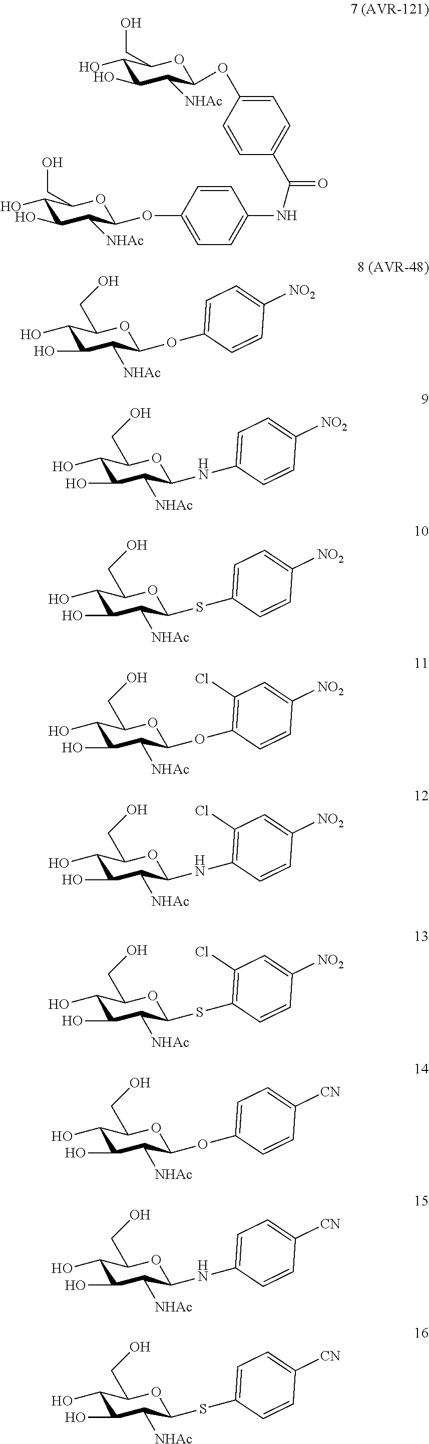

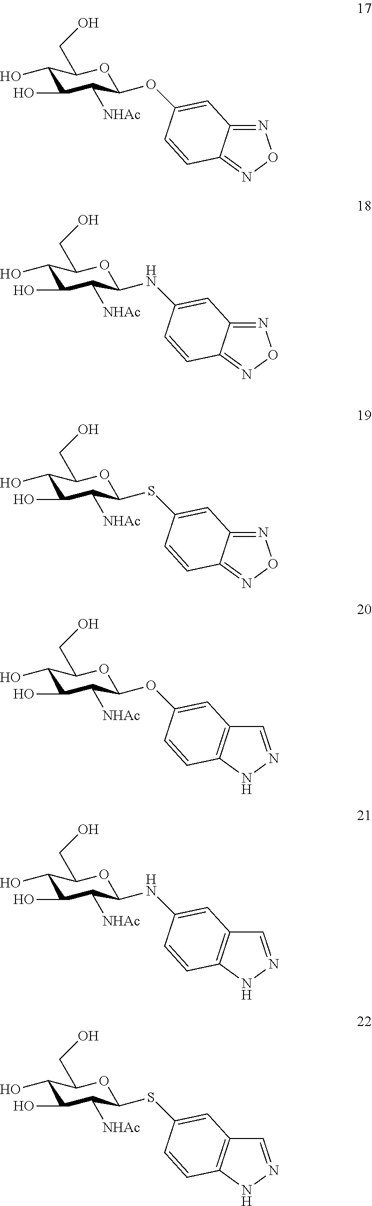

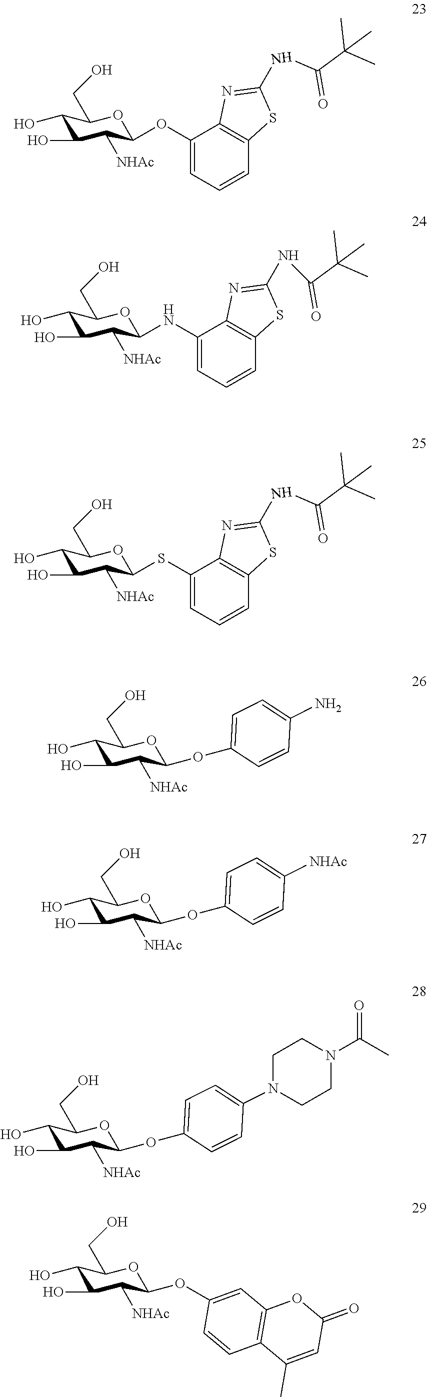

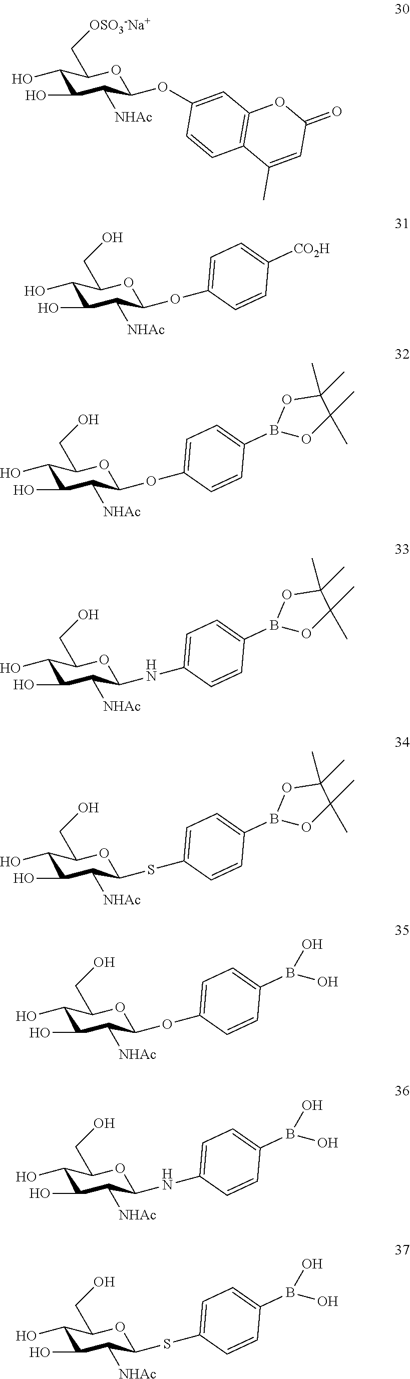

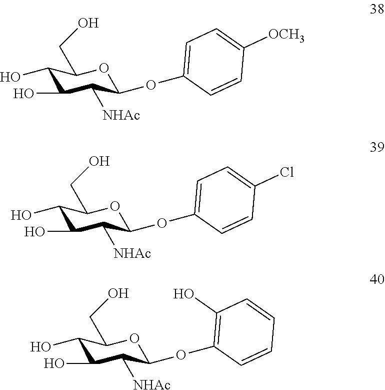

The compounds of the present invention find particular uses in the delivery of particles of low density and large size for drug delivery to the pulmonary system. Biodegradable particles have been developed for the controlled-release and delivery of compounds, such as those disclosed herein. Langer, R., Science, 249: 1527-1533 (1990).

The respiratory tract encompasses the upper airways, including the oropharynx and larynx, followed by the lower airways, which include the trachea followed by bifurcations into the bronchi and bronchioli. The upper and lower airways are called the conducting airways. The terminal bronchioli then divide into respiratory bronchiole, which then lead to the ultimate respiratory zone, the alveoli, or deep lung. The present invention can be formulated for delivery to any part of the respiratory tract, e.g., Gonda, I. “Aerosols for delivery of therapeutic and diagnostic agents to the respiratory tract,” in Critical Reviews in Therapeutic Drug Carrier Systems 6:273-313, 1990, relevant portions incorporated herein by reference. On one non-limiting example, the deep lung or alveoli are the primary target of inhaled therapeutic aerosols for systemic drug delivery of the present invention.

Inhaled aerosols have been used for the treatment of local lung disorders including asthma and cystic fibrosis and have potential for the systemic delivery of the compounds of the present invention. Pulmonary drug delivery strategies present many difficulties for the delivery of macromolecules, including: excessive loss of inhaled drug in the oropharyngeal cavity (often exceeding 80%), poor control over the site of deposition, irreproducibility of therapeutic results owing to variations in breathing patterns, the often too-rapid absorption of drug potentially resulting in local toxic effects, and phagocytosis by lung macrophages.

Considerable attention has been devoted to the design of therapeutic aerosol inhalers to improve the efficiency of inhalation therapies and the design of dry powder aerosol surface texture. The present inventors have recognized that the need to avoid particle aggregation, a phenomenon that diminishes considerably the efficiency of inhalation therapies owing to particle aggregation, is required for efficient, consistent deep lung delivery.

In one example for a formulation for pulmonary delivery, particles containing the active compound(s) of the present invention may be used with local and systemic inhalation therapies to provide controlled release of the therapeutic agent. The particles containing the active compound(s) permit slow release from a therapeutic aerosol and prolong the residence of an administered drug in the airways or acini, and diminish the rate of drug appearance in the bloodstream. Due to the decrease in use and increase in dosage consistency, patient compliance increases.

The human lungs can remove or rapidly degrade hydrolytically cleavable deposited aerosols over periods ranging from minutes to hours. In the upper airways, ciliated epithelia contribute to the “mucociliary escalator” by which particles are swept from the airways toward the mouth. It is well known that, in the deep lung, alveolar macrophages are capable of phagocytosing particles soon after their deposition. The particles containing the active compound(s) provided herein permit for an effective dry-powder inhalation therapy for both short- and long-term release of therapeutics, either for local or systemic delivery, with minimum aggregation. The increased particle size consistency is expected to decrease the particles' clearance by the lung's natural mechanisms until drugs have been effectively delivered.

PLGA encapsulated nanosuspension with extended drug release profile Nanoparticle formulation. Nanoparticle formulation can be carried out through a single or double emulsion technique. For example, for a single emulsion technique, 10 mg of compounds Or was dissolved in 3 ml of chloroform containing 100 mg of PLGA to form an oil phase. This solution was then added dropwise into 20 ml of 5% PVA solution (water phase) and emulsified at 50 W for 5 minutes to form the compound loaded nanoparticles. The final emulsion was stirred overnight to allow solvent evaporation. The nanoparticles were washed and collected by ultracentrifugation and lyophilized before use.

For example in a double emulsion technique, 30 mg of poly(D,L-lactide-co-glycolide) (PLGA) were dissolved in 1 mL of chloroform at 4° C. Concurrently, 2 mL of a 2% w/v poly(vinyl alcohol) (PVA)/distilled deionized water solution was formed. Upon solubilization of the PVA in water, 1 mL of ethanol or methanol was added as a non-solvent to the PVA solution. The active compound was then added to the PVA/ethanol solution at a concentration of 1 mM and stirred. A stock solution of active agent, e.g., 10 mg/ml, is formed by the dissolution of curcumin into water under alkaline conditions using, e.g., 0.5 M NaOH. The active agent is added to the PLGA/Chloroform solution at concentrations of 0.5, 1.0, and 2.0 mg/mL per 150 microliters of aqueous volume. Formation of the primary emulsion is done by vortexing the active agent-PLGA/chloroform solution for 20 seconds, followed by tip sonication at 55 W for 1 minute on a Branson Sonifier model W-350 (Branson, Danbury, CN). The primary emulsion is then added to a BS3/PVA/ethanol solution to initiate formation of the secondary emulsion. Completion of the secondary emulsion is done through vortexing for 20 seconds and tip sonication at 55 W for 2 minutes. Stabile activated nanoparticles are then aliquoted into 1.5 mL Eppendorf tubes and centrifuged for 5 minutes at 18,000 g. The chloroform and residual PVA supernatant were aspirated off and particles were resuspended by tip sonication in, e.g., 1 mL of phosphate buffered saline (PBS) pH 7.2. Following resuspension, nanoparticles were placed at −80° C. for 1 hour and lyophilized overnight. Lyophilization can be carried out in an ATR FD 3.0 system (ATR Inc, St. Louis, MO) under a vacuum of 250 μT. After lyophilization nanoparticles are stored at 4° C. Upon use nanoparticles were weighed into Eppendorf tubes and resuspended in 1 mL of PBS pH 7.4.

In some embodiments, the compounds of the present disclosure are incorporated into parenteral formulations. The term parenteral as used herein includes subcutaneous, intravenous, intramuscular, and intra-arterial injections with a variety of infusion techniques. Intra-arterial and intravenous injection as used herein includes administration through catheters. Preferred for certain indications are methods of administration that allow rapid access to the tissue or organ being treated, such as intravenous injections for the treatment of endotoxemia or sepsis.

The compounds of the present disclosure will be administered in dosages which will provide suitable inhibition or activation of TLRs of the target cells; generally, these dosages are, preferably between 0.25-50 mg/patient, or from 1.0-100 mg/patient or from 5.0-200 mg/patient or from 100-500 mg/patient, more preferably, between 0.25-50 mg/patient and most preferably, between 1.0-100 mg/patient. The dosages are preferably once a day for 28 days, more preferably twice a day for 14 days or most preferably 3 times a day for 7 days.

Pharmaceutical compositions containing the active ingredient may be in any form suitable for the intended method of administration. Techniques and compositions for making useful dosage forms using the present invention are described in one or more of the following references: Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, New York, 1990; Katzung, ed., Basic and Clinical Pharmacology, Ninth Edition, McGraw Hill, 2007; Goodman and Gilman, eds., The Pharmacological Basis of Therapeutics, Tenth Edition, McGraw Hill, 2001; Remington's Pharmaceutical Sciences, 20th Ed., Lippincott Williams & Wilkins., 2000, and updates thereto; Martindale, The Extra Pharmacopoeia, Thirty-Second Edition (The Pharmaceutical Press, London, 1999); all of which are incorporated by reference, and the like, relevant portions incorporated herein by reference.

The present invention includes compositions and methods for making and generating aerosols for delivery of the active agents described herein at the specific doses. In one embodiment, the compounds are formulation to be aerosolized with an aerosol-generating device. A typical embodiment of this invention includes a liquid composition having predetermined physical and chemical properties that facilitate forming an aerosol of the formulation. Such formulations typically include three or four basic parameters, such as, (i) the active ingredient; (ii) a liquid carrier for the active ingredient; (iii) an aerosol property adjusting material; and optionally, (iv) at least one excipient. The combination of these components provides a therapeutic composition having enhanced properties for delivery to a user by generating an inhalable aerosol for pulmonary delivery.

Aqueous suspensions of the compounds of the present invention contain the active materials in admixture with excipients suitable for the manufacture of aqueous suspensions. Such excipients include a suspending agent, such as sodium carboxymethylcellulose, methylcellulose, hydroxypropylmethyl cellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadeaethyleneoxycetanol), a condensation product of ethylene oxide with a partial ester derived from a fatty acid and a hexitol anhydride (e.g., polyoxyethylene sorbitan mono-oleate). The aqueous suspension may also contain one or more preservative such as ethyl of n-propyl p-hydroxybenzoate.

The pharmaceutical compositions of the invention can be in the form of a sterile injectable preparation, such as a sterile injectable aqueous or oleaginous suspension. This suspension may be formulated according to the known art using those suitable dispersing or wetting agents and suspending agents, which have been mentioned above. The sterile injectable preparation may also be a sterile injectable solution or suspension in a non-toxic parenteral-acceptable diluent or solvent, such as a solution in 1,3-butanediol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose, any bland fixed oil may be employed including synthetic mono- or diglycerides. In addition, fatty acids such as oleic acid may likewise be used in the preparation of injectables.

In some embodiments the formulation comprises PLA or PLGA microparticles and may be further mixed with Na₂HPO₄, hydroxypropyl methylcellulose, polysorbate 80, sodium chloride, and/or edetate disodium.

Formulations suitable for parenteral administration include aqueous and non-aqueous isotonic sterile injection solutions which may contain anti-oxidants, buffers, bacteriostats and solutes which render the formation isotonic with the blood of the intended recipient; and aqueous and non-aqueous sterile suspensions which may include suspending agents and thickening agents. The formulations may be presented in unit-dose or multi-dose sealed containers, for example, ampules and vials, and may be stored in a freeze-dried (lyophilized) condition requiring only the addition of the sterile liquid carrier, for example water for injections, immediately prior to use. Extemporaneous injection solutions and suspensions may be prepared from sterile powders of the kind previously described.

Ophthalmic Drop (Eyedrops) and Ointment Formulations. Eye drops are typically aqueous or aqueous and oil solutions, emulsions, or suspensions of one or more active ingredients, which may contain preservatives if stored in multiuse packaging. Eye formulations are sterile and isotonic. The optimum pH for eye drops equals that of tear fluid, about pH 7.4. The stability of active ingredient and the tissue tolerance to the preparation will dictate the requirement for buffer. If the pH exceeds pH 4 to 8, the formulation may cause discomfort and/or irritation (e.g., burning, stinging), and drug bioavailability can decrease because of increased tearing. Components may include buffers such as citrate, phosphate or borate, preservatives such as mercuric salts, polyquaternium, zinc salts, and benzalkonium salts, lubricants such as glycerin, surfactants such as tyloxapol, and polysorbates, viscosity modifiers such as hydrophilic polymers of high molecular weight which do not diffuse through biological membranes and which form three-dimensional networks in the water such as polyvinyl alcohol, poloxamers, hyaluronic acid, carbomers, and polysaccharides, such as, cellulose derivatives, gellan gum, and xanthan gum, carriers such as polyoxyethylene-polyoxypropylene block copolymer (poloxamer 407), mucoadhesive hydrophilic polymers such as macromolecular hydrocolloids with many hydrophilic groups (carboxyl, hydroxyl, amide, and sulfate), polyacrylic acid, sodium carboxymethyl cellulose, and chitosan, as well as lectins, cross-linked polyacrylic acids which exhibit mucoadhesive properties, such as carbomer and carbopol, and cyclodextrins. Ointments usually contain solid or semisolid hydrocarbon base of melting or softening point close to body temperature. Carriers may include liposomes built of phosphatidylcholine, stearylamine, and various amounts of cholesterol or lecithin and α-L-dipalmitoylphosphatidylcholine, SolulanC24, a derivative of lanolin, which is a mixture of ethoxylated cholesterol (ether of cholesterol and polyethylene glycol), ethoxylated fatty alcohols (ether of cetyl alcohol and polyethylene glycol), and polyamidoamine (PAMAM).

The eye drop dosage form is easy to apply but suffers from the inherent drawback that most of the instilled volume is eliminated from the pre-corneal area resulting in a bioavailability ranging from 1-10% of total administrated dose. The poor bioavailability and rapid pre-corneal elimination of drugs given in eye drops is mainly due to conjunctival absorption, rapid solution drainage by gravity, induced lachrymation, blinking reflex, low corneal permeability and normal tear turnover. Because of poor ocular bioavailability, many ocular drugs are applied in high concentrations. This cause both ocular and systemic side-effects, which is often related to high peak drug concentrations in the eye and in systemic circulation. The frequent periodic instillations of eye drops are necessary to maintain a continuous sustained therapeutic drug level. This may result in a massive and unpredictable dose of medication.

Suspension types of pharmaceutical dosage forms are formulated with relatively water insoluble drugs to avoid the intolerably high toxicity created by saturated solutions of water-soluble drugs. However, the rate of drug release from the suspension is dependent upon the rate of dissolution of the drug particles in the medium, which varies constantly in its composition with the constant inflow and outflow of lachrymal fluid. In order to overcome the limitations of (a) short residence time, (b) pulsed dosing of drug, (c) frequent instillation, (d) large drainage factor, other delivery methods may be employed, including ophthalmic inserts.

Ophthalmic Inserts. Ophthalmic inserts are sterile, thin, multilayered, drug-impregnated, solid or semisolid devices placed into cul-de-sac or conjunctival sac and whose size and shape are especially designed for ophthalmic application. They are composed of a polymeric support containing drug(s) incorporated as dispersion or a solution in the polymeric matrix. The main objective of an ophthalmic insert is to increase the contact time between the preparation and the conjunctival tissue to ensure a sustained release to the ocular surface. In comparison with the traditional ophthalmic preparation i.e., eye drops, solid ophthalmic inserts may offer advantages such as (a) increased contact time and bioavailability, (b) prolonged drug release and thus better efficacy, (c) reduction of adverse effects, and (d) reduction of the number of administrations and thus better patient compliance. The foreign-body sensation of an insert presents a challenge. Discomfort can lead to poor-patient compliance, excessive lachrymation that accompanies irritation, dilutes the drug and causes reduction in its concentration. A properly designed ocular insert will minimize the sensation caused by its insertion and wear. Desired criteria for a controlled release ocular insert include: (1) Ease of handling and insertion; (2) Lack of expulsion during wear; (3) Reproducibility of release kinetics (e.g., zero-order drug delivery); (4) Applicability to variety of drugs; (5) Non-interference with vision and oxygen permeability; (6) Sterility; (7) Stability; and/or (8) Ease of manufacture Classification of patented ocular inserts.

Diffusion-based inserts. Diffusion inserts are composed of a central reservoir of drug enclosed in specially designed semi-permeable or micro porous membranes, which allow the drug to diffuse the reservoir at a precisely determined rate. The drug release from such a system is controlled by the lachrymal fluid permeating through the membrane until a sufficient internal pressure is reached to drive the drug out of the reservoir. The drug delivery rate is controlled by diffusion through the membrane. The central reservoir may be composed of glycerin, ethylene glycol, propylene glycol, water, methyl cellulose mixed with water, sodium alginate, poly (vinylpyrrolidone) or polyoxyethylene stearate. Membranes may be composed of polycarbonates, polyvinyl chloride, polysulfones, cellulose esters, crosslinked poly (ethyl oxide), cross-linked polyvinylpyrrolidone, and cross-linked polyvinyl alcohol (Rathore and Nema, 2009). Copolymers for minidiscs may include α-ω-bis(4-methacryloxy)-butyl poly(dimethylsiloxane) and poly(hydroxyethyl methacrylate.

Osmotic inserts. Osmotic inserts are generally composed of a central part surrounded by a peripheral part. The central part may be composed of a single reservoir or two distinct compartments. In first case, it is composed of a drug with or without an additional osmotic solute dispersed through a polymeric matrix, so that the drug is surrounded by the polymer as discrete small deposits. In the second case, the drug and the osmotic solutes are placed in two separate compartments, the drug reservoir being surrounded by an elastic impermeable membrane and the osmotic solute reservoir by a semi-permeable membrane. The second peripheral part of osmotic inserts comprises in all cases a covering film made of an insoluble semi-permeable polymer. Tear fluid diffuses into peripheral deposits through the semi-permeable polymeric membrane, wets and induces dissolution. The solubilized deposits generate a hydrostatic pressure against the polymer matrix causing its rupture under the form of apertures. Drug is then released through these apertures from the deposits near the surface of the device, which is against the eye, by the sole hydrostatic pressure. This corresponds to the osmotic part characterized by zero order drug release profile. Water permeable matrices may include ethylene-vinyl esters, copolymers, plasticized polyvinyl chloride (PVC), polyethylene and cross-linked polyvinylpyrrolidone (PVP). Semi-permeable membranes may include cellulose acetate derivatives, ethyl vinyl acetate (EVA), or polyesters of acrylic and methacrylic acids (Eudragit @). Osmotic agents may include inorganic components such as magnesium sulfate, sodium chloride, potassium phosphate, sodium carbonate and sodium sulfate, or organic components such as calcium lactate, magnesium succinate, tartaric acid, sorbitol, mannitol, glucose or sucrose (Rathore and Nema, 2009).

Soft contact lenses as inserts. Soft contact lenses are composed of covalently crosslinked hydrophilic or hydrophobic polymers that form a three-dimensional network or matrix capable of retaining water, aqueous solution or solid components. A hydrophilic contact lens may be soaked in a drug solution, thereby absorbing the drug, but does not give precise delivery as compared to some other non-soluble ophthalmic inserts. The drug release from soft contact lenses is generally very rapid at the beginning, declining exponentially with time. The release rate can be decreased by incorporating the drug homogeneously during the manufacture or by adding a hydrophobic component (Rathore and Nema, 2009).

Soluble inserts. Soluble inserts are the oldest type of ophthalmic insert. They offer the great advantage of being entirely soluble so that they do not need to be removed from their site of application thus limiting the interventions to insertion only. They may contain natural polymers like collagen or synthetic or semi-synthetic polymers. Therapeutic agent is absorbed by soaking the insert in a solution containing the drug, drying and rehydrating before use on the eye. The amount of drug loaded depends on the amount of binding agent, concentration of the drug solution and soaking duration. Soluble synthetic polymers may include cellulose derivatives such as hydroxypropyl cellulose, methylcellulose, hydroxyethyl cellulose and hydroxypropyl cellulose, polyvinyl alcohol or ethylene vinyl acetate copolymer. Additives may include plasticizers such as polyethylene glycol, glycerin, propylene glycol, enteric coated polymers such as cellulose acetate phthalate, hydroxypropyl methylcellulose and phthalate, complexing agents such as polyvinyl pyrrolidone, and bioadhesives such as polyacrylic acids (Rathore and Nema, 2009).

Biodegradable ophthalmic inserts. Biodegradable or bioerodible inserts are composed of material homogeneous dispersion of a drug included or not into a hydrophobic coating which is substantially impermeable to the drug. They are made of the so-called biodegradable polymers. Successful biodegradable materials for ophthalmic use are the poly (orthoesters) and poly(orthocarbonates). The release of the drug from such a system is the consequence of the contact of the device with the tear fluid inducing a superficial diversion of the matrix (Rathore and Nema, 2009). Biodegradable inserts may contain cellulose derivatives, like hydroxypropyl methylcellulose (HPMC), hydroxyethyl cellulose (HEC), sodium carboxymethyl cellulose, ethyl cellulose, acrylates, like, polyacrylic acid and its cross-linked forms, Carbopol or Carbomer, chitosan, starch, for example, drum-dried waxy maize starch, and excipients, such as mannitol, sodium stearyl fumarate and magnesium stearate, polymers such as poly(alkyl cyanoacrylate), polylactic acid, poly(epsilon-caprolactone), poly(lactic-co-glycolic acid), chitosan, gelatin, sodium alginate and albumin (Barnaowski et al., 2014).

It will be understood, however, that the specific dose level for any particular patient will depend on a variety of factors including the activity of the specific compound employed; the age, body weight, general health, and sex of the individual being treated; the time and route of administration; the rate of excretion; other drugs which have previously been administered; and the severity of the particular disease undergoing therapy.

In some embodiments the compositions of the present disclosure also contain from about 80% to about 99.5%, preferably from about 90 or 95% to about 98.5% of a compatible non-aqueous pharmaceutically acceptable topical vehicle. Some vehicles are described in U.S. Pat. No. 4,621,075, which is incorporated herein for this disclosure. Although it is preferred that these vehicles be free of water, the compositions of the present invention may contain up to about 5% water without significant adverse effects on the formation of the desired gels. These non-aqueous vehicle components are also well-known in the pharmaceutical arts, and they include (but are not limited to) short chain alcohols and ketones and emollients, such as hydrocarbon oils and waxes, lanolin and lanolin derivatives, silicone oils, monoglyceride, diglyceride, and triglyceride esters, fatty alcohols, alkyl and alkenyl esters of fatty acids, alkyl and alkenyl diesters of dicarboxylic acids, polyhydric alcohols and their ether and ester derivatives; wax esters and beeswax derivatives. Preferred vehicles incorporate methanol, ethanol, n-propanol, isopropanol, butanol, polypropylene glycol, polyethylene glycol and mixtures of these components. Particularly preferred vehicles include ethanol, n-propanol and butanol, especially ethanol. These preferred solvents may also be combined with other components, such as diisopropyl sebacate, isopropyl myristate, methyl laurate, silicone, glycerine and mixtures of these components, to provide non-aqueous vehicles which are also useful in the present invention. Of these additional components, diisopropyl sebacate is especially useful. In fact, preferred vehicles include mixtures of ethanol and diisopropyl sebacate in ratios, by weight, of from about 4:1 to about 1:4. Preferred vehicles contain from about 15% to about 35% diisopropyl sebacate and from about 65% to about 85% ethanol.

Compositions of the present invention may additionally contain, at their art-established usage levels, compatible adjunct components conventionally used in the formulation of topical pharmaceutical compositions. These adjunct components may include, but are not limited to, pharmaceutically-active materials (such as supplementary antimicrobial or anti-inflammatory ingredients, e.g., steroids) or ingredients used to enhance the formulation itself (such as excipients, dyes, perfumes, skin penetration enhancers, stabilizers, preservatives, and antioxidants). Examples of such agents include the pharmaceutically-acceptable acidic carboxy polymers, such as the Carbopol compounds commercially available from B. F. Goodrich Chemicals, Cleveland, Ohio.

In one embodiment, the compounds of the present invention may be formulated into a cream, lotion or gel packaged in a common trigger spray container will be firmly adhered to the area of interest as a regular cream does after it is sprayed out from the container. This is described in WO 98/51273, which is incorporated herein by reference. Accordingly, in one aspect, the present disclosure provides a pharmaceutical that can be incorporated into a non-aerosol spray composition for topical application, which comprises the compounds as described herein alone or in combination. The compounds are present in an amount in the range of 0.1% to 20% or in some embodiments from 1 to 15% by weight, or in some embodiments from 2 to 10% by weight of cream, lotion or gel. The compounds of the present invention can be incorporated into a neutral hydrophilic matrix cream, lotion or gel. In a first embodiment, the cream or lotion matrix for topical application is characterized by polyoxyethylene alkyl ethers. In a second embodiment, the gel is characterized by high molecular weight polymer of cross-linked acrylic acid. Polyoxyethylene alkyl ethers are non-ionic surfactants widely used in pharmaceutical topical formulations and cosmetics primarily as emulsifying agents for water-in-oil and oil-in-water emulsions. It is characterized in this invention as a base for non-aerosol trigger sprayable cream or lotion. Cross-linked acrylic acid polymer (Carbomer) employed to form the gel is another object of this invention.

A particularly suitable base for non-aerosol spray is therefore a cream or lotion containing from 1 to 25% of polyoxyethylene alkyl ethers, 3 to 40% of humectant and 0.1 to 1% of preservative or preservatives and the balance to 100% being purified water. Aptly the polyoxyethylene alkyl ether can be one or any combination selected from the group consisting of polyoxyl 20 cetostearyl ether (Atlas G-3713), poloxyl 2 cetyl ether (ceteth-2), poloxyl 10 cetyl ether (ceteth-10), poloxyl 20 cetyl ether (ceteth-20), poloxyl 4 lauryl cetyl ether (laureth-4), poloxyl 23 lauryl cetyl ether (laureth-23), poloxyl 2 oleyl ether (oleth-2), poloxyl 10 oleyl ether (oleth-10), poloxyl 20 oleyl ether (oleth-20), poloxyl 2 stearyl ether (steareth-2), poloxyl 10 stearyl ether (steareth-10), poloxyl 20 stearyl ether (steareth-20), and poloxyl 100 stearyl ether (steareth-100). Suitable humectant can be one or any combination selected from the group consisting of propylene glycol, polyethylene glycol, sorbitol or glycerine. Suitable preservative is one or any combination selected from the group consisting of methylparaben, propylparaben, benzyl alcohol, benzoic acid, sodium benzoate, sorbic acid and its salt or phenylethyl alcohol.

Another suitable base for non-aerosol spray is a gel containing from 0.1 to 2.0% of Carbomer, 0.1 to 1% of alkaline solution, 3 to 40% of humectant and 0.1 to 1% of preservative or preservative as and the balance to 100% being purified water. Aptly the Carbomer can be one or any combination selected from the group consisting of Carbomer 934, Carbomer 940 or Carbomer 941. The suitable humectant, preservative and purified water for the gel are same as that in the case or cream or lotion. Other sprayable formulations are described in US Pre-Grant Publication US2005/00255048, which is expressly incorporated herein by reference.

### Example 1. Efficacy Study in a Mechanically Ventilated Pre-Term Lamb BPD Model

To increase clinical translatability of AVR-48 (Compound 8/AVR-48), the drug exposure to lung and plasma was evaluated in a large-animal model that mimics preterm human infants and demonstrate efficacy of intravenous AVR-48 to prevent the development of BPD phenotype. The unique preterm lamb model, developed in Dr. Kurt Albertine's lab at the University of Utah, emulates the clinical setting for preterm human infants with respiratory failure related to premature birth before the lungs are mature enough to support extra-uterine life. Both the preterm lamb model and preterm human infants are whole-organism physiological beings that have the setting of preterm birth and mechanical ventilation with oxygen-rich gas because of respiratory failure related to lung structural and functional immaturity, including surfactant deficiency. Ventilation support with oxygen-rich gas for days, weeks, or months is associated with further co-morbidities of the brain, liver, distal ileum, and kidney injury, and inadequate nutrition and poor postnatal growth. This preterm lamb model for BPD continues to provide mechanistic insights during the evolution of BPD, development of multiple-organ dysfunction, and long-term structural and functional impairments. (Dahl, Veneroni et al. 2021) The present disclosure shows the efficacy of AVR-48 in preventing key aspects of BPD, using the inventors' established preterm lamb model of evolving human BPD, including prematurity, mechanical ventilation, and exposure to supplemental oxygen.¹⁴

For dose-optimization, preterm (PT) lambs were delivered at ˜128d (d; saccular stage of lung development; ˜85% gestation) after maternal antenatal steroids exposure. The efficacious dose of AVR-48 of 3.0 mg/kg (intravenously (iv)) was given every 12 h from postnatal day of life (DOL) 1-7. After delivery and surfactant administration, PT lambs were maintained on invasive mechanical ventilation (IMV) for 7d followed by 3d noninvasive respiratory support. For the early-stage study, outcomes were determined for PT lambs over DOL1-10. For the late-stage study, outcomes were determined for former preterm (FPT) lambs (vehicle, AVR-48 treated) that were weaned from respiratory support after 10d and were maintained in the lamb intensive care unit for a further 80d. Outcomes included functional, structural, molecular, and neurobehavioral assessments. Term reference lambs that were never ventilated were used as developmental references for the FPT lambs. The results show that AVR-48 improved lung respiratory gas exchange, alveolar formation, respiratory system mechanics, and neurobehavioral and cognitive outcomes compared to vehicle controls.

AVR-48 was synthesized by AyuVis Research (Fort Worth, TX, USA) in >95% purity.

PT lamb model and methods for all outcome measures.

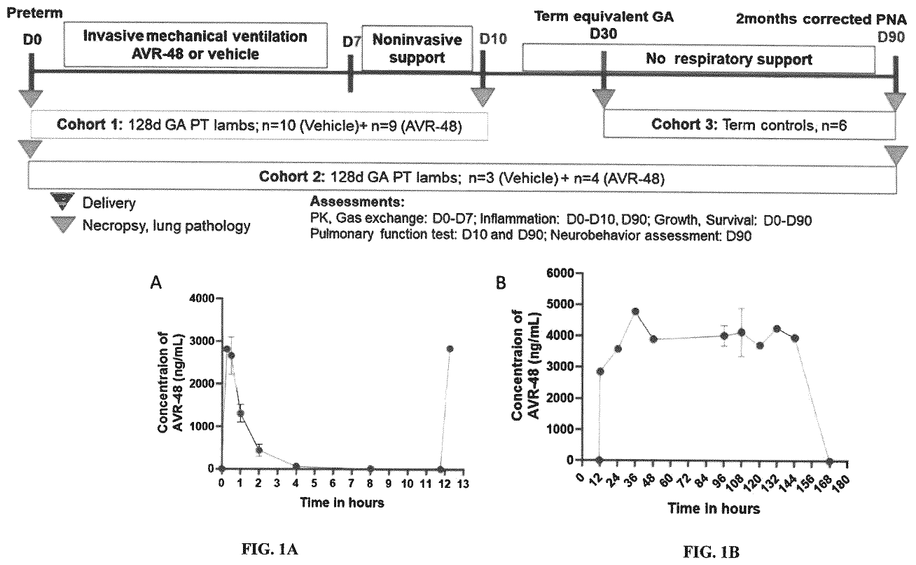

Study timeline. FIGS. 1A and 1B show the timeline for the two-endpoint study using two groups of PT lambs. The first endpoint used PT lambs managed by IMV for 7d, followed by extubation to noninvasive respiratory support (NRS) for up to 3d of life (DOL7-10). At that time, terminal tissues were collected. The second endpoint used the same management protocol, followed by weaning the lambs from respiratory support and having them live in the lamb intensive care unit as former PT (FPT) lambs for 2 months corrected postnatal age (3 months chronological age, DOL 90). The rationale for the 2-month corrected postnatal age for FPT lambs is when sheep are weaned from milk, equivalent to about 12-18 months for humans. Methods for the FPT lamb model have been reported.^{14,17}

PT lamb model and methods for all outcome measures. Surgical preparation and management of lamb groups. Delivery and clinical management methods for PT lambs (6-8) and FPT lambs (3, 4). Briefly, time-pregnant ewes that had single or twin fetuses at ˜128d (˜85%) gestation (term ˜150d gestation) were used (main FIGS. 1A and 1B). The fetuses were exposed to antenatal steroids (dexamethasone) at ˜48 h and ˜24 h before Cesarean-section (exit-procedure) delivery. An endotracheal tube was inserted, and catheters were placed in a common carotid artery and external jugular vein. A temporary feeding tube was briefly inserted through the endotracheal tube to instill Surfactant (Curosurf, Chiesi Farmaceutici Spa, Parma, Italy) into the airways. The umbilical cord was milked and subsequently clamped and cut. The PT lambs were kept prone in a veterinary sling mounted on a heated NICU bed. Tenders managed the PT lambs 24/7.

Sedation was accomplished by pentobarbital sodium (2-4 mg/Kg, iv) as needed; Abbott Laboratories, North Chicago, TL,) and buprenorphine hydrochloride (5 mcg/Kg; Reckitt & Colman Pharmaceuticals, Richmond, VA). Monitoring included body movement, heart rate, blood pressure, rectal temperature, and urine output. Resuscitation was standardized for all lambs, using a customized resuscitation device connected to a Drager VN-500 infant ventilator (Lubeck, Germany) (2). The PT lambs were resuscitated with two sustained lung inflations (24 cmH₂O for 35 sec each, with a 5-sec expiratory pause before and after each sustained lung inflation). The two sustained lung inflations were separated by 55 sec of intermittent positive airway pressure, after which intermittent positive airway pressure was continued (respiratory rate (RR)=60 bpm, inspiratory time (Ti)=0.3 sec, peak inspiratory pressure (PIP)=24 cmH₂O, positive end-expiratory pressure (PEEP)=8 cmH₂O). The inventors used a lung recruitment method that used 0.5 cmH₂O increments in PEEP every 30 sec, until 10 cmH₂O PEEP was reached, after which PEEP was stepwise decreased by 0.5 cmH₂O decrements every 30 sec, until reaching 8 cmH₂O. The lambs were weighed and placed on a heated NICU bed. The ventilator provided synchronized intermittent mandatory ventilation that was pressure controlled, with warmed and humidified gases. Initial ventilator settings were respiratory rate of 60 breaths/min, inspiratory time of 0.32 sec, peak inspiratory pressure (PIP) of 21 cmH₂O, and positive end-expiratory pressure (PEEP) of 8 cmH₂O. The target expiratory tidal volume, measured by the ventilator, was 5 to 7 mL/Kg.

Arterial blood gases were obtained starting at 15 min of postnatal life and taken every 15 min for the first 90 min of postnatal life. FᵢO₂ was decreased to attain a target oxygenation of O₂ saturation 88-94% by pulse oximetry (Model SurgiVet V9200IBP/Temp, Smith Medical ASD, Inc., St. Paul, MN). PIP was adjusted to attain a target PaCO₂ between 45 and 60 mmHg. The resultant pH range was between 7.25-7.35.

The PT lambs received parenteral (iv) dextrose infusion to maintain plasma glucose between 60 and 90 mg/dL. The lambs were prophylactically treated with penicillin G (8×10⁵ units, iv; WG Critical Care LLC, Paramus, NJ) and amikacin (500 mg; Avet Pharmaceutical, Inc, East Brunswick, NJ). Arterial blood gases (PaO₂ and PaCO₂) and pH were measured at 15, 60, and 120 min of postnatal life. Within 30 min of delivery, the lambs were treated with a loading dose of caffeine citrate (15 mg/Kg, given IV over 90 min, Sagent Pharmaceuticals, Schaumburg, IL), followed by maintenance treatment (5 mg/Kg, given IV every 24 h for 7d).

Orogastric feeding of ewe's colostrum was started at ˜3 h of postnatal life (3 mL). The volume of colostrum was gradually increased as tolerated, with a target of ˜60 kcal/Kg/d over the first week of postnatal life.

The transition from PT lambs to former PT lambs began at the day of life (DOL) 7-8. Pentobarbital administration was stopped. Subsequently, the ventilator circuit was separated by ˜1 cm from the connecting piece to the endotracheal tube. FiO₂ was increased to 100%. Spontaneous breathing and tissue oxygen saturation were monitored. Once both were sustained, an uncuffed nasal tube (Murphy tube, 3.0-3.5 mm ID) was inserted into one nasal passage. The tube's tip reached the mid-length of the nasal cavity (5-6 cm; the nasal cavity in fetal lambs at ˜128d gestation is ˜10 cm long) (6). The nasal tube was connected to the ventilator circuit and the ventilator was placed in the high-frequency oscillation (HFO) mode, with initial amplitude 20-25 cm H₂O, mean airway pressure 12 cm H₂O, HFO frequency 8 Hz, and I:E ratio 1:1. Conventional, positive-pressure background breaths were provided to support acceptable long-term ventilation, with initial settings of rate of 10 breaths per min, inspiratory time of 1.0 sec, and peak inspiratory pressure of the sigh breath of 25 cmH₂O.

Oxygenation was targeted for 88-94% saturation (PaO₂ 60 to 80 mmHg) by adjusting FiO₂. Ventilation was targeted for PaCO₂ between 45 to 60 mmHg by adjusting HFO amplitude and peak inspiratory pressure of the sigh breath. Lidocaine (1% solution; Hospira, Inc., Lake Forest, IL) was applied along the nostril to minimize pain and discomfort from the uncuffed nasal tube. The lambs were maintained on noninvasive respiratory support for ˜3d because of episodes of ineffective spontaneous respirations or episodes of apnea. When necessary, the lambs were stimulated to overcome ineffective spontaneous respiration. To overcome apneas, the lambs were stimulated and, if required, re-intubated. When re-intubated, the weaning process was repeated.

Orogastric feeding of ewe's colostrum (Kid & Lamb Colostrum Replacement, Land O Lakes, Arden Hills, MN) was started at ˜3 h of postnatal life (3 mL). The volume was gradually increased as tolerated, with a target over the first week of postnatal life of ˜60-90 kcal/kg/d. Parenteral dextrose was infused to maintain plasma glucose between 60 and 90 mg/dL.

Arterial blood and free-fall urine samples were collected every 24 h and analyzed for indicators of liver and kidney function (Associated Regional and University Pathologists (ARUP) Laboratories, Salt Lake City).

FPT and term reference lambs were vaccinated for Clostridium perfringens types C & D and tetanus by the veterinary staff. Vaccination removed gastrointestinal problems (diarrhea). The vaccination schedule was initial dose at 1 to 2 weeks' postnatal age for FPT lambs and at about 24 hours' postnatal age for term control lambs, followed by booster dose 2-3 weeks later.

At DOL9-10, the PT lambs were removed from all respiratory support and moved from their heated NICU bed to a heated floor pen to let them move freely (3, 4). During the initial days as FPT lambs, supplemental O₂ (blow-by via a cone) was necessary to maintain O₂ saturation at 88-94% by pulse oximetry, particularly when they slept. Need for supplemental O₂ support was typically 2 to 4d.

Matched term lambs were raised with the FPT lambs to provide a normal postnatal developmental reference. The term reference lambs were born spontaneously at term (˜150d) gestation and stayed with their ewe for ˜24 h to take colostrum. After ˜24 h, term control lambs were separated from their ewe for the remainder of the study to live with the FPT lambs.

Nutrition for the FPT and term reference lambs was provided by bottle, with an introduction to a nipple by filling the nipple with milk to let the lambs learn to suckle. Once suckling was effective, feedings were done by bottle. Ewe's milk feedings for the second week of postnatal life were 400 mL/d (140 mL/kg/d). Subsequent weekly milk feedings were 800 mL/d (220 mL/kg/d) from DOL14-20, 1,200 mL/d (240 mL/kg/d) from DOL21-30, and 1,800 mL/d (250 mL/kg/d) from DOL31-40. Milk feedings from DOL41 to 2 months corrected postnatal age were 2,700 mL/d (210 mL/kg/d), with no more than 450 mL/feeding to avoid feeding intolerance. Solid food (alfalfa pellets and hay) was introduced at ˜30d corrected postnatal age. Solid food and water were accessible ad libitum. All FPT and term reference lambs were weighed daily. The lambs were exercised daily.

Formulation and dosing of AVR-48. AVR-48 (3 mg/mL) was formulated in 0.9% sterile saline and filtered through a 0.2-micron syringe filter into 10 mL serum vials ready for dosing. The efficacy dose was determined in a pilot study by stepwise escalating the AVR-48 dose concentrations. The dose selection was based on the inventors' prior report on the mouse pup BPD model, where 10 mg/kg was the most effective dose.⁹ For the pilot study, the inventors tested 0 (n=2), 0.1 (n=1), 0.3 (n=1), 1.0 (n=2), 3.0 (n=3), and 9.0 (n=1) mg/kg/dose of AVR-48. The vehicle control groups received 0.9% sterile saline every 12 hours (h).

Study design. A randomized, blinded, placebo-controlled study design was used for both endpoints. For each endpoint study, one group of PT lambs was treated with vehicle (saline, intravenously, iv, over about 10 min) for 7d and thus served as control. Another group of PT lambs was treated iv with AVR-48 for 7d. Either treatment was started at 6 h of postnatal life to allow the PT lambs to stabilize after Cesarean-section delivery and resuscitation physiologically and to permit initiation of ventilator-induced lung injury. The inventors relied on visual identification of biological sex at the time of delivery in the absence of an ultrasound unit in the lamb intensive care unit. The inventors tracked sex as a biological variable, but the study could not assess differences in outcomes based on sex because of small sample sizes.

Respiratory system mechanics assessments with methacholine challenge. The respiratory system mechanics were assessed for the early-stage study (DOL1-10) and late-stage study (DOL10-90). For the early-stage research, the Drager ventilator recorded respiratory system mechanics every 5 min while the PT lambs were intubated and mechanically ventilated. The parameters were dynamic lung compliance and resistance. The inventors averaged the parameters every 12 h.

For the early and late-stage studies, after the lambs were extubated and transitioned to noninvasive respiratory support and spontaneously breathing (DOL7-10) and thereafter to DOL90, the inventors used forced oscillometry (FOT; model Tremoflo N-100; Thorasys, Thoracic Medical Systems Inc., Montreal, QC, Canada), with a filter (Humid-Vent Filter Small A, Teleflex Medical, Ireland). This instrument, validated in term and PT human infants,¹⁸ allows the measurement of respiratory system mechanics in uncooperative subjects (not intubated and breathing spontaneously) by applying a pressure stimulus at the airway opening and measuring the resulting flow. The inventors used a small canine mask (product number 93815026; Midmark Corp., Versailles, OH). The FOT device was calibrated daily, and the use followed the manufacturer's instructions. The inventors used the standard TremoFlo-supplied software without modifications. The software enables post hoc inspection of the measurement time course. As recommended by the manufacturer, any measurement with a leak was discarded. FOT measurements were made for the FPT lambs at 1 month corrected postnatal age (1 moC; DOL60) and 2 moC (DOL90). The comparison time points for term reference lambs were matched for corrected postnatal age.

FOT analysis was done on lambs that stood prone, supported by a veterinary sling. The lambs were given Lorazepam (0.1 mg/Kg, intramuscularly) to keep them calm. The mask was placed over their muzzle and held in place by a tender. The lambs were habituated to the sling and mask before testing was done. FOT was used to measure respiratory system resistance and reactance in four steps: (1) baseline, (2) bronchodilator (albuterol), (3) bronchoconstrictor (methacholine challenge), and (4) bronchodilator (albuterol). The inventors used an Aeroneb Pro nebulizer (Aerogen Ltd, Galway, Ireland) connected to the mask. The inventors repeatedly measured tissue O₂ saturation by pulse oximetry, heart rate, and blood pressure. After the baseline measurement, the inventors nebulized albuterol (1.5 mL; Hi-Tech Pharmacal Co., Amityville, NY) to dilate the airways in each lamb before methacholine challenge. Airway responsiveness was assessed during stepwise increases in nebulized methacholine (Provocholine; Pancap, Inc., Markham, Ontario, Canada), according to the ATS 1999 guideline¹⁹ and reported by the inventors for FPT and term reference lambs.¹⁴ Methacholine concentration was doubled for each step, starting from 0.06 mg/mL up to a maximum concentration of 4.00 mg/mL. At the end of the methacholine challenge steps, 1.5 mL of albuterol was nebulized to dilate the airways. All lambs tolerated the procedure, which took 45-60 min.

Neurobehavioral assessments. The inventors adapted tests for curiosity, using novel objects of different colors and shapes; socialization, using a nonreflective (dull) surface versus a reflective surface (mirror); and learning and memory, using a maze with a reward.²⁰ These tests were adapted from other studies that assessed the neurobehavior of sheep.²⁰⁻²⁵ Assessments were made over 2 weeks.

Habituation (open-field for exploration; days 1 and 2) was used to familiarize the lambs with the test, rest rooms, and tender. Habituation was accomplished on days 1 and 2 by a 10-minute stay in the test room, a 30-minute rest period in the rest room, and another 10-minute stay in the test room. Habituation was repeated the next day.

Tests were (1) curiosity, using novel objects; (2) socialization, using a non-reflective surface and a reflective surface; and (3) learning and memory (using a maze with a milk bottle reward). The tests were done on sequential days over two weeks. Tests were trials of 10-min each, with a 30-min rest period between trials. Each 10-min trial was video recorded, using the Noldus System (model EthoVision XT 17 with the Physiology Integration Module; Noldus Information Technology Inc., Leesburg, VA). The test room had tape on the floor that divided the floor into 9 zones for identification of movement direction and speed, inactivity, etc., in video playback. A ceiling camera recorded each test session. The camera was connected to a dedicated computer with the Noldus software to document lamb activity and time. The novel objects test required four trials on day 3. Trials 1 and 2 used two objects (brown and blue bowls) affixed to the floor. For trial 3, before the lamb was returned to the test room, the brown bowl was replaced with a novelx object (pink pitcher). For trial 4, before the lamb was returned to the test room, a new object (yellow ball) was added with the blue bowl and pink pitcher (3 objects). Analysis was the number of times and the time spent investigating each object.

The socialization test required two trials each on days 4 and 5. Two metallic surfaces were attached to a wall, one with a non-reflective and the other with a reflective surface. Analysis was the number of times and the time spent investigating each surface.

The following week, the learning and memory test required two trials on day 6. A maze made of solid metal panels was set up in the test room. A tender was in one corner of the maze, holding a bottle of milk, the reward for completing the maze. Once the tender was in place, the lamb entered the test room. Analysis was the time to reach the milk bottle reward.

Terminal tissue collection. The inventors followed the methods the developed for terminal tissue collection.^{14,17,26-28} Briefly, at the end of the early-stage study (DOL10) or late-stage study (DOL90 for FPT lambs; DOL60 for term reference lambs), the lambs were given ketamine (10-20 mg/Kg, intramuscularly) followed by face mask inhalation anesthesia with 1.0-2.5% isoflurane with O₂. Lambs were intubated and ventilated with a tidal volume of 5-7 mL/kg, and given heparin (1000 U, iv). The lambs were given Beuthanasia solution (0.25 mL/Kg, iv; Intervet Inc., Madison, NJ) followed by potassium chloride (10 mEq, iv; Hospira, Lake Forest, IL).

For morphological analyses, the entire left lung was insufflated with formalin and sampled by systematic, uniform, random methods for quantitative histology.²⁹ Morphometric and stereologic methods quantified radial alveolar count, secondary septal volume density, and thickness of distal airspace walls.²⁷ Alveolar capillary growth was quantified by a combination of immunohistochemistry to identify endothelial cells in lung tissue sections and quantitative histology.²⁷ For capillary surface density measurement, a recombinant anti-CD-34 antibody was used (ab81289, Abcam; Waltham, MA) for the capillary endothelial cell marker. Negative immunostain controls were omission of the primary antibody, omission of the secondary antibody, and substitution of the primary antibody with a species-matched, irrelevant antibody. Epithelium lining the air spaces was counterstained blue. The inventors used a computer-assisted true-color imaging system (Bioquant Life Science, Nashville, TN) to quantify the surface density of alveolar capillaries and the overlying epithelium.

For molecular analyses, the right caudal lobe was cut into 1 cm³ pieces (˜1 g each), again using systematic, uniform, random sampling methods. The lung pieces were from peripheral tissue devoid of visceral pleura and central airways, vessels, and connective tissue. The pieces were snap-frozen in liquid nitrogen and stored at −80° C. Homogenates of frozen tissue pieces were used for quantitative real-time RT-PCR and immunoblot analyses. Expression of mRNA was normalized to GAPDH. Primary antibodies for immunoblot analyses are summarized in Table 1. For immunoblot analysis, cleaved caspase 3 was used (EnzoADI-AAP-113, Enzo Life Sciences, Inc., Farmingdale, NY), PCNA (ab2426, Abcam, Cambridge, MA), and VEGF-R₂ (sc-393163, Santa Cruz Biotechnology, Dallas, TX) proteins. Positive controls for immunoblot were the respective native proteins. Protein abundance was normalized, using Memcode (P124585, Fisher Scientific, Pittsburgh, PA).

TABLE 1
List of antibodies used in immunohistochemistry and Western blot

| | | | | City, State | | |
|---|---|---|---|---|---|---|
| Order | Antibody name | Company | Catalog # | and Country | Molecular weight | |
| 1 | Casp3 | ENZO | ENZO# ADI- | Farmingdale, | ~36 | kDa; |
| | | | AAP-113-F | NY, USA | | |
| 2 | FLK1 | Abacm | ab134191 | Waltham, | immature ~150 kDa; | |
| | | | | MA, USA | intermediate | |
| | | | | | glycosylated ~200 | |
| | | | | | kDa; mature | |
| | | | | | glycosylated ~230 kDa; | |
| 3 | VEGF | Santa Cruz | 147 | Dallas, TX, | monomer 21 kDa; dimer | |
| | | Biotech | | USA | 42 kDa | |
| 4 | PCNA | Santa Cruz | sc-56 | Dallas, TX, | ~36 | kDa |
| | | Biotech | | USA | | |
| 5 | SP-B | Santa Cruz | sc-133143 | Dallas, TX, | ~9 | kDa |
| | | Biotech | | USA | | |
| 6 | TLR4 | ThermoFisher | PA5-23284 | Waltham, | 97-116 | kDa |
| | | Scientific | | MA, USA | | |
| 7 | Icam1 | ThermoFisher | 10831-1-AP | Waltham, | 85-110 | kDa |
| | | Scientific | | MA, USA | | |
| 8 | TNFα | ThermoFisher | PBOTNFI | Waltham, | 26 | kDa |
| | | Scientific | | MA, USA | | |
| 9 | SP-A | ProteinTech | 11850-1-AP | Rosemont, | 26 | kDa |
| | | Group, Inc | | IL, USA | | |
| 10 | IL-6 | LS Bio an | LS-C292466- | Newark, CA, | 24 | kDa |
| | | Absolute Biotech | 100 | USA | | |
| 11 | IL-10 | ThermoFisher | PA5-79457 | Waltham, | 18 | kDa |
| | | Scientific | | MA, USA | | |
| 12 | IL-1β (cleaved) | Cell Signaling | 83186 (D3A3Z) | Danvers, | 17 | kDa |
| | | Technology, Inc | | MA, USA | | |
| 13 | IL1β (precusor) | ProteinTech | 16806-1-AP | Rosemont, | 30-35 | kDa |
| | | Group, Inc | | IL, USA | | |
| 14 | iNOS | ProteinTech | 22226-1-AP | Rosemont, | 110-130 | kDa |
| | | Group, Inc | | IL, USA | | |

### Demographics

TABLE 2
Demographics of PT lambs for the early-stage study (10 d)

| | | Birth wt | | Gestation at | End wt | Survival |
|---|---|---|---|---|---|---|
| Lambs | Treatment | (kg) | Sex | delivery (d) | (kg) | (d) |
| 1 | Saline | 1.90 | M | 129 | 2.00 | 10 |
| 2 | Saline | 2.25 | M | 128 | 2.10 | 10 |
| 3 | Saline | 3.40 | F | 128 | 3.25 | 10 |
| 4 | Saline | 2.70 | M | 128 | 2.50 | 14^{a} |
| 5 | Saline | 2.30 | F | 128 | 3.25 | 15^{b} |
| 6 | Saline | 3.40 | M | 128 | 3.25 | 10^{c} |
| 7 | Saline | 2.75 | F | 126 | 2.2 | 11^{d} |
| 8 | 0.1 mg/kg | 1.2 | F | 128 | 1.20 | 10 |
| 9 | 0.3 mg/kg | 1.4 | M | 128 | 1.20 | 10 |
| 10 | 1.0 mg/kg | 2.85 | M | 129 | 1.50 | 10 |
| 11 | 1.0 mg/kg | 2.25 | M | 129 | 1.50 | 10 |
| 12 | 3.0 mg/kg | 1.90 | F | 129 | 1.90 | 10 |
| 13 | 3.0 mg/kg | 2.24 | F | 129 | 1.70 | 10 |
| 14 | 3.0 mg/kg | 3.10 | M | 128 | 2.15 | 10 |
| 15 | 3.0 mg/kg | 2.45 | F | 128 | 2.50 | 11^{e} |

TABLE 3
Demographics of FPT lambs for the late-
stage study (2 moC PNA or 90 d)

| | | Birth wt | | Gestation at | End wt | Survival |
|---|---|---|---|---|---|---|
| Lambs | Treatment | (kg) | Sex | delivery (d) | (kg) | (d) |
| 1 | Saline | 3.15 | F | 128 | 21.8 | 92 |
| 2 | Saline | 2.30 | M | 128 | 20.3 | 92 |
| 3 | Saline | 3.10 | F | 126 | 24.8 | 92 |
| 4 | 3.0 mg/kg | 2.90 | F | 129 | 24.4 | 92 |
| 5 | 3.0 mg/kg | 3.90 | M | 129 | 27.0 | 92 |
| 6 | 3.0 mg/kg | 3.05 | F | 128 | 27.5 | 92 |
| 7 | 3.0 mg/kg | 3.25 | M | 128 | 24.3 | 92 |

Four other FPT vehicle lambs required early euthanasia for humane reasons for expected complications. The complications occurred at (a) DOL14 for acute kidney injury, (b) DOL15 for lymphangiectasis (per necropsy by the State pathologist at Utah State University), (c) DOL10 for inadequate left ventricular function and aspirated milk, and (d) DOL11 for severe respiratory distress and failure to wean, with ¾ of lung mass that was consolidated/atelectatic. e) One other FPT AVR-48 lamb required early euthanasia for humane reasons for expected complications. The complication occurred on DOL11, a massive amount of swallowed air while the lamb was on noninvasive respiratory support (the air could not be evacuated from the stomach chambers); this expected complication was unrelated to AVR-48 treatment.

ELISA and BCA assay methods. BCA assay: The lamb BAL fluid samples (10d and 90d terminal studies) were centrifuged to remove the cell pellets, and the supernatant was used to assess the total protein using the Pierce™ BCA Protein Assay Kits, which were read using a BioTek synergy H1 microplate reader (Fisher Scientific, Toronto, ON, Canada).

ELISA: The serum and bronchoalveolar lavage (BAL) fluid samples were used to quantify cytokine and soluble CD163 (sCD163) concentrations. The ELISA experiment analysis used commercially available ELISA kits, which were quantified using a plate reader. The inventors used GraphPad Prism V. 10.4. All sheep (ovine) ELISA kits were used within the expiry dates. Ovine IL-1β(Cat #ELO-IL1β), and ovine IL-6 (Cat #ELO-IL6) ELISA kits were purchased from RayBiotech, Peachtree Corners, GA, USA, sheep IL-10 (Cat #E12817Sh-96) was purchased from Lifeome Biolabs, Oceanside, CA, sheep CD163 (cat #MBS9364870) was purchased from MyBioSource, Inc. San Diego, CA, and Pierce™ BCA Protein Assay Kit (Cat #23227) from Thermo Fischer Scientific, Rockford, IL.

Pharmacokinetic study. The pharmacokinetics (PK) of AVR-48 were determined using plasma from the PT lambs following twice daily IV infusion (10 min) of AVR-48 every 12 h for 7 days. Tested doses were 0 (n=2), 0.1 (n=1), 0.3 (n=1), 1.0 (n=2), 3.0 (n=3), and 9 (n=1) mg/kg. PK evaluation for AVR-48 was only performed for DOL1 (12 time points between 0 and 12 h after the initial dose; volume of plasma samples through DOL2-7 was inadequate for PK evaluation). Quality-controlled plasma bioanalytical data were transferred from the Bioanalytical group to the PK Scientist of the Biopharmaceutics Department of Pharmascience Inc. Samples were analyzed, using Phoenix WinNonlin software, version 8.1 (Certara, USA), using a previously standardized HPLC/LC/MS method.

Statistical assessments. Results are presented as mean±SD. The inventors used GraphPad for statistical assessments. The physiological results were analyzed by ANOVA and a mixed model because of some missing data values on DOL6 or 7. Quantitative histological results were analyzed by unpaired t-test. Molecular analyses were performed by the Mann-Whitney U test. The inventors accepted p≤0.05 for identifying statistical differences.

Pharmacokinetics of AVR-48 after intravenous (iv) dosing in preterm lambs. For dose comparison, AVR-48 (0.1, 0.3, 1.0, 3.0, or 9 mg/kg/dose, iv) was given every 12 h for 7d and change in concentration was plotted at different time points. Pharmacokinetics (PK) evaluation for the efficacy dose of AVR-48 (3.0 mg/kg) was done from 0 to 12 h after the initial dose (FIG. 1A). Maximum plasma concentration (Cₘₐₓ) occurred at 0.25 h for all tested doses (FIG. 1A). The theoretical Cₘₐₓ (concentration extrapolated to 0 hr; C₀) for AVR-48 (3.0 mg/kg) was followed by a bi-exponential decline of the drug levels indicated by its half-life (T_{1/2}), and was estimated to be 0.64+0.24 hr (Table 4). Exposure to AVR-48 (AUC₍₀₋ₜ₎) increased dose-relatedly over the dose range tested. The volume of distribution (V_{d}) for AVR-48 was estimated to be 668.4±129.0 mL/kg, and clearance (CL) was estimated to be 668.4±129.0 mL/hr/kg. AVR-48 (3.0 mg/kg/dose; efficacious dose) had a Cₘₐₓ of 10.73 μM (3885.7±1458.7 ng/mL) followed by a linear decline in drug concentration (FIG. 1A) in lamb plasma. For reference, the Cₘₐₓ of AVR-48 in mouse pups' plasma at the efficacy dose of 10 mg/kg intraperitoneal injection was 5.7 μM. The repeat dose Cₘₐₓ was consistent and provided the expected area under the curve (AUC) concentration level of 3886.6±619.7 ng/mL after 15 minutes of each dosing (FIG. 1), correlating to the AUC_{0-12 h} was estimated to be 4222.9±1835.9 hr*ng/mL (Table 4).

TABLE 4
Combined PK Parameters in male and Female lamb plasma following iv bolus
administration of AVR-48 on Day 1 (time point 0 to 12 hr post dose)

| | | Cₘₐₓ | C₀ | AUC₍₀₋ₜ₎ | Tₗₐₛₜ | T_{1/2} | V_{d} | CL |
|---|---|---|---|---|---|---|---|---|
| Dose (bid) | Tₘₐₓ | (ng/mL ± | (ng/mL ± | (hr*ng/mL ± | (hr ± | (hr ± | (mL/kg ± | (mL/hr/kg ± |
| (mg/kg) | (hr) | SD) | SD) | SD) | SD) | SD) | SD) | SD) |
| 3.00 | 0.25 | 3885.7 ± | 5138.8 ± | 4222.9 ± | 4.75 ± | 0.64 ± | 668.4 ± | 791.6 ± |
| | | 1458.7 | 2954.2 | 1835.9 | 2.95 | 0.24 | 129.0 | 277.7 |

AVR-48 dose-response effects for lung outcomes. Early-stage (10d) and late-stage (90d) studies were conducted. For both studies, AVR-48 treatment was for 7d during IMV. Subsequently, noninvasive respiratory support was provided for 3d. During the 10d period, BPD-like symptoms were diminished in a dose-related manner by AVR-48. Significant improvements occurred in respiratory severity score (RSS) and lung structure at the 3.0 mg/kg dose. RSS was improved moderately at 1.0 mg/kg AVR-48 and significantly with the 3.0 mg/kg/dose. Moreover, quantitative histology showed that radial alveolar count (RAC) was statistically higher in the AVR-48-treated PT lambs than in the vehicle lambs). Conversely, distal airspace walls were statistically thinner in the AVR-48-treated PT lambs compared to the vehicle lambs. The cumulative assessments determined 3.0 mg/kg as the efficacious dose for AVR-48.

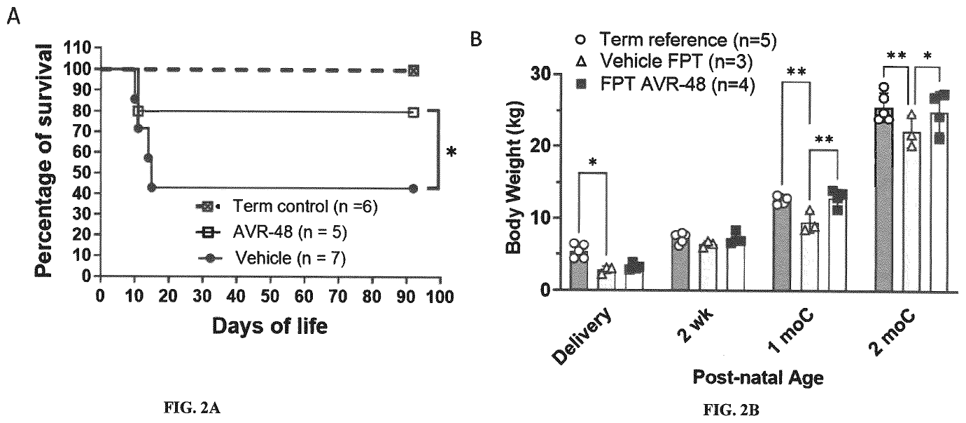

AVR-48 improved survival, growth, and lung outcomes early and late. For the early-stage study, almost 100% of PT lambs survived in both groups (FIG. 2A). For the late-stage study, survival significantly declined from d10-d90 for the FPT vehicle lambs (40%, 4/7) compared to the FPT AVR-48 lambs (80%, 1/5) (FIG. 2A). Body weight significantly increased in FPT AVR-48 lambs compared to FPT vehicle lambs (FIG. 2B). The growth of the FPT AVR-48 lambs was similar to that of the term reference lambs (FIG. 2B).

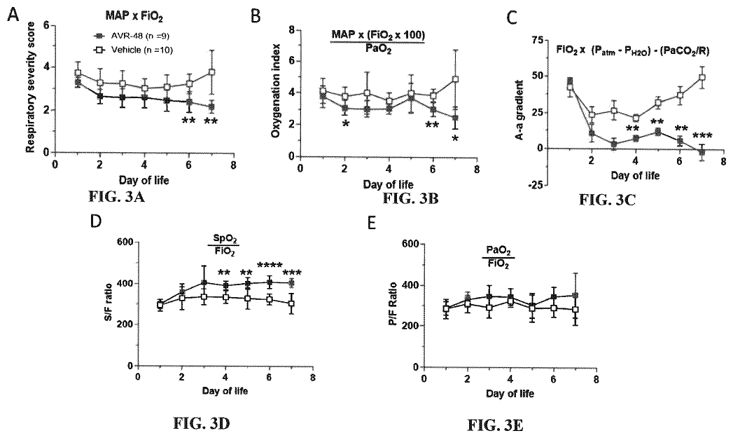

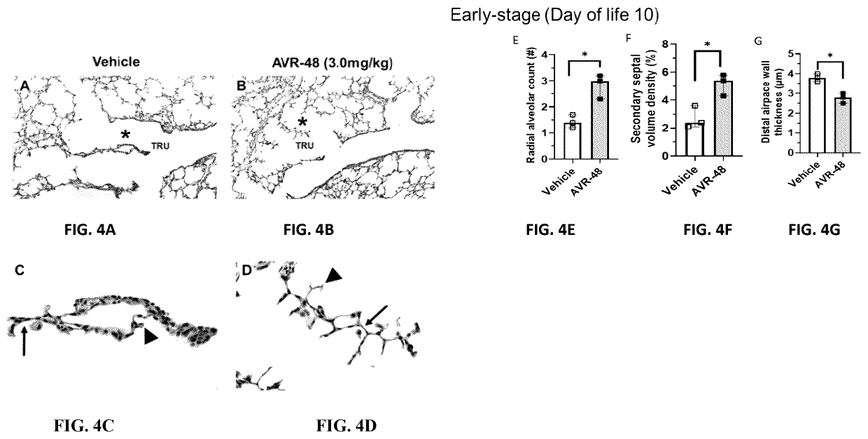

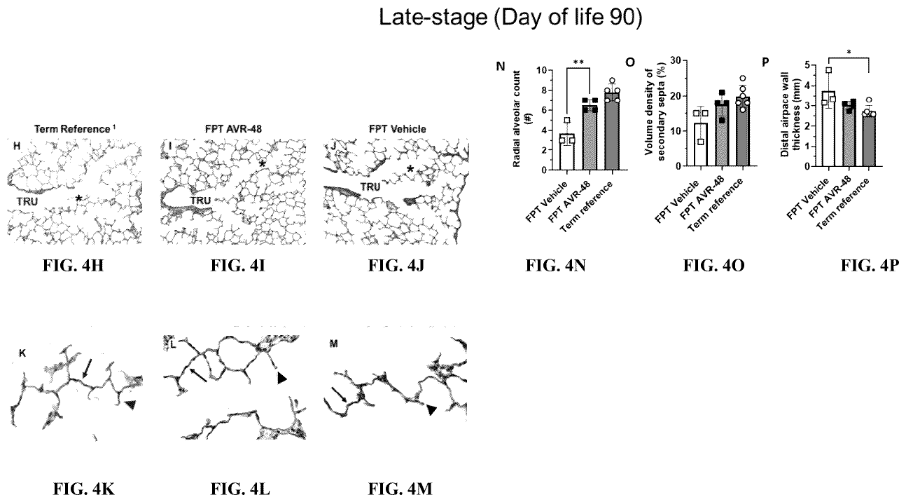

For the early-stage study, respiratory system and cardiovascular physiology parameters, such as RSS, oxygenation index (01), SpO₂/FiO₂ (SF) ratio,³⁰ and Alveolar-arterial (A-a) gradient were significantly improved in the AVR-48 PT lambs compared to the vehicle PT lambs (FIGS. 3A-D). AVR-48-treated lambs had significantly better development of terminal respiratory units (TRUs) (FIGS. 4A-D, 4H-M) for both the early-stage and late-stage studies. TRUs include the terminal bronchioles and subsequent alveoli and are primarily responsible for respiratory gas exchange. The morphometrical and stereological analyses of the lung tissue showed a significant increase in radial alveolar count (RAC) in the AVR-48 groups of lambs compared to the vehicle groups of lambs at 10d (FIG. 4E) and 90d (FIG. 4N). Secondary septal volume density was significantly higher, and distal airspace wall thickness was significantly lower in the AVR-48 PT lambs compared to the vehicle PT lambs at 10d (FIG. 4F). At 90d, secondary septal volume density was higher in the AVR-48 lambs (but not statistically different) (FIG. 4O). Distal airspace wall thickness (FIG. 4G) was significantly thinner in the AVR-48 PT lambs compared to the vehicle PT lambs at 10d (FIG. 4O). At 90d, distal airspace wall thickness was thinner (but not statistically different) in the FPT AVR-48 lambs relative to the FPT vehicle lambs (FIG. 4P). Each parameter in the FPT AVR-48 lambs was similar to the term reference lambs (FIGS. 4N-P).

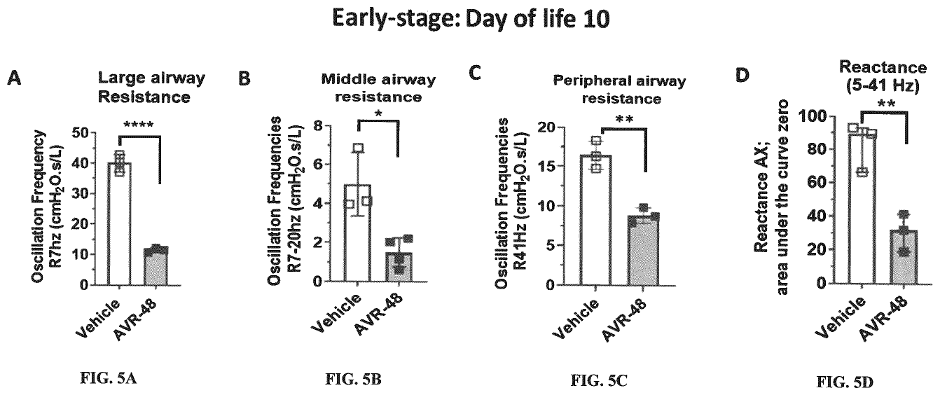

AVR-48 improved respiratory system resistance and reactance at early and late stages. Respiratory system mechanics (resistance and reactance) were measured by the FOT, using Tremoflo N-100. For the early-stage study, AVR-48-treated PT lambs (n=3) had significantly lower resistance in large (5 Hz), middle (7-20 Hz), and small (41 Hz) airways compared to vehicle PT lambs (FIGS. 5A-D). Airway reactance AX (area under the curve below zero) was significantly lower in the AVR-48 PT lambs than in vehicle PT lambs.

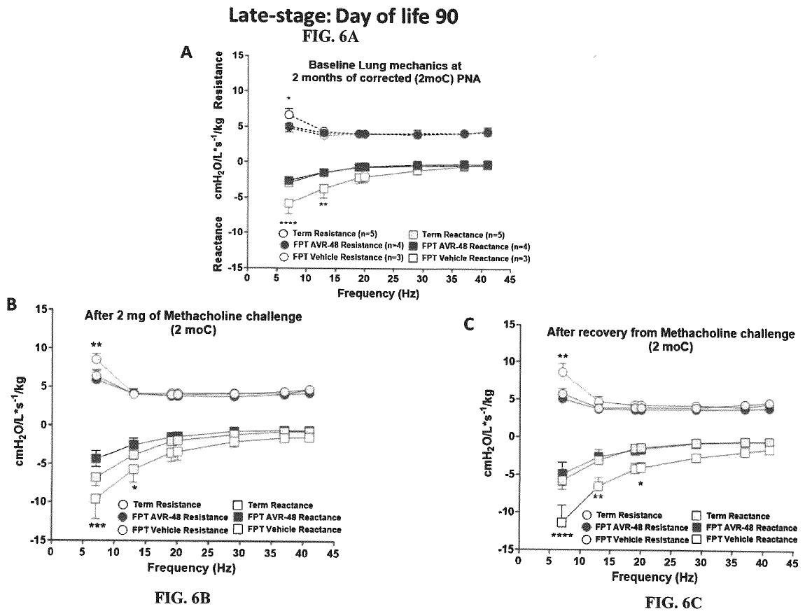

For the late-stage study, FOT measurements were made in FPT lambs and term reference lambs on d60 and d90. The comparison time points for term reference lambs were matched for corrected postnatal age. Results are reported for the 90d assessment (FIGS. 6A-C) and for 60d. Measurements were made at baseline and after methacholine challenge to induce airway smooth muscle contraction, following the ATS 1999 protocol.³¹ Albuterol was nebulized afterward to dilate the airways. Baseline resistance at 5 Hz (upper and large airways) was significantly lower for the FPT AVR-48 lambs compared to the FPT vehicle lambs. Reactance at 5 and 10 Hz (upper and large airways) was significantly less negative for the FPT AVR-48 lambs compared to the FPT vehicle lambs (FIG. 6A). Baseline resistance and reactance were the same (superimposed in the graph) between the FPT AVR-48 lambs and the term reference lambs. No differences were detected at the higher frequencies. Methacholine challenge revealed that the FPT AVR-48 lambs had significantly lower resistance at 5 Hz (upper and large airways) compared to the FPT vehicle lambs (FIG. 6B). For the same FPT lamb groups, reactance at 5 and 10 Hz (upper and large airways) was significantly less negative for the FPT AVR-48 lambs compared to the FPT vehicle lambs. After albuterol nebulization, the FPT AVR-48 group had significantly lower resistance and less negative reactance at 7-20 Hz frequencies (middle and peripheral airways) compared to FPT vehicle lambs (FIG. 6C). The respiratory system mechanics for the FPT AVR-48 lambs had no statistical differences from the term reference lambs.

AVR-48 improved long-term neurobehavioral outcomes at late-stage study. Brain injury in survivors of preterm birth and the NICU setting is linked to poor neurodevelopmental outcomes later in life, with worse outcomes with a higher degree of chronic lung injury.³² Preterm lambs that have chronic lung disease develop non-cystic, non-hemorrhagic diffuse brain damage.³³

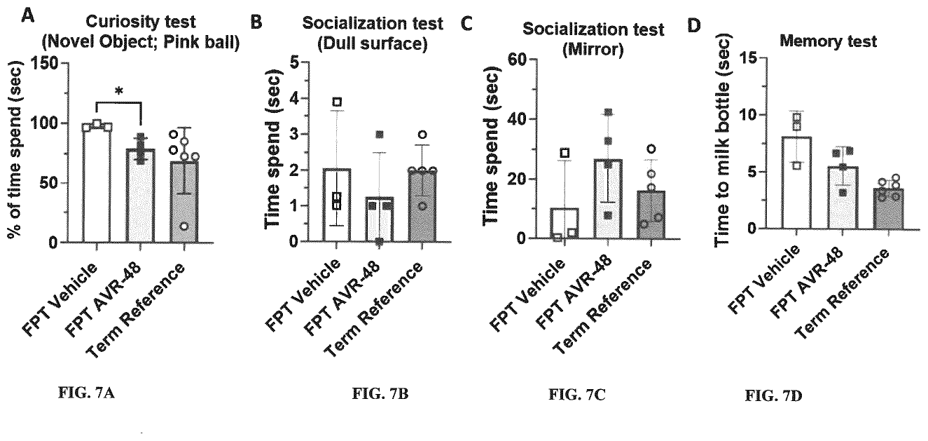

The inventors adapted tests as part of the late-stage study for curiosity behavior test, using novel objects of different colors and shapes; socialization, using a nonreflective (dull) surface versus a reflective surface (mirror); and learning and memory, using a maze with reward (FIGS. 7A to 7D).²⁰ FPT lambs were tested twice, once at 1 month corrected postnatal age (1 moC PNA or d60) and again at 2 months corrected postnatal age (2 moC PNA or d90; equivalent to 12-18 months of postnatal age in humans when both species wean from milk).³⁴ Results are reported for the 2 moC PNA (d90) assessment period.

The curiosity (novel objects) test showed that the FPT AVR-48 lambs spent significantly less time looking at the novel object, the pink ball, compared to the FPT vehicle lambs (FIG. 7A) and was comparable to the time spent by the term reference lambs (p=0.871). In other words, the FPT AVR-48 and term reference lambs were curious about all the test objects. The socialization test showed that the FPT AVR-48 lambs spent less time (not significant) looking at the dull surface and more time (not significant) at the mirror compared to the FPT vehicle lambs (FIGS. 7B, C). The learning and memory test showed that the FPT AVR-48 lambs navigated the maze to reach the milk bottle reward significantly faster than the FPT vehicle lambs (FIG. 7D).

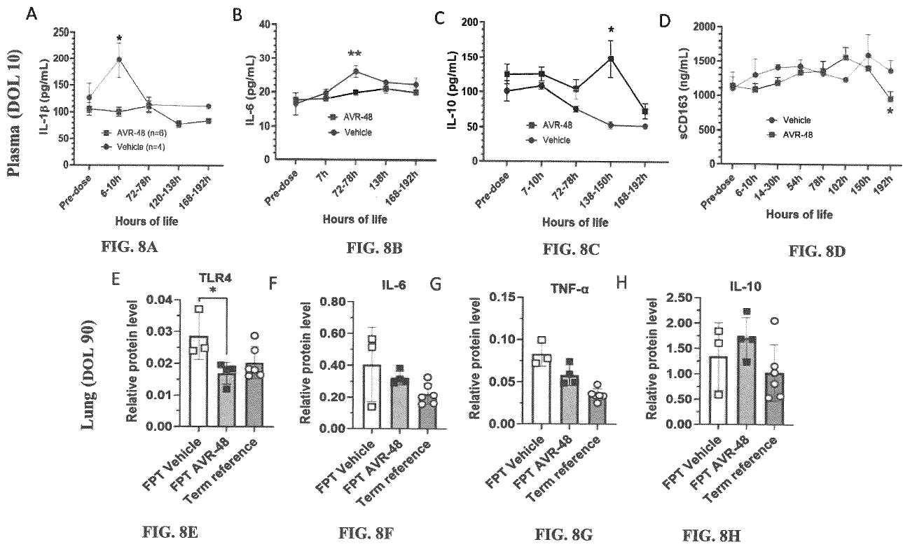

AVR-48 modulated systemic and pulmonary inflammatory markers early and late. For the early-stage study, plasma concentrations of pro-inflammatory cytokines IL-1β and IL-6 were significantly lower for the AVR-48 PT lambs compared to the vehicle PT lambs at 6-10 h and 72-75 h, respectively (FIGS. 8A and B). Conversely, the anti-inflammatory cytokine IL-10 was significantly higher in plasma from the AVR-48 PT lambs compared to the vehicle PT lambs at 138-150 h (FIG. 8C).

For the late-stage study, plasma soluble (s)CD163 significantly lower in the FPT AVR-48 lambs compared to the FPT vehicle lambs at 192 h (FIG. 8D).

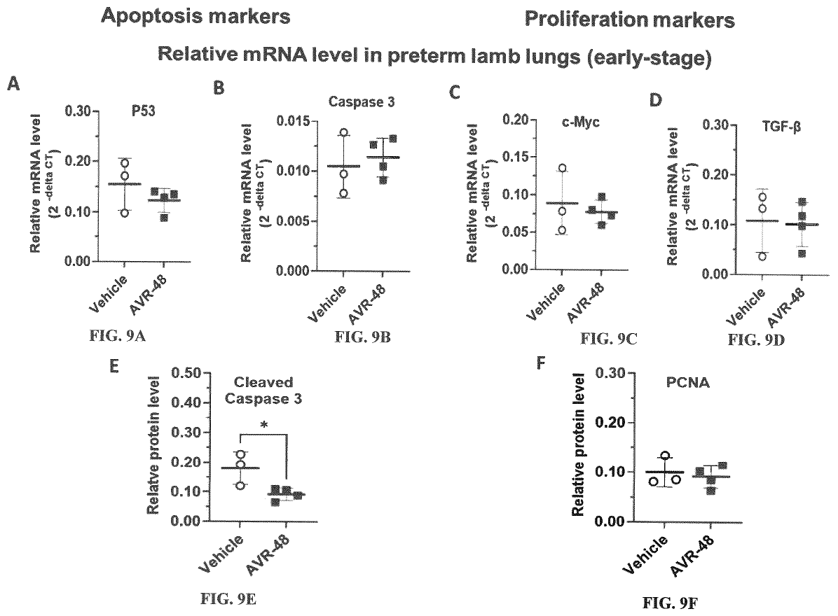

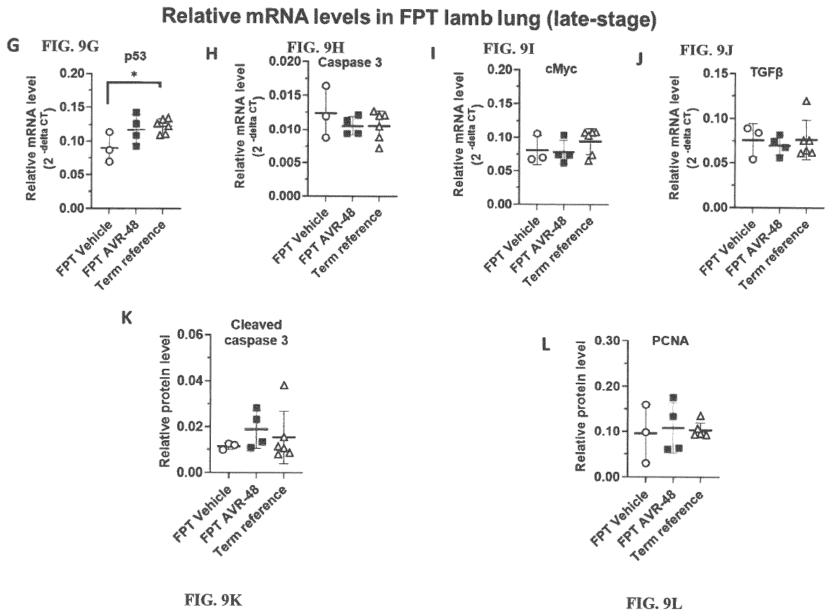

Peripheral lung tissue homogenates were analyzed for protein concentration of TLR4 and pathway-related cytokines. For the early-stage study, no differences were detected. For the late-stage study, TLR4 protein concentration was significantly lower in the FPT AVR-48 lambs compared to FPT vehicle lambs (0.028±0.010 vs 0.007±0.004) (FIG. 8E). The concentration of TLR4 protein in the lung tissue of the FPT AVR-48 lambs was comparable to that for the term reference lambs. IL-6 and TNF-α appeared lower (not significant) in the FPT AVR-48 lambs compared to the FPT vehicle lambs (FIGS. 8F and G). Conversely, IL-10 protein concentration appeared higher (not significant) in the FPT AVR-48 lambs compared to the FPT vehicle lambs (FIG. 8H). FIGS. 9A to 9L shows the results for the mRNA expression of TLR4 and pathway-related cytokines in the early and late studies.

Increased total protein in BALf represents increased vascular permeability and pulmonary edema status of the lung. The inventors observed that AVR-48 treatment decreased total protein concentration compared to vehicle-treated PT lambs at 10 days; however, no difference was observed in the BALf from FPT lambs at 90 days.

Apoptosis, proliferation, permeability markers in lung tissue homogenates after AVR-48 treatment at the end of the early and late-stage studies. Analysis of lung homogenates for changes in apoptosis and proliferation molecular markers related to PT lung development revealed that at DOL10, the lung tissues had a significantly lower level of cleaved caspase-3 (FIG. 9E) protein levels in AVR-48 treated PT lamb lungs compared to vehicle lamb lungs indicating decreased cellular apoptosis in lungs and supporting to the increased RAC results in lung morphometrical analysis (FIG. 4E). No significant difference was observed between vehicle and AVR-48 treated lamb lung samples when analyzed for the mRNA levels for p53, caspase-3, c-Myc, and TGF-β (FIGS. 9A-D). In the 90-day study samples, P53 (anti-apoptotic marker) is higher in both AVR-48 treated and term reference lamb lungs than FPT vehicle lamb lungs (FIG. 9G). In addition, there was no significant difference in the mRNA and protein levels of the other apoptosis and proliferation markers between AVR-48-treated FPT lamb lungs and term reference lamb lungs, demonstrating full recovery from respiratory distress and normal lung development after AVR-48 treatment (FIGS. 9H-L) supporting the previous results in FIG. 6 for improved lung mechanics and compliance at 90 DOL.

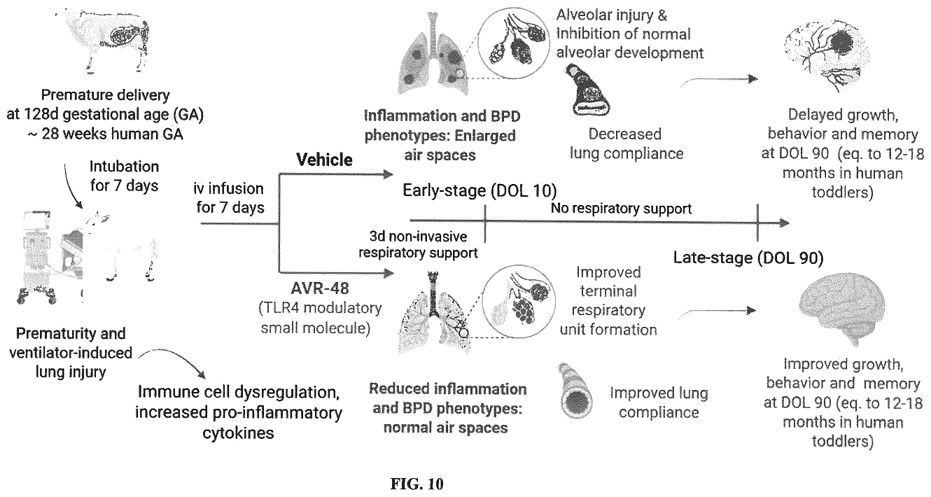

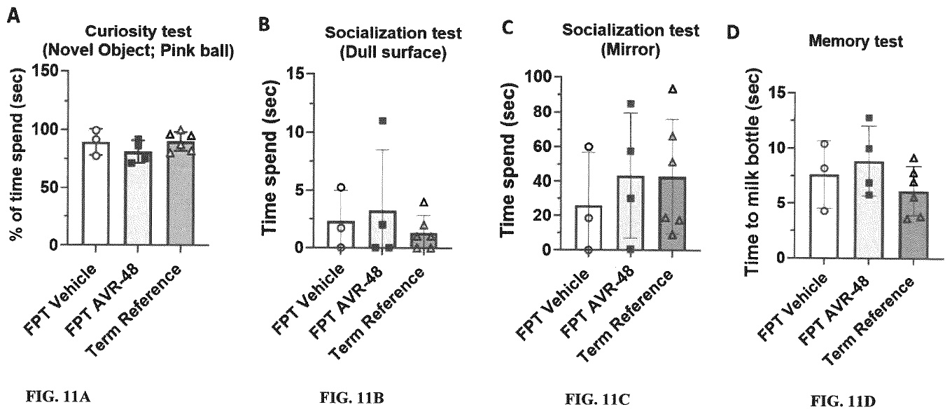

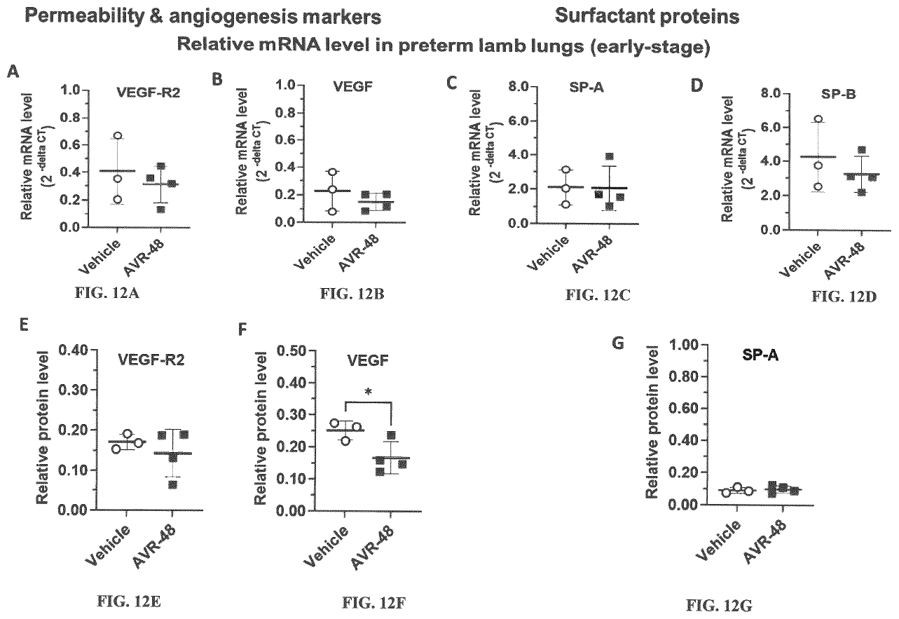

Summary of the effect of AVR-48 intravenous treatment on preterm lambs is depicted in the FIG. 10.

Clinical biochemistry. Plasma and urine samples were analyzed by Associated Regional and University Pathologists (ARUP) at the University of Utah. Plasma renal function parameters are urea nitrogen and creatinine. Plasma hepatic function parameters are alkaline phosphatase, aspartate aminotransferase, alanine aminotransferase, total protein, albumin, bilirubin total, and direct bilirubin. In summary, the clinical parameters in the AVR-48 treated lambs were within the normal range for lambs.

Physiological parameters for lambs in the early and late-stage studies. Physiology results were obtained including cardiovascular physiology, temperature, respiratory rates, indices for oxygenation, ventilation indices, plasma pH, bicarbonate, base excess, plasma electrolytes, plasma glucose, hematocrit (Hct), total serum protein (Tsp), lactate, enteral feeding, milk residual, and hematology for early and late-stage studies. Overall, AVR-48 treatment had no adverse effect on the above physiological outcomes.

PT lamb model and methods for all outcome measures. Surgical preparation and management of lamb groups. Delivery and clinical management methods for PT lambs (6-8) and FPT lambs (3, 4). Briefly, time-pregnant ewes that had single or twin fetuses at ˜128d (˜85%) gestation (term ˜150d gestation) were used. The fetuses were exposed to antenatal steroids (dexamethasone) at ˜48 h and ˜24 h before Cesarean-section (exit-procedure) delivery. An endotracheal tube was inserted, and catheters were placed in a common carotid artery and external jugular vein. A temporary feeding tube was briefly inserted through the endotracheal tube to instill Surfactant (Curosurf; Chiesi Farmaceutici Spa, Parma, Italy) into the airways. The umbilical cord was milked and subsequently clamped and cut. The PT lambs were kept prone in a veterinary sling mounted on a heated NICU bed. Tenders managed the PT lambs 24/7.

Sedation was accomplished by pentobarbital sodium (2-4 mg/Kg, iv) as needed; Abbott Laboratories, North Chicago, IL,) and buprenorphine hydrochloride (5 mcg/Kg; Reckitt & Colman Pharmaceuticals, Richmond, VA). Monitoring included body movement, heart rate, blood pressure, rectal temperature, and urine output. Resuscitation was standardized for all lambs, using a customized resuscitation device connected to a Drager VN-500 infant ventilator (Lubeck, Germany) (2). The PT lambs were resuscitated with two sustained lung inflations (24 cmH₂O for 35 sec each, with a 5-sec expiratory pause before and after each sustained lung inflation). The two sustained lung inflations were separated by 55 sec of intermittent positive airway pressure, after which intermittent positive airway pressure was continued (respiratory rate (RR)=60 bpm, inspiratory time (Ti)=0.3 sec, peak inspiratory pressure (PIP)=24 cmH₂O, positive end-expiratory pressure (PEEP)=8 cmH₂O). The inventors used a lung recruitment method that used 0.5 cmH₂O increments in PEEP every 30 sec, until 10 cmH₂O PEEP was reached, after which PEEP was stepwise decreased by 0.5 cmH₂O decrements every 30 sec, until reaching 8 cmH₂O. The lambs were weighed and placed on a heated NICU bed. The ventilator provided synchronized intermittent mandatory ventilation that was pressure controlled, with warmed and humidified gases. Initial ventilator settings were respiratory rate of 60 breaths/min, inspiratory time of 0.32 sec, peak inspiratory pressure (PIP) of 21 cmH₂O, and positive end-expiratory pressure (PEEP) of 8 cmH₂O. The target expiratory tidal volume, measured by the ventilator, was 5 to 7 mL/Kg.

Arterial blood gases were obtained starting at 15 min of postnatal life and taken every 15 min for the first 90 min of postnatal life. FᵢO₂ was decreased to attain a target oxygenation of O₂ saturation 88-94% by pulse oximetry (Model SurgiVet V9200IBP/Temp, Smith Medical ASD, Inc., St. Paul, MN). PIP was adjusted to attain a target PaCO₂ between 45 and 60 mmHg. The resultant pH range was between 7.25-7.35.

The PT lambs received parenteral (iv) dextrose infusion to maintain plasma glucose between 60 and 90 mg/dL. The lambs were prophylactically treated with penicillin G (8×10⁵ units, iv; WG Critical Care LLC, Paramus, NJ) and amikacin (500 mg; Avet Pharmaceutical, Inc, East Brunswick, NJ). Arterial blood gases (PaO₂ and PaCO₂) and pH were measured at 15, 60, and 120 min of postnatal life. Within 30 min of delivery, the lambs were treated with a loading dose of caffeine citrate (15 mg/Kg, given IV over 90 min, Sagent Pharmaceuticals, Schaumburg, IL), followed by maintenance treatment (5 mg/Kg, given IV every 24 h for 7d).

Orogastric feeding of ewe's colostrum was started at ˜3 h of postnatal life (3 mL). The volume of colostrum was gradually increased as tolerated, with a target of ˜60 kcal/Kg/d over the first week of postnatal life.

The transition from PT lambs to former PT lambs began at the day of life (DOL) 7-8. Pentobarbital administration was stopped. Subsequently, the ventilator circuit was separated by ˜1 cm from the connecting piece to the endotracheal tube. FiO₂ was increased to 100%. Spontaneous breathing and tissue oxygen saturation were monitored. Once both were sustained, an uncuffed nasal tube (Murphy tube, 3.0-3.5 mm ID) was inserted into one nasal passage. The tube's tip reached the mid-length of the nasal cavity (5-6 cm; the nasal cavity in fetal lambs at ˜128d gestation is ˜10 cm long) (6). The nasal tube was connected to the ventilator circuit and the ventilator was placed in the high-frequency oscillation (HFO) mode, with initial amplitude 20-25 cm H₂O, mean airway pressure 12 cm H₂O, HFO frequency 8 Hz, and I:E ratio 1:1. Conventional, positive-pressure background breaths were provided to support acceptable long-term ventilation, with initial settings of rate of 10 breaths per min, inspiratory time of 1.0 sec, and peak inspiratory pressure of the sigh breath of 25 cmH₂O.

Oxygenation was targeted for 88-94% saturation (PaO₂ 60 to 80 mmHg) by adjusting FiO₂. Ventilation was targeted for PaCO₂ between 45 to 60 mmHg by adjusting HFO amplitude and peak inspiratory pressure of the sigh breath. Lidocaine (1% solution; Hospira, Inc., Lake Forest, IL) was applied along the nostril to minimize pain and discomfort from the uncuffed nasal tube. The lambs were maintained on noninvasive respiratory support for ˜3d because of episodes of ineffective spontaneous respirations or episodes of apnea. When necessary, the lambs were stimulated to overcome ineffective spontaneous respiration. To overcome apneas, the lambs were stimulated and, if required, re-intubated. When re-intubated, the weaning process was repeated.

Orogastric feeding of ewe's colostrum (Kid & Lamb Colostrum Replacement, Land O Lakes, Arden Hills, MN) was started at ˜3 h of postnatal life (3 mL). The volume was gradually increased as tolerated, with a target over the first week of postnatal life of ˜60-90 kcal/kg/d. Parenteral dextrose was infused to maintain plasma glucose between 60 and 90 mg/dL.

Arterial blood and free-fall urine samples were collected every 24 h and analyzed for indicators of liver and kidney function (Associated Regional and University Pathologists (ARUP) Laboratories, Salt Lake City).

FPT and term reference lambs were vaccinated for Clostridium perfringens types C & D and tetanus by the veterinary staff. Vaccination removed gastrointestinal problems (diarrhea). The vaccination schedule was initial dose at 1 to 2 weeks' postnatal age for FPT lambs and at about 24 hours' postnatal age for term control lambs, followed by booster dose 2-3 weeks later.

At DOL9-10, the PT lambs were removed from all respiratory support and moved from their heated NICU bed to a heated floor pen to let them move freely (3, 4). During the initial days as FPT lambs, supplemental O₂ (blow-by via a cone) was necessary to maintain O₂ saturation at 88-94% by pulse oximetry, particularly when they slept. Need for supplemental O₂ support was typically 2 to 4d.

Matched term lambs were raised with the FPT lambs to provide a normal postnatal developmental reference. The term reference lambs were born spontaneously at term (˜150d) gestation and stayed with their ewe for ˜24 h to take colostrum. After ˜24 h, term control lambs were separated from their ewe for the remainder of the study to live with the FPT lambs.

Nutrition for the FPT and term reference lambs was provided by bottle, with an introduction to a nipple by filling the nipple with milk to let the lambs learn to suckle. Once suckling was effective, feedings were done by bottle. Ewe's milk feedings for the second week of postnatal life were 400 mL/d (140 mL/kg/d). Subsequent weekly milk feedings were 800 mL/d (220 mL/kg/d) from DOL14-20, 1,200 mL/d (240 mL/kg/d) from DOL21-30, and 1,800 mL/d (250 mL/kg/d) from DOL31-40. Milk feedings from DOL41 to 2 months corrected postnatal age were 2,700 mL/d (210 mL/kg/d), with no more than 450 mL/feeding to avoid feeding intolerance. Solid food (alfalfa pellets and hay) was introduced at ˜30d corrected postnatal age. Solid food and water were accessible ad libitum. All FPT and term reference lambs were weighed daily. The lambs were exercised daily.

Clinical Biochemistry. Plasma and urine samples were analyzed by Associated Regional and University Pathologists (ARUP) at the University of Utah. Plasma renal function parameters are urea nitrogen and creatinine. Plasma hepatic function parameters are alkaline phosphatase, aspartate aminotransferase, alanine aminotransferase, total protein, albumin, bilirubin total, and direct bilirubin. In summary, the clinical parameters in the AVR-48 treated lambs were within the normal range for lambs.

Plasma pharmacokinetics of different doses of AVR-48 via iv dosing in preterm lambs. For dose comparison, AVR-48 (0.1, 0.3, 1.0, 3.0, or 9 mg/kg/dose, iv) was given every 12 h for 7d. Pharmacokinetics (PK) evaluation for AVR-48 was done from 0 to 12 h after the initial dose. Maximum plasma concentration (Cₘₐₓ) occurred at 0.25 h for all tested doses, and theoretical Cₘₐₓ (concentration extrapolated to 0 hr; Co) for AVR-48 was followed by a bi-exponential decline of the drug levels indicated by its half-life (T_{1/2}), estimated between 0.435 and 0.895 h. Exposure to AVR-48 (AUC₍₀₋ₜ₎) increased in a dose-related manner over the dose range tested. The volume of distribution (V_{d}) for AVR-48 was estimated between 448.22 and 816.37 mL/kg, and clearance (CL) was estimated between 472.74 and 979.97 mL/hr/kg. The repeat dose Cₘₐₓ was consistent and dose-dependent.

Physiological and behavioral parameters for lambs in the early-stage study (day of life (DOL) 1-10) and late-stage study (DOL10-90). Respiratory system mechanics, neurobehavior, and physiology results for early and late-stage studies were obtained. Overall, AVR-48 improved lung function and structure, neurobehavior, and physiology.

Lung and respiratory system mechanics during the early-stage (DOL1-7) and late-stage (DOL60) studies. During the early-stage study, lung resistance and dynamic lung compliance were measured by the Drager Babylog VN500 ventilators when the PT lambs were supported by invasive mechanical ventilation (DOL1-7). No statistically significant differences between the two groups were detected in lung resistance or dynamic compliance. However, dynamic lung compliance was consistently lower in AVR-48 PT lambs than vehicle lambs.

Respiratory system mechanics during the late-stage study (DOL60). The forced oscillometry technique, using a Tremoflo N-100 unit, assessed resistance and reactance of the respiratory system on DOL 60. No significant difference was found between the groups.

Neurobehavioral outcome for the late-stage study (DOL60). The inventors assessed neurobehavior using three tests: curiosity, socialization, and memory. As shown in FIGS. 11A to 11D, AVR-48 improved neurobehavioral outcomes in FPT lambs in the late-stage study at 1 moC PNA (day of life 60).

FIGS. 11A to 11D show that AVR-48 improved neurobehavioral outcomes in FPT lambs in the late-stage study at 1 moC PNA (day of life 60). Tests were for curiosity, socialization, learning, and memory. FIG. 11A-11C) Curiosity and socialization outcomes are time at a novel object, dull (non-reflective) surface, and mirror (reflective surface) for vehicle-treated (n=3), AVR-48 (3.0 mg/kg/dose, n=4), and term reference lambs (n=6). (FIG. 11A-11C). No differences were detected at 1 moC PNA among the groups. Learning and memory outcomes for the maze test, with a milk bottle reward (FIG. 11D). Term reference lambs were never ventilated. Results presented as an average of two 10-min trials/group±SD. One-way ANOVA, Dunnett's multiple comparison test, GraphPad Prism v10.4.

Heart rate and blood pressure during the early-stage study (DOL 1-7). A statistical difference was detected for higher systolic blood pressure in the AVR-48 PT group on the DOL 6 compared to the vehicle control PT group, which was not observed on any other days.

Temperature and ventilator rate during the early-stage study (DOL 1-7). No statistical difference was detected for the temperature or respiratory rate between the AVR-48 and vehicle control PT groups.

Indices of oxygenation during the early-stage study (DOL 1-7). No statistical difference was detected for the FᵢO₂ and PaO₂ between the AVR-48 and vehicle control PT groups.

Indices of ventilation requirement during the early-stage study (DOL 1-7). No statistical difference was detected for the PIP and PaCO₂ between the AVR-48 and vehicle control PT groups.

Plasma pH, bicarbonate, base excess (BE) during the early-stage study (DOL 1-7). No statistical difference was detected for the plasma pH, bicarbonate, and BE between the AVR-48 and vehicle control PT groups.

Plasma electrolytes during the early-stage study (DOL 1-7). No statistical difference was detected for the plasma electrolytes (K⁺, Na⁺, Ca²⁺ and Cl⁻) between the AVR-48 and vehicle control treated PT groups.

Plasma glucose, hematocrit (Hct), total serum protein (Tsp), and lactate during the early-stage study (DOL 1-7). No statistical difference was detected for the plasma glucose, Hct, Tsp, and lactate between the AVR-48 and vehicle control-treated PT groups.

Enteral feeding during the early-stage study (DOL 1-7). No statistical difference was detected in the volume of tolerable enteral feeding and residual in the stomach between the AVR-48 and vehicle control-treated PT groups.

Hematology during the early-stage study (DOL 1-7). No statistical difference was detected for the volume of tolerable enteral feeding and residual in the stomach between the AVR-48 and vehicle control-treated PT groups.

ELISA, RT PCR, and Western analyses.

Effect of AVR-48 treatment on total protein and TNF-α concentrations in bronchoalveolar lavage fluid (BALf) from PT and FPT lambs at the end of the early-stage study. Increased total protein in BALf represents increased vascular permeability and pulmonary edema status of the lung. The inventors observed that AVR-48 treatment decreased total protein concentration compared to vehicle-treated PT lambs at 10 days; however, no difference was observed in the BALf from FPT lambs at 90 days. No significant change in TNF-α concentration was observed at both early and late-stage studies.

Effect of AVR-48 treatment on mRNA levels of TLR4 and cytokines in PT and FPT lamb lungs. No differences were found in relative mRNA levels between the AVR-48 and control PT lambs and between the three groups of FPT and term lambs.

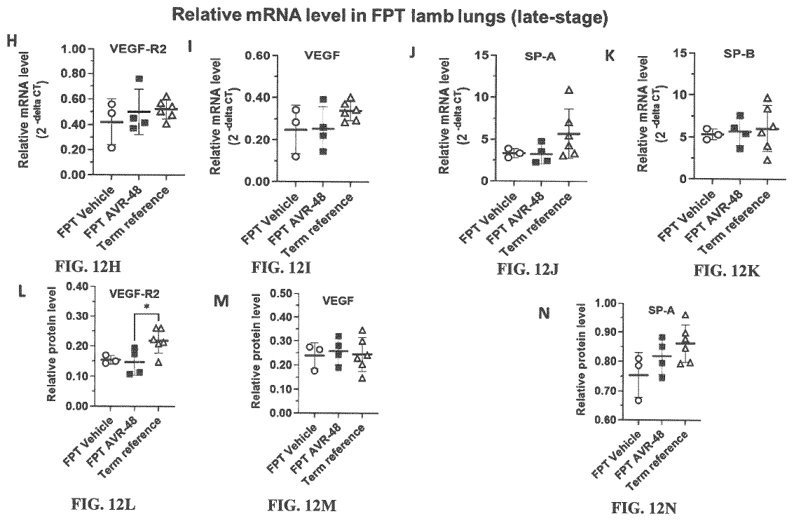

Permeability/angiogenesis markers and surfactant proteins in lung tissue homogenates after AVR-48 treatment at early and late-stage studies. Here, the mRNA and protein levels of the ligand VEGF-A and its receptor VEGFR2 were assessed, which is involved in BPD pathology and increases angiogenesis and vascular permeability in the lungs. In the early-stage study, at 10d, a significant decrease in the VEGF-A protein level, which is considered a marker for capillary permeability and angiogenesis, was detected in the AVR-48 PT group compared to the vehicle PT groups (FIG. 12F).

FIGS. 12A to 12N. mRNA and protein levels of surfactant proteins and permeability markers in PT and FPT lamb lung homogenates. In the early-stage study, at 10d, VEGF-A protein level was significantly lower in AVR-48 PT group than vehicle PT groups (FIG. 12F). Otherwise, no significant difference was detected between the two groups of PT lambs for other markers. For the late-stage study, at 90d, no significant difference was detected between FPT vehicle and FPT AVR-48 lamb lungs, or between those groups and the term reference lamb lungs for both mRNA expression and protein level for VEGF-A, VEGFR2, and SP-B. VEGF-R₂ protein level was significantly greater in the term reference group compared to both FPT groups (FIG. 12L). Data is presented as mean±SD, *p≤0.05, Student's unpaired t-test for A-G, One-way ANOVA, Dunnett's multiple comparison test for H-N. GraphPad Prism v10.4.

In a preclinical dose-finding experiment, the inventors used mechanically ventilated preterm lambs to determine that 3.0 mg/kg/dose delivered iv every 12 h was the efficacious dose of AVR-48. At this dose, AVR-48 treatment given early during ventilator/oxygen-induced lung injury in PT lambs resulted in functional improvements reflected in significantly better respiratory gas exchange parameters and respiratory severity score (RSS) that are used clinically. RSS is predictive of severe BPD in humans.³⁵ An RSS≥3.0 at postnatal d14 and an RSS≥3.6 at postnatal d21 in PT babies correlated with severe BPD or death. Pulmonary function tests revealed significantly decreased small and large airway resistance and reactance in the AVR-48 treated PT lambs compared to vehicle PT lambs. Long-term survival and growth were significantly enhanced in the FPT AVR-48 lambs compared to FPT vehicle lambs and similar to term reference lambs. These AVR-48-related improvements were accompanied by improved indices of alveolar formation early and late. These results demonstrate that AVR-48 prevented pathophysiology and histopathology of the “new bronchopulmonary dysplasia (BPD)” in a clinically relevant large-animal model. Remarkably, these improvements were accompanied by improved long-term neurobehavioral and cognitive outcomes.

Glucocorticoids are commonly used in the evolving or established phase of BPD and can improve short-term pulmonary outcomes.¹² Experimental studies in monkeys, lambs, and mice showed that both prenatal and postnatal exposures to glucocorticoids decrease the lung mesenchyme, cause thinning of the alveolus-capillary barrier, and increase the potential lung gas volume as well as increase the synthesis and secretion of surfactants.^{36,37} However, these beneficial effects are counterbalanced by fewer, larger alveoli due to the inhibition of alveolar secondary septation.^{36,37} A significant clinical concern with the use of glucocorticoids is adverse neurodevelopmental outcomes and cerebral palsy caused by steroids,¹² limiting the usefulness of glucocorticoids in treating BPD. Another concern with glucocorticoids is the decreased somatic growth demonstrated in neonatal mouse models of BPD.³⁶ In contrast with glucocorticoids, the beneficial effects of AVR-48 on respiratory gas exchange, alveolar formation, and respiratory system mechanics were achieved, including the formation of alveolar secondary septa. Similarly, the postnatal growth of FPT AVR-48 lambs was better compared with FPT vehicle control lambs. Finally, AVR-48 treatment did not compromise neurodevelopmental outcomes compared to FPT vehicle control lambs.

Persistent inflammation is an important antecedent of BPD. The inventors noted that AVR-48 significantly decreased pro-inflammatory cytokines (IL-1β, IL-6) and increased anti-inflammatory cytokines (IL-10) at specific time points during 10d of respiratory support. Clinical studies report that PT infants who developed BPD had an initial increase in IL-10 during the first 0-3d of postnatal life, followed by a decrease in IL-10 during 3-21d.³⁸ These studies demonstrate a similar pattern in the vehicle-treated PT lambs, with an increase in IL-10 level in the first 6 h of life followed by a gradual decrease over 7d. AVR-48 treatment increased IL-10 concentrations during 5-7d, augmenting the immunomodulatory activity.

Cell surface expression of CD163 on alveolar macrophages is reduced in PT infants with BPD³⁹. The transcriptomic profiling of BPD versus non-BPD PT infants demonstrated those with severe BPD had a loss of function for scavenger receptors (CD163, CD204) and mannose receptors (CD206), which is predictive of the disease.⁴⁰ CD163 is released in the circulation in its soluble form, sCD163, via cleavage of the extracellular domain by matrix metalloproteases following oxidative stress⁴¹ or via TLR4 activation after inflammatory stimuli.^{42,43} TLR4 is implicated in BPD pathogenesis.⁴⁴ The inventors found a significantly decreased TLR4 relative protein level in lung tissue (DOL 90) after AVR-48 treatment. Along with the changes in these cytokines, a relatively higher concentration of plasma sCD163 was observed in the first 4d of life in vehicle PT lambs, indicating increased inflammation. As the lambs received AVR-48, the sCD163 concentration gradually decreased.

The PT lamb model emulates the clinical context of preterm birth followed by mechanical ventilation with oxygen-rich gas for respiratory distress in a neonatal intensive care setting, including treatment with an exogenous surfactant, caffeine, and prophylactic antibiotics. The results herein show the pathophysiology and histopathology of evolving BPD,⁴⁵ long-term developmental endpoint equivalence at 36 weeks PMA in humans (term gestation in sheep), and weaning from milk at 12-18 months in humans (2 moC in sheep). The model also recapitulates challenges with inadequate early nutrition and subsequent poor weight gain, recurrent respiratory tract infections, diminished respiratory system mechanics, increased airway hyperreactivity,^{18,46} and neurodevelopmental impairment. Finally, male sex is an independent risk factor for developing BPD,⁴⁷⁻⁴⁸ which is more frequent for male lambs.^{14,17}

To conclude, the results demonstrate the novel physiological, morphological, biochemical, and neurobehavioral insights into the impact of AVR-48 on lung and neurodevelopmental outcomes early and late in the postnatal life of PT lambs. The use of AVR-48 led to increased survival and growth of the lambs. Furthermore, the sustained improvement in pulmonary outcomes was concurrently associated with long-term neurobehavioral and cognitive outcomes.

### Example 2. Anti-inflammatory and Immunomodulatory Activity of AVR-48 in Human Cord Blood Mononuclear (CBMC) and Adult Peripheral Blood Mononuclear Cells (hPBMC)

Bronchopulmonary dysplasia (BPD) is a chronic respiratory disease in premature infants due to an aberrant reparative response in developing lungs, including damage from supplemental O₂ ventilation.^{1,2}. The inventors have developed AVR-48, a small molecule immunomodulator utilizing the TLR4 pathway, for preventing BPD in at-risk preterm infants.^{3,4} AVR-48 has been studied in pre-term lamb models and currently in clinical development. This example shows that the mechanism of action (MoA) of AVR-48 promotes a robust yet “soft” innate immune response while overcoming tolerance and priming immunity for the prevention of BPD.

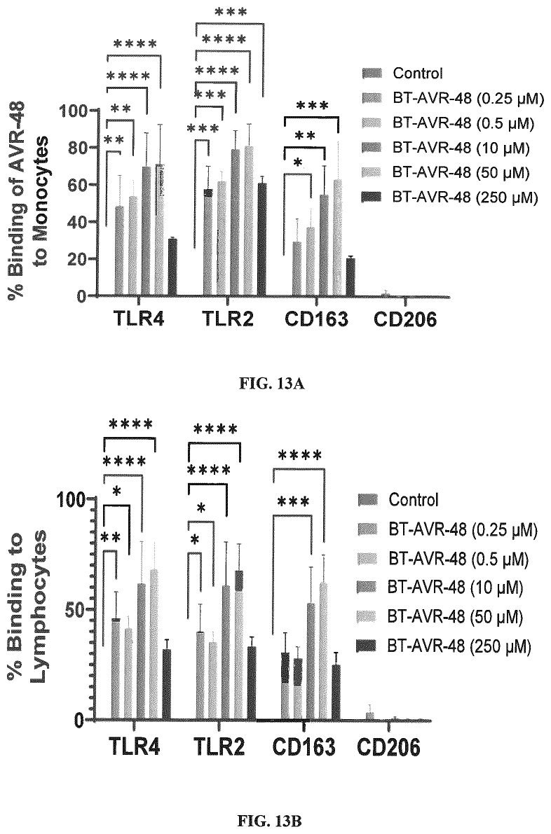

FIGS. 13A and 13B show that AVR-48 binds to TLR4 and CD163. CBMCs treated with Biotinylated-AVR-48 [100 μM] exhibit a competitive & dose-dependent binding to TLR2 and TLR4 in (FIG. 13A) monocytes and (FIG. 13B) lymphocytes as determined by flow cytometry. In addition, AVR-48 exhibits a binding affinity to CD163 scavenger receptor with both immune cell types. N=3 *p≤0.05, **p≤0.01, ***p≤0.001, **** p≤0.0001. Two-way ANOVA was used for statistical analysis.

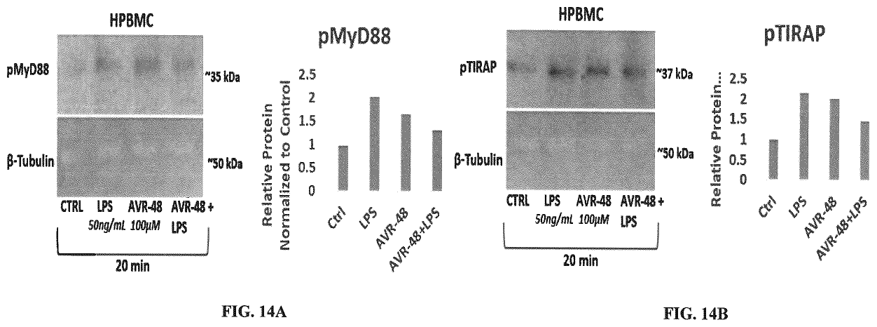

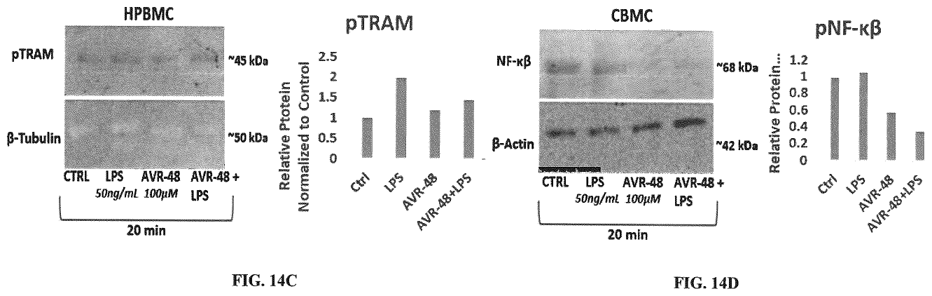

FIGS. 14A to 14D show Western Blot analysis for quantification of TLR4 pathway proteins and densitometry of (FIGS. 14A-FIG. 14C) using adult hPBMC and (FIG. 14D) CBMCs. After treatment with AVR-48 [100 μM], LPS [50 ng/mL], and in combination indicates AVR-48 activates TLR4 pathway via increasing phospho-MyD88 and phospho-TTRAP, with no activation via TRAM, and inhibiting the phosphorylation of the NF-kB pathway.

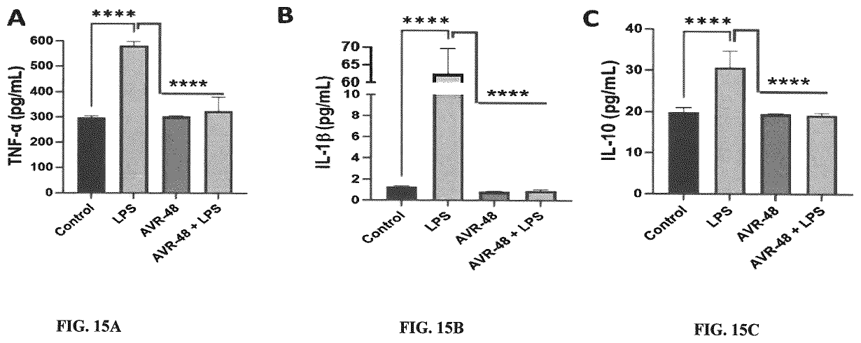

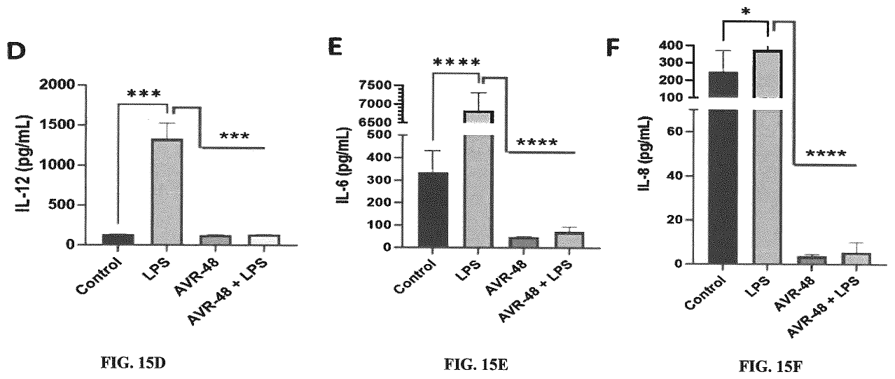

FIGS. 15A TO 15F show the AVR-48 cytokine profile in CBMCs. Cells treated with AVR-48 [100 μM], LPS [50 ng/mL] and AVR-48+LPS show a decrease (˜2-4-fold) in cytokine signal in (FIG. 15A) TNF-α, (FIG. 15B) IL-1β, (FIG. 15C) IL-10, (FIG. 15D) IL-12, (FIG. 15E) IL-6, and (FIG. 15F) IL-8, and indicates a reduced anti-inflammatory response as compared to LPS treated cells solo. N=3, One-way ANOVA was used for analysis.

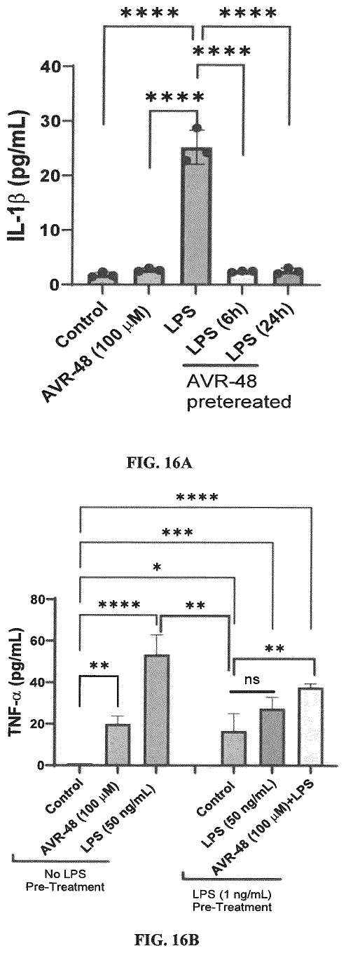

FIGS. 16A and 16B show that AVR-48 overcomes LPS-tolerance in CBMCs. Cells were dosed with AVR-48 [100 μM], LPS [50 ng/mL], and in combination show (FIG. 16A). Pre-treated CBMC with AVR-48 for 48 h followed by LPS show inhibited IL-1β profile. N=3, One-way ANOVA was used for analysis. FIG. 16B shows pretreatment of CBMC with low dose of LPS for 48 h followed by second LPS dose while didn't increase TNF-α indicating LPS tolerance, addition of AVR-48 did overcome the LPS tolerance and stimulate innate immune response via increased production of TNF-α. N=3, Two-way ANOVA.

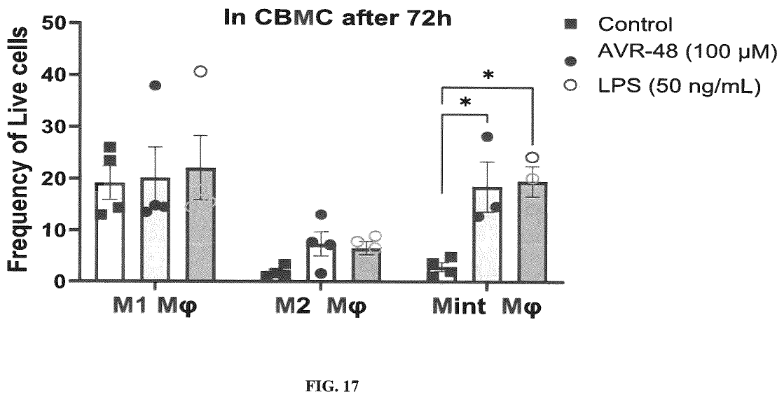

FIG. 17 shows that, AVR-48 induced intermediate macrophage lineage. CBMCs treated with AVR-48 [100 μM] and LPS [50 ng/mL] for 72 h (3d) exhibit a preference for intermediate macrophage lineage (HLA-DR^{low}/CD163^{high}) which suggests AVR-48 unique antigen-presenting cell (APC) capacity and immune action. N=3, *P<0.05, Two-way ANOVA was used for statistical analysis.

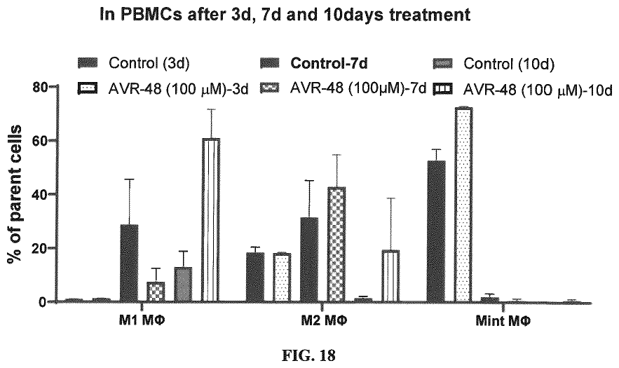

FIG. 18 shows that, AVR-48 [100 μM] treatment to hPBMCs induced highest % of intermediate macrophage (IFN-Y^{high}CD206^{high}) lineage at day 3 while polarizing to M2 phenotype (IFN-Y^{low}CD206^{high}) at day 7 and finally to M1 (IFN-Y^{high}CD206^{low}) at day 10 indicating induction of a “soft” immune modulatory response (no IL-10 via NLRP3) facilitating quick innate response and resolve.

It was found that AVR-48 binds to monocytes and lymphocytes on the TLR2/4 and CD163 receptors on CBMCs and PBMCs. AVR-48 promotes monocytes to an APC-specific intermediate macrophage lineage suggesting a unique AVR-48 mechanism. AVR-48 initiates a non-canonical TLR4 pathway (Myd88/TIRAP) promoting effective innate response in CBMCs. CBMCs treated with AVR-48 do not initiate any overt innate immune response or increased cytokines compared to LPS. AVR-48 is a novel immunomodulatory molecule that can prime and overcome immune tolerance for the prevention of BPD.

### Example 3. Novel Dual TLR2/4 Inhibitor Decreases Inflammatory Cytokines in Cord Blood Cells and Cytotoxic CD8⁺ T Cells in Murine Model of Retinopathy

Retinopathy of Prematurity (ROP) affects>32,000 preterm babies/year worldwide.^{1.} Anti-VEGF intravitreal injection therapy is commonly used but has substantial side effects.^{2.} AVR-123 (Compound 3) is macrophage modulator and partial Toll-like receptor 2/4 (TLR2/4) antagonist³ with anti-inflammatory, anti-angiogenic, and anti-fibrotic activites.^{4-7.} The present inventors show the anti-inflammatory, anti-tolerance activities and downstream TLR2/4 signaling for AVR-123 in vitro in cord blood mononuclear cells (CBMCs) and in vivo in immune cell populations of Oxygen Induce Retinopathy (OIR) mouse pups.

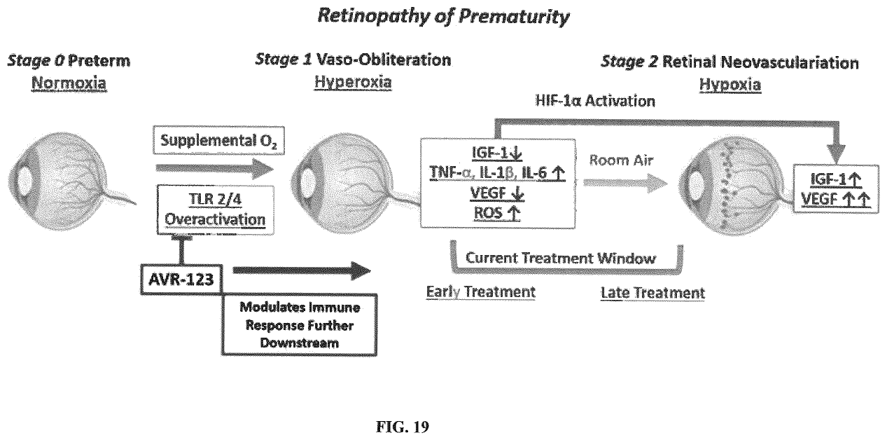

FIG. 19 shows a schematic of ROP pathology and AVR-123's role in preventing/treating ROP

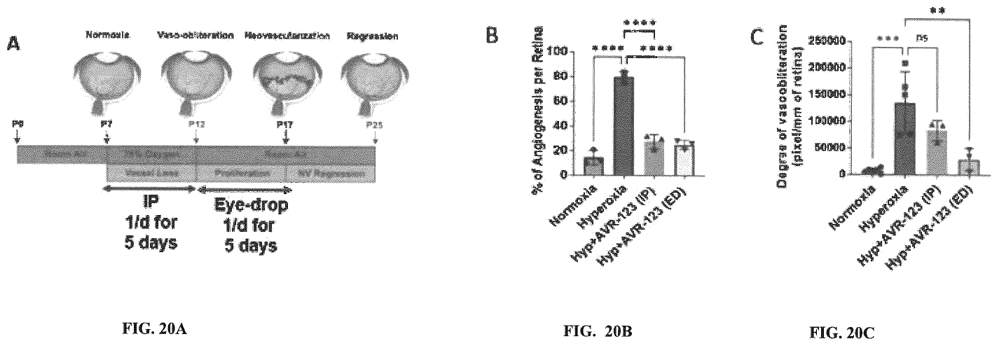

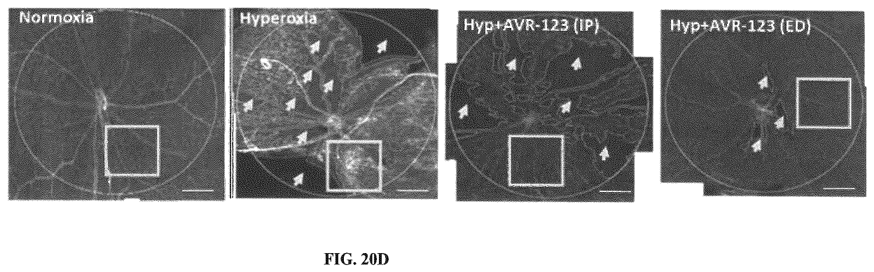

FIGS. 20A to 20D show that AVR-123 decreased vaso-obliteration and angiogenesis in mouse model of OIR. As shown in FIG. 20A schematic, AVR-123 was dosed once-a-day via IP injection (10 mg/kg) during P7-P12 (hyperoxia stage) or eye drop (AVR-123NP, 1% nanosuspension) for 5 days consecutively (P12-P17, room air, hypoxia stage). At P18 mouse pups are sacrificed and retina flat mounts were prepared. AVR-123 decreased area of Vaso obliteration via IP injection (FIG. 20B) via eye drop (FIG. 20C). The retina was stained with isolectin to visualize the blood vessels (FIG. 20D). Image J, N=3-5 retina.

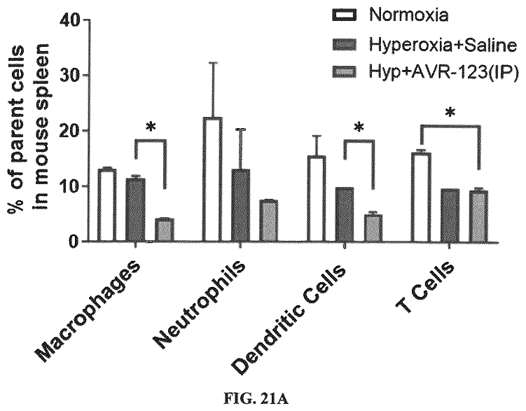

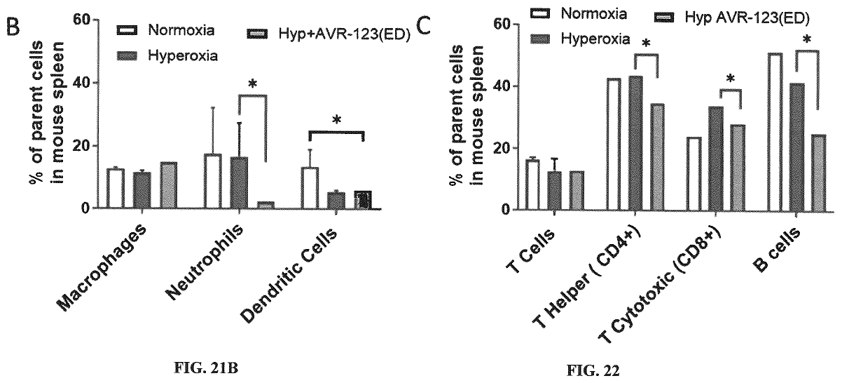

FIGS. 21A to 21C show the effect of AVR-123 treatment on mouse splenic immune cells from the OIR mice. (FIG. 21A) The splenic immune cell populations from OIR mice injected IP with AVR-123 (P7-P12) were collected at P18, and the cells (n=3 mouse spleens) were stained with immune cell-specific antibodies and analyzed via flow cytometry. (FIGS. 21B, 21C) The splenic immune cell populations from OIR mice treated with AVR-123 (ED) during P12-P17. Treatment with AVR-123 reduced populations of macrophages (F4/80+CF11b+), CD8+ T cells (CD3+CD8+), neutrophils (Ly6G+CD11b+), and dendritic cells (CD11c+MHCII+) in mouse pup spleens compared to the hyperoxia mouse group. n=3. * p<0.05, t-test.

FIG. 22 shows the cytokines in the mouse retina after OIR injury. Mice retinae treated with AVR-123NP were isolated at day 18 and RNA was extracted followed by cDNA synthesis. qPCR was performed to assess changes in the following genes: iNOS, VEGF, TNFα, TGFβ2, IGF-1, IL-1β, IL-6, and IL-10. β-actin was used for housekeeping. There was significant increase in IL-1β, TNF-α, and iNOS in the OIR retinas and after treatment with AVR-123NP, these genes were significantly downregulated. There is no significant change in VEGF or IGF1 in both hyperoxic and treated group indicating VEGF independent activity of AVR-123. N=3-5

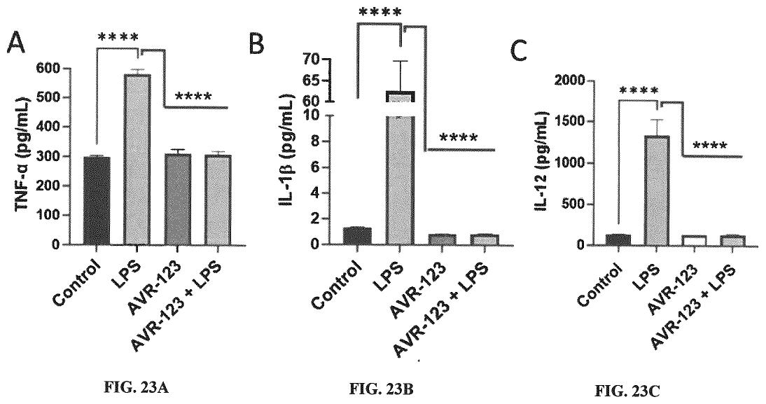

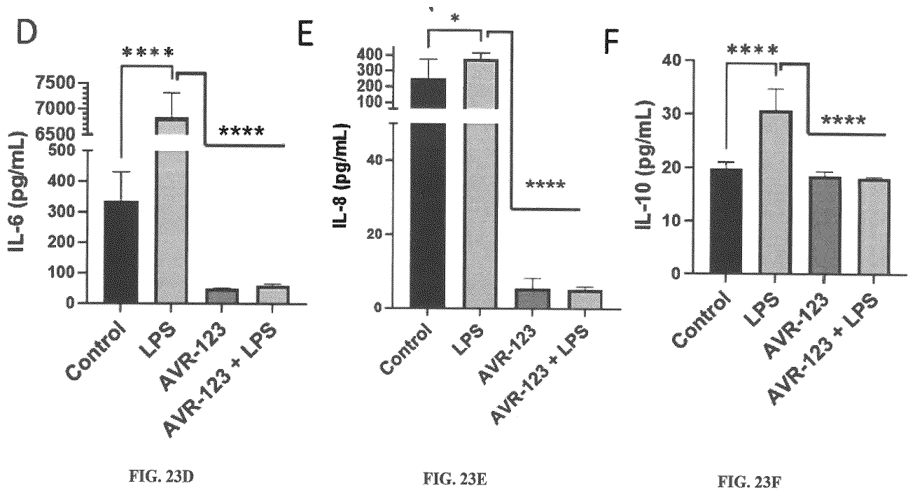

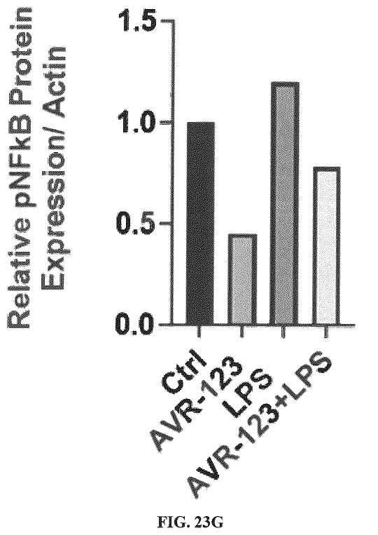

FIGS. 23A to 23G shows AVR-123's anti-inflammatory activity in a LPS challenge model in CBMC by inhibiting NFkB phosphorylation. CBMCs were treated with either AVR-123 [100 μM], LPS [50 ng/mL] or AVR-123+LPS for 24 h. AVR-123+LPS showed a decrease (˜2-4-fold) in cytokine concentrations for (FIG. 23A) TNF-α, (FIG. 23B) IL-1β, (FIG. 23C) IL-12, (FIG. 23D) IL-6, (FIG. 23E) IL-8, and (FIG. 23F) IL-10, compared to LPS treated cells. (FIG. 23G) AVR-123 decreases phosphorylated NFkB alone and when combined with LPS in CBMCs via western blot analysis. N=3, One-way ANOVA.

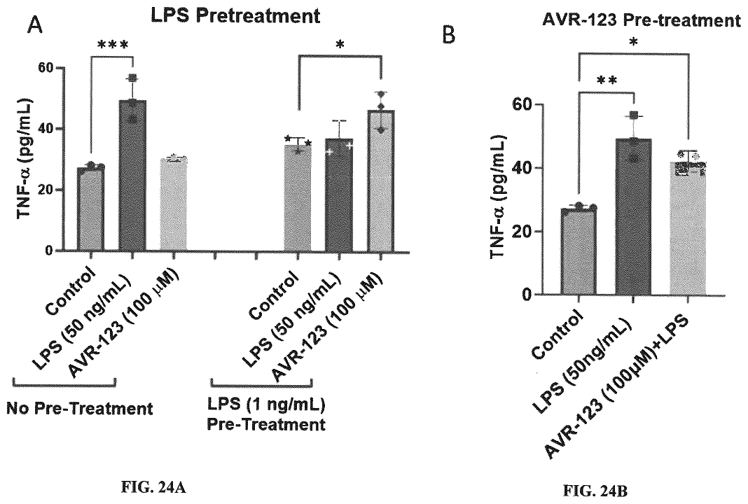

FIGS. 24A and 24B show that LPS tolerance is reversed with AVR-123 treatment. (FIG. 24A) Low dose LPS (1 ng/mL) pre-treatment for 48 h followed by high dose (50 ng/mL) LPS for another 24 h in CBMCs showed lower level of TNF-α, an indication of tolerance where addition of AVR-123 significantly increased TNF-α. (FIG. 24B) Pretreatment of AVR-48 for 48 h followed by LPS treatment for additional 24 h, showed significant higher level of TNF-α compared to control. N=3 *p<0.05, **p<0.01, ***p<0.001. One-way ANOVA and t tests.

It was found that in a mouse model system for ROP, in mice retinae (n=6) hyperoxia induced vaso-obliteration that AVR-123 nanosuspension (NP) eye drop or IP injection significantly prevented retinal damage.

In the OIR model, AVR-123 treatment decreased macrophages, neutrophils and cytotoxic CD8+ T cells in mouse spleen after eye drop and cytokines in retinae demonstrating anti-inflammatory effect both locally and systemically.

In CBMCs, AVR-123 treatment decreased the LPS induced inflammation, and also reversed the LPS immunotolerance which is critical for premature immunity and a key factor for ROP development.

These results demonstrate that targeting the dual TLR2/4 pathway will decrease inflammation, angiogenesis, and vaso-obliteration in vitro and in vivo and decrease cytotoxic immune cells. AVR-123 has passed genotoxicity studies and preliminary pharmacokinetics is established in rat plasma via IV injection.

AVR-123 can be used as anti-angiogenic treatment in eye either alone or in combination with Anti-VEGF therapy for ROP and other retinopathy indications including Diabetic Retinopathy and neovascular age related macular degeneration, corneal angiogenesis or ocular fibrosis.

### Example Formulation

TABLE 5
Summary of ingredients used in the
preparation of the nanosuspensions.

| Ingredient | Amount (%) | Amount (mg) |
|---|---|---|
| AVR-48, -84, or -123 | 5 | 151 |
| Hydroxypropyl cellulose SSL | 2.5 | 75 |
| Sodium dodecyl sulfate | 0.1 | 3 |
| Water | | Qs ad. 3000 |

The test item, the stabilizer (HPC-SSL) and the surfactant (SDS) were accurately weighed and transferred in a 15-mL amber glass jar charged with 6 mL of yttrium-stabilized zirconia beads (0.8 mm). The suspension was brought to final weight with water in order to achieve the desired final concentration. The formulation was mixed using a vortex for at least 1 minute and then homogenized using a roller mill (Unitized Jar Mill, Model 755 RMV from U.S. Stoneware (purchased from Fisher Scientific Canada cat #08-381-1) at 50 rpm for 48 hours.

As the chemical stability of tested compounds has not been assessed, the content assay of AVR-48, AVR-84, or AVR123 was evaluated at the end of nanomilling process by HPLC.

Particles size measurement was not possible as the suspensions resulted produced clear solutions when mixed with water, dilution required prior the particle size analysis.

AVR-48 Pretreatment Increases Antigen Presenting Cells, T cells and B cells in Mouse (in vivo)

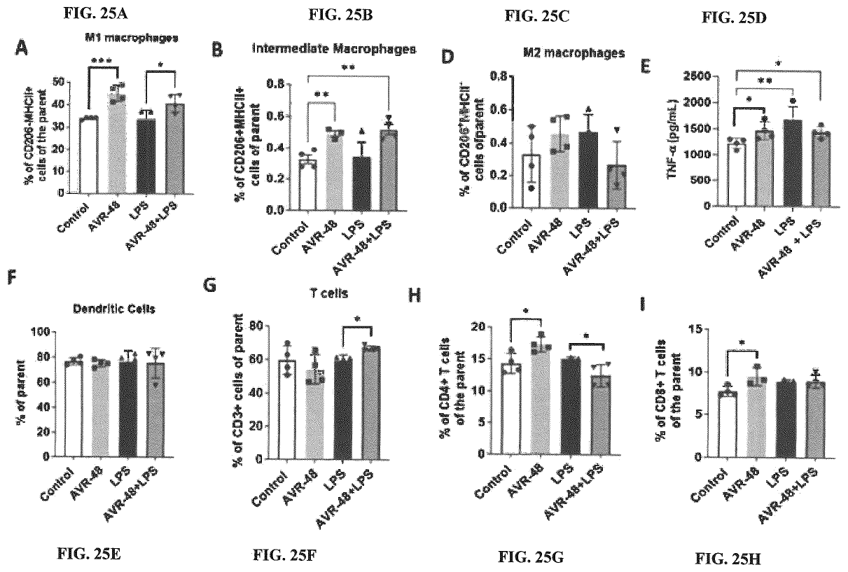

FIGS. 25A to 25G show that pretreatment of AVR-48 in mice activated the innate and adaptive immune response in the presence of LPS to demonstrate an immune modulation motif (FIG. 25A) The AVR-48 group increased M1 and (FIG. 25B) Mint macrophages as an innate immune action-reaction (FIG. 25C), whereas (FIG. 25D) M2 macrophages were not produced. AVR-48 stimulated innate and adaptive immune response via (FIG. 25E) TNF-α increase and (FIG. 25F) CD3+, (FIG. 25G) CD4+, and (FIG. 25H) CD8+ T-cells, a necessary step in adaptive response and immune resolution. *p<0.05, **p<0.01, ***p<0.001; N=4, One-way ANOVA was used for analysis.

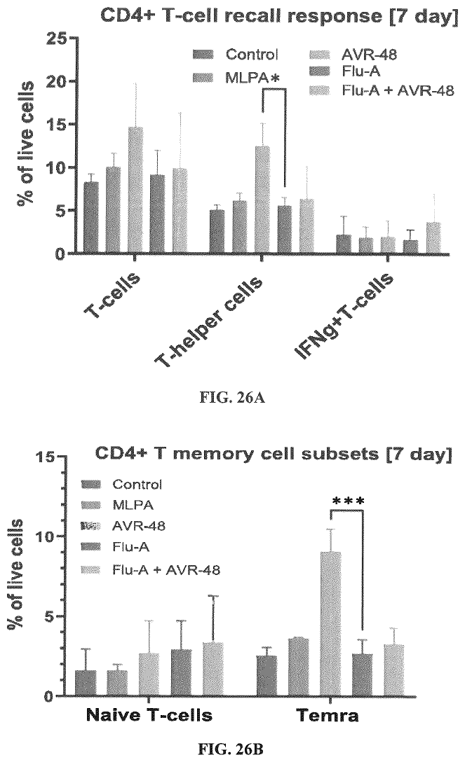

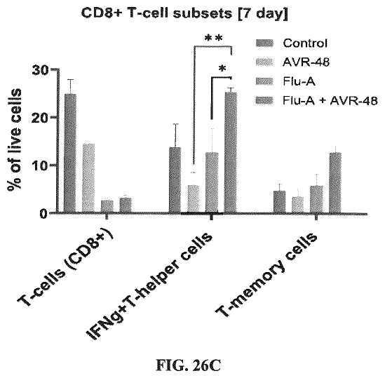

FIG. 26A to 26C show the AVR-48 induced immune profile after AVR-48 pre-dosing and dosing before LPS and MPLA insult for 7 days. (FIG. 26A) AVR-48 outperformed MPLA in CD4+T-T-cells and CD27− T-cells production. (FIG. 26B) Compared to MPLA, AVR-48 stimulated more TEMRE effector T-cell production. (FIG. 26C) AVR-48 perturbated CD8+ IFN-g+T-memory and helper cell recruitment in the presence of influenza-A peptide. N=3, *p<0.05, **p<0.01, ***p<0.001; One-way ANOVA.

AVR-48 provides a potent vaccine adjuvant by potentiating three critical immune responses salient features: (1) influx of vital helper-T cells promoting a robust adaptive immune response; (2) facilitating increased antigen/adjuvant lymph node circulation via (Th cd27⁻) and impacting access to key cells modulating the vaccine response; and (3) memory-T cell recruitment perturbating fast and efficient recall response, a strong vaccine outcome necessity.

### In Vivo Vaccine Model Studies in Mouse Models

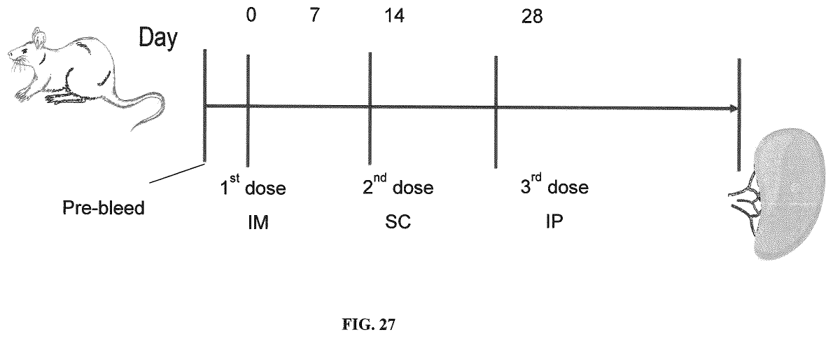

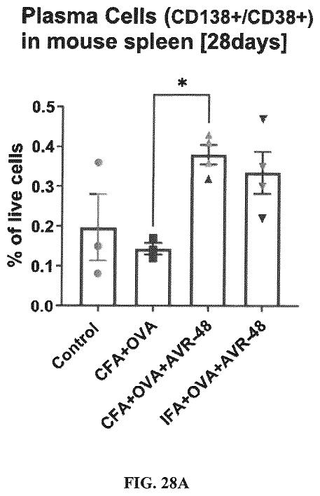

FIG. 27 shows the study design for in vivo testing. Study Design: N=4/per group (Balbc/6J, 6-7 months old). Group-1: (OVA+CFA) 1^{st} dose; (OVA+IFA) 2^{nd} dose; OVA-3rd dose; Group 2: OVA+CFA+AVR-48 1^{st} dose; OVA+IFA+AVR-48 2^{nd} dose; OVA+AVR-48 3rd dose; and Group 3: OVA+IFA+AVR48 1^{st} dose; OVA+IFA+AVR-48 2^{nd} dose; OVA+AVR-48 3^{rd} dose.

Outcome measures: Immune cell quantification (FACS) in the spleen and IgG in serum (ELISA).

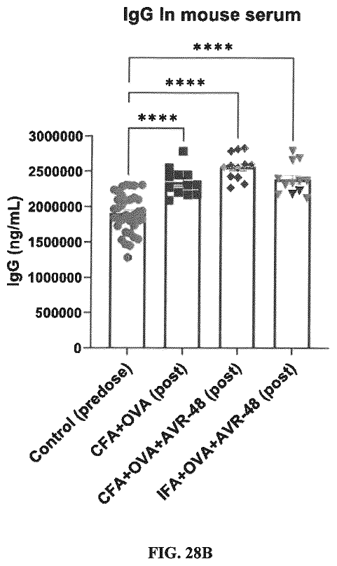

FIGS. 28A and 28B show that AVR-48 is more efficacious than Complete Freund's Adjuvant regarding plasma cell formation and IgG induction. Vaccination of AVR-48 along with Ovalbumin±CFA/IFA. Increased CD138+/CD38+/CD27+ Plasma cells in mouse spleen (FIG. 28A), and Increased IgG in serum (FIG. 28B).

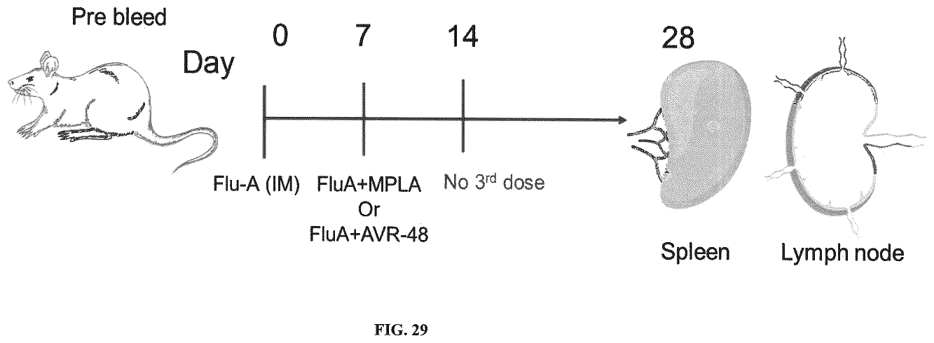

FIG. 29 shows the Mouse Model of Influenza Peptide Vaccination study design.

Study design: N=4/per group (Balbc/6J, 6-7 months old). Group-1: (FluA) 1^{st} dose; No 2^{nd} dose; Group-2: (FluA) 1^{st} dose; (FluA+MPLA) 2^{nd} dose; Group 3: AVR-48 1^{st} dose; No 2^{nd} dose; and Group 4: Flu-A 1^{st} dose; Flu-A+AVR-48 2^{nd} dose.

Outcome measures: immune cell quantification (FACS) in the spleen, and IgG in serum (ELISA).

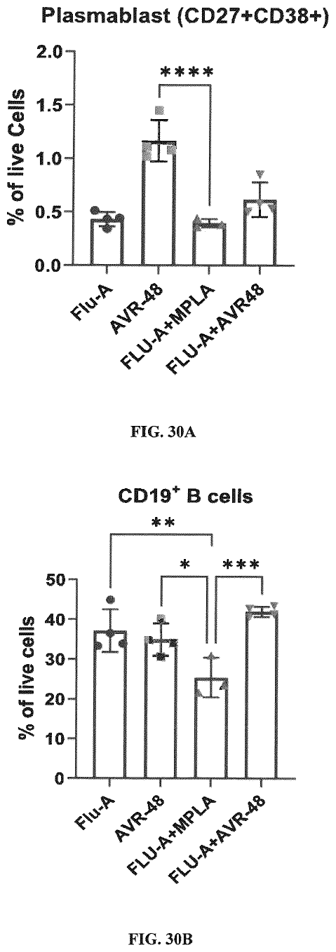

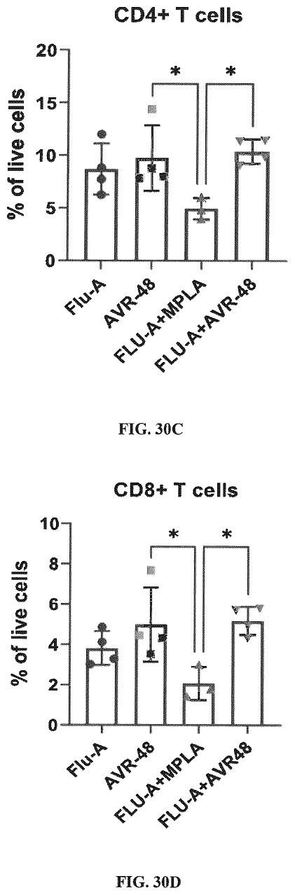

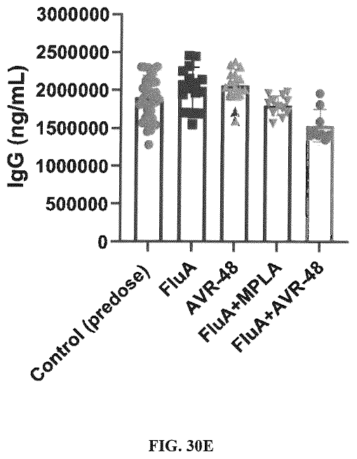

FIGS. 30A to 30E show that AVR-48 is more efficacious in improving humoral immunity than the currently used adjuvant MPLA in a mouse Flu vaccination study. Vaccination of AVR-48 alone or with Flu-A peptide increased: (FIG. 30A) CD38+/CD27+ Plasma blasts; (FIG. 30B) CD19+ B cells; (FIG. 30C) CD4+T-helper cells; (FIG. 30D) CD8+ T cells in the mouse spleen; and (FIG. 30E) increased IgG in mouse serum.

Design and Methods: This is an in vitro study. hPBMCs rested and were treated with AVR-48, LPS, MPLA, and Influenza-A peptides alone or in combination for 7 days. After staining with antibodies, cells were processed and analyzed for innate and adaptive immune surface markers via flow cytometry.

In vivo study: In one study, C57Bl/6J mice (n=4/group) were dosed intramuscularly (IM) with either complete Freund's adjuvant (CFA) or incomplete Freund's adjuvant (IFA) along with AVR-48 for a 28-day study using a prime-boost-boost regimen. In the second study, mice were dosed with MPLA and Flu-peptide using only the priming dose regimen (no booster). Splenic immune cells and plasma immunoglobin concentrations were assessed at day 28.

Human PBMCs treated with AVR-48 or AVR-48+Influenza-A peptide show increased T-helper and memory cells compared to MPLA, particularly CD27 T-cells and terminal effector memory recall (TEMRE) cells. AVR-48 enhances CD4⁺ and CD8⁺ IFN-γ⁺T⁺ memory and T-helper cells with Influenza-A peptide (FIG. 26A to 26C).

In mice, AVR-48 pretreatment boosts M1 and intermediate macrophages (Mᵢₙₜ) macrophages, increases antigen-presenting cells (APCs, T, and B cells)) after LPS insult (FIGS. 31A to 31I). In the vaccination experiments, after 28 days, IgG priming plasma cells were significantly higher in the AVR-48 group compared to CFA and ovalbumin alone (study-1) and compared to Flu-peptide and MPLA (study 2), along with increased IgG concentration (FIGS. 30A to 30E). AVR-48 increases TNF-α and M1/Mᵢₙₜ macrophages, suggesting a successful activation of the innate immune stage. In the adaptive stage, AVR-48 recruits CD4+ and CD8+, IFN-γ, helper-T cells, and memory-T cell recruitment, potentially perturbing a fast and efficient recall response.

Both hPBMCs and mice pretreated with AVR-48 alone and/or with influenza-A peptides demonstrated a significantly higher innate and adaptive immune response than MPLA. AVR-48 has the potential to be a safe and effective vaccine adjuvant.

It is contemplated that any embodiment discussed in this specification can be implemented with respect to any method, kit, reagent, or composition of the invention, and vice versa. Furthermore, compositions of the invention can be used to achieve the methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

Additionally, the section headings herein are provided for consistency with the suggestions under 37 CFR 1.77 or otherwise to provide organizational cues. These headings shall not limit or characterize the invention(s) set out in any claims that may issue from this disclosure. Specifically and by way of example, although the headings refer to a “Field of Invention,” such claims should not be limited by the language under this heading to describe the so-called technical field. Further, a description of technology in the “Background of the Invention” section is not to be construed as an admission that technology is prior art to any invention(s) in this disclosure. Neither is the “Summary” to be considered a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

### REFERENCES—EXAMPLE 1

(1) Gilfillan, M.; Bhandari, A.; Bhandari, V. BMJ (Clinical research ed.) 2021, 375, n1974.
(2) Mowitz, M. E.; Mangili, A.; Han, L.; Ayyagari, R.; Gao, W.; Wang, J.; Zhao, J.; Sarda, S. P. Expert review of pharmacoeconomics & outcomes research 2021, 21, 1117.
(3) Humayun, J.; Lofqvist, C.; Ley, D.; Hellström, A.; Gyllensten, H. BMJ open 2021, 11, e045729.
(4) van Katwyk, S.; Augustine, S.; Thebaud, B.; Thavorn, K. BMC Pediatr 2020, 20, 136.
(5) Jackson, C. M.; Demmert, M.; Mukherjee, S.; Isaacs, T.; Thompson, R.; Chastain, C.; Gray, J.; Senthamaraikannan, P.; Presicce, P.; Chetal, K.; Salomonis, N.; Miller, L. A.; Jobe, A. H.; Kallapur, S. G.; Zacharias, W. J.; Lewkowich, I P.; Deshmukh, H.; Chougnet, C. A. Mucosal immunology 2022, 15, 730.
(6) Toth, A.; Steinmeyer, S.; Kannan, P.; Gray, J.; Jackson, C. M.; Mukherjee, S.; Demmert, M.; Sheak, J. R.; Benson, D.; Kitzmiller, J.; Wayman, J. A.; Presicce, P.; Cates, C.; Rubin, R.; Chetal, K.; Du, Y.; Miao, Y.; Gu, M.; Guo, M.; Kalinichenko, V. V.; Kallapur, S. G.; Miraldi, E. R.; Xu, Y.; Swarr, D.; Lewkowich, I.; Salomonis, N.; Miller, L.; Sucre, J. S.; Whitsett, J. A.; Chougnet, C. A.; Jobe, A. H.; Deshmukh, H.; Zacharias, W. J. Science translational medicine 2022, 14, eab18574.
(7) Savani, R. C. Semin Perinatol 2018, 42, 459.
(8) Behera, S.; Panda, S. K.; Donkor, M.; Acharya, E.; Jones, H.; Acharya, S. 2022, 2, 651.
(9) Das, P.; Acharya, S.; Prahaladan, V. M.; Kumova, O. K.; Malaeb, S.; Behera, S.; Agarwal, B.; Christensen, D. J.; Carey, A. J.; Bhandari, V. International journal of molecular sciences 2021, 22.
(10) Bhandari, V. Front Pediatr 2014, 2, 76.
(11) McEvoy, C. T.; Jain, L.; Schmidt, B.; Abman, S.; Bancalari, E.; Aschner, J. L. Annals of the American Thoracic Society 2014, 11 Suppl 3, S146.
(12) Jensen, E. A.; Wiener, L. E.; Rysavy, M. A.; Dysart, K. C.; Gantz, M. G.; Eichenwald, E. C.; Greenberg, R. G.; Harmon, H. M.; Laughon, M. M.; Watterberg, K. L.; Walsh, M. C.; Yoder, B. A.; Lorch, S. A.; DeMauro, S. B. JAMA network open 2023, 6, e2312277.
(13) Shah, D.; Das, P.; Acharya, S.; Agarwal, B.; Christensen, D. J.; Robertson, S. M.; Bhandari, V. International journal of molecular sciences 2021, 22.
(14) Dahl, M. J.; Veneroni, C.; Lavizzari, A.; Bowen, S.; Emerson, H.; Rebentisch, A.; Dawson, E.; Summers, K.; Pettet, L.; Wang, Z.; Null, D. M.; Yoder, B. A.; Dellaca, R. L.; Albertine, K. H. American journal of physiology. Lung cellular and molecular physiology 2021, 321, L248.
(15) Albertine, K. H.; Dahl, M. J.; Rebentisch, A.; Dawson, E.; Nabi, A.; Bowen, S.; Miers, C.; Wang, Z.; Yang, H.; Yu, B.; Null, D. M.; Keefe, D.; Chung, J.-K.; Barton, N.; Carey, G.; Ward, B. Pediatr Res 2023, 93, 1528.
(16) Bizzotto, D.; Dahl, M. J.; Veneroni, C.; Lavizzari, A.; Rebentisch, A.; Dawson, E.; Bowen, S.; Zuspan, K.; Yoder, B. A.; Albertine, K. H.; Dellaca, R. L. American journal of physiology. Lung cellular and molecular physiology 2024, 327, L203.
(17) Dahl, M. J.; Bowen, S.; Aoki, T.; Rebentisch, A.; Dawson, E.; Pettet, L.; Emerson, H.; Yu, B.; Wang, Z.; Yang, H.; Zhang, C.; Presson, A. P.; Joss-Moore, L.; Null, D. M.; Yoder, B. A.; Albertine, K. H. Am J Physiol Lung Cell Mol Physiol 2018, 315, L816.
(18) Klinger, A. P.; Travers, C. P.; Martin, A.; Kuo, H. C.; Alishlash, A. S.; Harris, W. T.; Carlo, W. A.; Ambalavanan, N. Pediatr Res 2020, 88, 293.
(19) Crapo, R. O.; Casaburi, R.; Coates, A. L.; Enright, P. L.; Hankinson, J. L.; Irvin, C. G.; MacIntyre, N. R.; McKay, R. T.; Wanger, J. S.; Anderson, S. D.; Cockcroft, D. W.; Fish, J. E.; Sterk, P. J. Am J Respir Crit Care Med 2000, 161, 309.
(20) Coulon, M.; Hild, S.; Schroeer, A.; Janczak, A. M.; Zanella, A. J. Physiology & Behavior 2011, 103, 575.
(21) Erhard, H. W.; Boissy, A.; Rae, M. T.; Rhind, S. M. Behav Brain Res 2004, 151, 25.
(22) Lee, C.; Colegate, S.; Fisher, A. D. Appl Ani Beh Sci 2006, 96, 43.
(23) Greiveldinger, L.; Veissier, I.; Boissy, A. Physiol Behav 2007, 92, 675.
(24) Doyle, R. E.; Lee, C.; Deiss, V.; Fisher, A. D.; Hinch, G. N.; Boissy, A. Physiology & Behavior 2011, 102, 503.
(25) Hild, S.; Coulon, M.; Schroeer, A.; Andersen, I. L.; Zanella, A. J. Physiology & Behavior 2011, 104, 384.
(26) Reyburn, B.; Li, M.; Metcalfe, D. B.; Kroll, N. J.; Alvord, J.; Wint, A.; Dahl, M. J.; Sun, J.; Dong, L.; Wang, Z. M.; Callaway, C.; McKnight, R. A.; Moyer-Mileur, L.; Yoder, B. A.; Null, D. M.; Lane, R. H.; Albertine, K. H. Am J Respir Crit Care Med 2008, 178, 407.
(27) Albertine, K. H.; Dahl, M. J.; Gonzales, L. W.; Wang, Z. M.; Metcalfe, D.; Hyde, D. M.; Plopper, C. G.; Starcher, B. C.; Carlton, D. P.; Bland, R. D. Am J Physiol Lung Cell Mol Physiol 2010, 299, L59.
(28) Joss-Moore, L.; Carroll, T.; Yang, Y.; Fitzhugh, M.; Metcalfe, D.; Oman, J.; Hale, M.; Dong, L.; Wang, Z. M.; Yu, X.; Callaway, C. W.; O'Brien, E.; McKnight, R. A.; Lane, R. H.; Albertine, K. H. Pediatr Res 2013, 73, 612.
(29) Hsia, C. C.; Hyde, D. M.; Ochs, M.; Weibel, E. R. Am J Respir Crit Care Med 2010, 181, 394.
(30) Svedenkrans, J.; Stoecklin, B.; Jones, J. G.; Doherty, D. A.; Pillow, J. J. American journal of respiratory and critical care medicine 2019, 200, 471.
(31) Crapo, R. O.; Casaburi, R.; Coates, A. L.; Enright, P. L.; Hankinson, J. L.; Irvin, C. G.; MacIntyre, N. R.; McKay, R. T.; Wanger, J. S.; Anderson, S. D.; Cockcroft, D. W.; Fish, J. E.; Sterk, P. J. American journal of respiratory and critical care medicine 2000, 161, 309.
(32) Ke, X.; Xing, B.; Dahl, M. J.; Alvord, J.; McKnight, R. A.; Lane, R. H.; Albertine, K. H. Pediatric Research 2021.
(33) Albertine, K. H. Clinics in perinatology 2012, 39, 727.
(34) Unsworth, W. P.; Taylor, J. A.; Robinson, J. E. Biology of Reproduction 2005, 72, 619.
(35) Jung, Y. H.; Jang, J.; Kim, H.-S.; Shin, S. H.; Choi, C. W.; Kim, E.-K.; Kim, B. I. BMC Pediatrics 2019, 19, 121.
(36) Lok, I. M.; Wever, K. E.; Vliegenthart, R. J. S.; Onland, W.; van Kaam, A. H.; van Tuyl, M. Pediatric research 2024, 96, 1141.
(37) Willet, K. E.; Jobe, A. H.; Ikegami, M.; Kovar, J.; Sly, P. D. American journal of respiratory and critical care medicine 2001, 163, 1437.
(38) Ambalavanan, N.; Carlo, W. A.; D'Angio, C. T.; McDonald, S. A.; Das, A.; Schendel, D.; Thorsen, P.; Higgins, R. D.; Eunice Kennedy Shriver National Institute of Child, H.; Human Development Neonatal Research, N. Pediatrics 2009, 123, 1132.
(39) Kwon, J. H.; Kim, M.; Bae, Y. K.; Kim, G.-H.; Choi, S. J.; Oh, W.; Um, S.; Jin, H. J. Int J Mol Sci 2019, 20, 4815.
(40) Sahoo, D.; Zaramela, L. S.; Hernandez, G. E.; Mai, U.; Taheri, S.; Dang, D.; Stouch, A. N.; Medal, R. M.; McCoy, A. M.; Aschner, J. L.; Blackwell, T. S.; Zengler, K.; Prince, L. S. Communications biology 2020, 3, 259.
(41) Timmermann, M.; Hogger, P. Free radical biology & medicine 2005, 39, 98.
(42) Zhi, Y.; Gao, P.; Xin, X.; Li, W.; Ji, L.; Zhang, L.; Zhang, X.; Zhang, J. Molecular medicine reports 2017, 15, 2931.
(43) Weaver, L. K.; Pioli, P. A.; Wardwell, K.; Vogel, S. N.; Guyre, P. M. Journal of leukocyte biology 2007, 81, 663.
(44) Yao, L.; Shi, Y.; Zhao, X.; Hou, A.; Xing, Y.; Fu, J.; Xue, X. International journal of molecular medicine 2017, 39, 1403.
(45) Albertine, K. H. Am J Physiol Lung Cell Mol Physiol 2015, 308, L983.
(46) Voraphani, N.; Stern, D. A.; Zhai, J.; Wright, A. L.; Halonen, M.; Sherrill, D. L.; Hallberg, J.; Kull, I.; Bergstrom, A.; Murray, C. S.; Lowe, L.; Custovic, A.; Morgan, W. J.; Martinez, F. D.; Melen, E.; Simpson, A.; Guerra, S. Lancet Respir Med 2022, 10, 59.
(47) Lee, R.; Kostina, E.; Dassios, T.; Greenough, A. European journal of pediatrics 2023, 182, 1417.
(48) Lingappan, K.; Jiang, W.; Wang, L.; Moorthy, B. Am J Physiol Lung Cell Mol Physiol 2016, 311, L481.
(49) Barros, F. C.; Papageorghiou, A. T.; Victora, C. G.; Noble, J. A.; Pang, R.; lams, J.; Cheikh Ismail, L.; Goldenberg, R. L.; Lambert, A.; Kramer, M. S.; Carvalho, M.; Conde-Agudelo, A.; Jaffer, Y. A.; Bertino, E.; Gravett, M. G.; Altman, D. G.; Ohuma, E. O.; Purwar, M.; Frederick, I. O.; Bhutta, Z. A.; Kennedy, S. H.; Villar, J.; International, F.; Newborn Growth Consortium for the 21st, C. JAMA Pediatr 2015, 169, 220.
(50) Gomez-Lopez, N.; Galaz, J.; Miller, D.; Farias-Jofre, M.; Liu, Z.; Arenas-Hernandez, M.; Garcia-Flores, V.; Shaffer, Z.; Greenberg, J. M.; Theis, K. R.; Romero, R. Reproduction 2022, 164, R₁₁.

### REFERENCES—EXAMPLE 2

(2019). Bronchopulmonary dysplasia. Nature Reviews Disease Primers, 5(1), 1-23.
(2023). Editorial: Bronchopulmonary dysplasia: Latest advances. Frontiers in Pediatrics, 11, 1303761
(2021). Chitin-Derived AVR-48 Prevents Experimental Bronchopulmonary Dysplasia (BPD). International journal of molecular sciences, 22(16), 8547.
(2022) Chitin Derived Small Molecule AVR-48 Reprograms the Resting Macrophages to an Intermediate Phenotype and Decrease Pseudomonas aeruginosa Mouse Lung Infection. Immuno.; 2(4):651-670.

### REFERENCES—EXAMPLE 3

Clinical and experimental pediatrics, 65(3), 115-126. (2022)
J Clin Med. 2; 10(5):981. (2021)
Patent: Novel Immunomodulating Small Molecules (WO2020010090)
PMID: 33801185; PMCID: PMC7957879
Cell Reports 30, 2209-2224 (2020)
Scientific Reports volume 6, Article #27739 (2016)
Invest. Ophthalmol. Vis. Sci. 2023; 64(8):3501

What is claimed is:

1. A method for inducing intermediate macrophages in a subject, the method comprising:
administering to the subject a therapeutically effective amount of one or more compositions that comprise a compound of formula (I) or stereoisomer, enantiomer, tautomer or a pharmaceutically acceptable salt thereof: 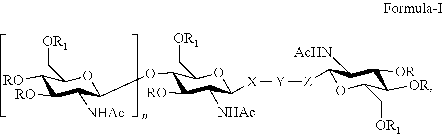
wherein n=0-5; X═NH, O, S, or CH₂; Y=Phenyl, a phenyl group substituted with at least one methyl, a phenyl group substituted with at least one nitro, a phenyl group substituted with at least one nitrogen, a phenyl group substituted with at least one boron, aryl, substituted aryl, heteroaryl, four to six membered cycloalkyl, four to six membered heterocycloalkyl; Z═NH, O, S, CH₂ or none; R═H, C(O)R₂, SO₂R₂; R₁ ═H, C(O)R₂, SO₂R₂; R₂=Ethyl, methyl, isopropyl, n-propyl, t-butyl, n-butyl, NH₂, NR₃R₄; R₃, R₄=ethyl, methyl, isopropyl, n-propyl, t-butyl, n-butyl, three to six membered cycloalkyl,
wherein the compound promotes monocyte differentiation into an antigen-presenting cell (APC)-specific intermediate macrophage lineage.

2. The method of claim 1, wherein the composition at least one of: modifies polarization of macrophages to intermediate macrophages; modifies a balance between different subtypes of macrophages toward intermediate macrophages; induces differentiation of monocytes to intermediate macrophages; or induces phenotype switching from immature macrophages to intermediate macrophages.

3. The method of claim 1, wherein the compound is selected from:

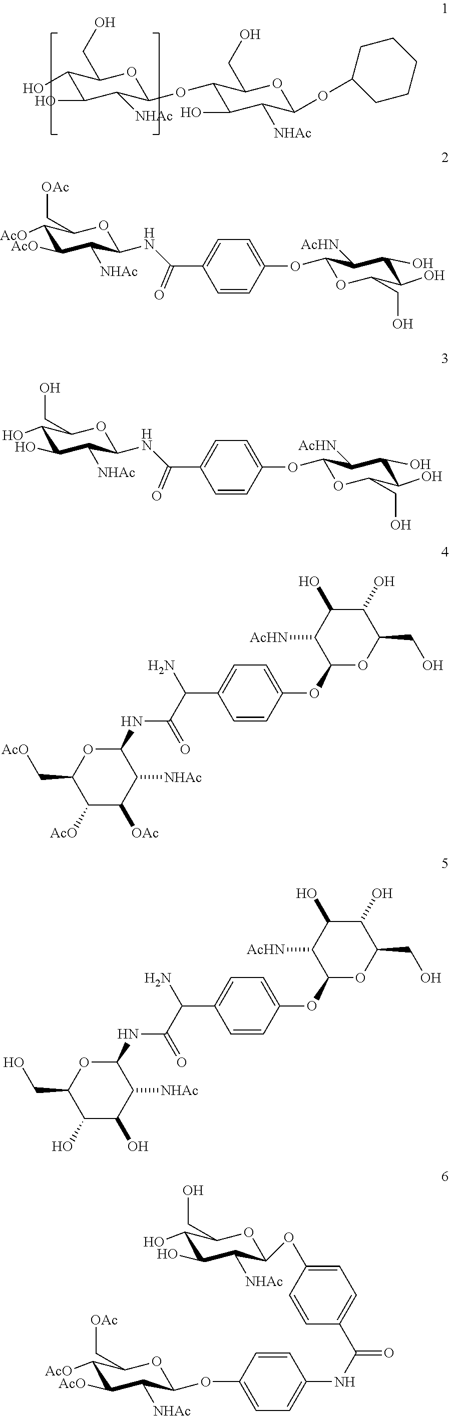

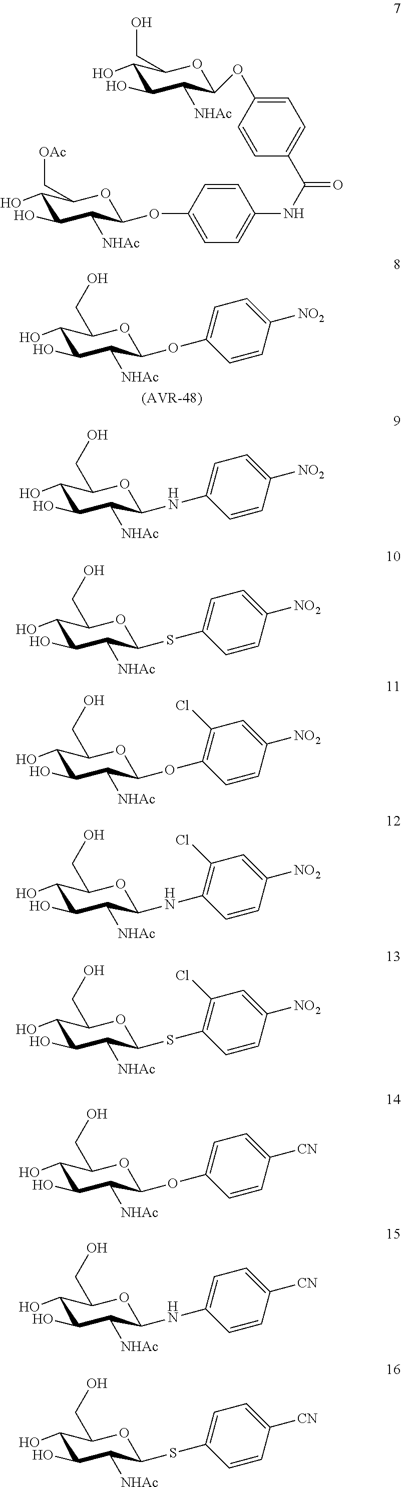

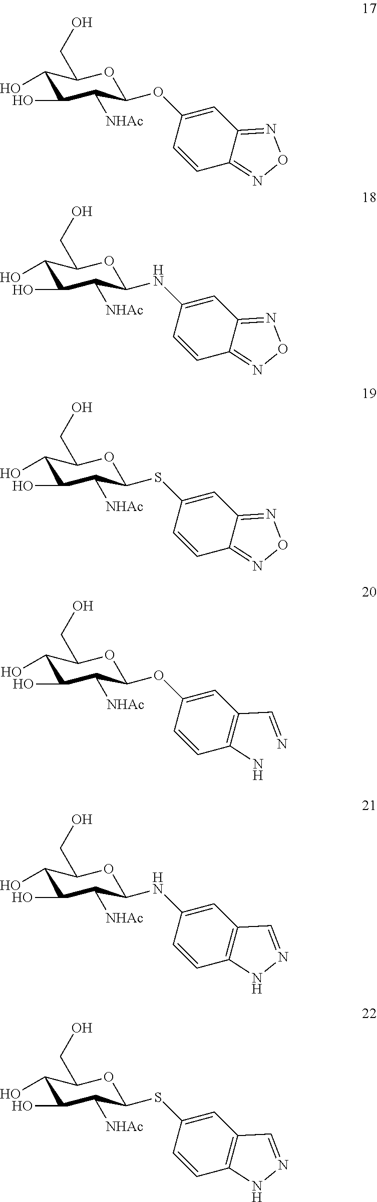

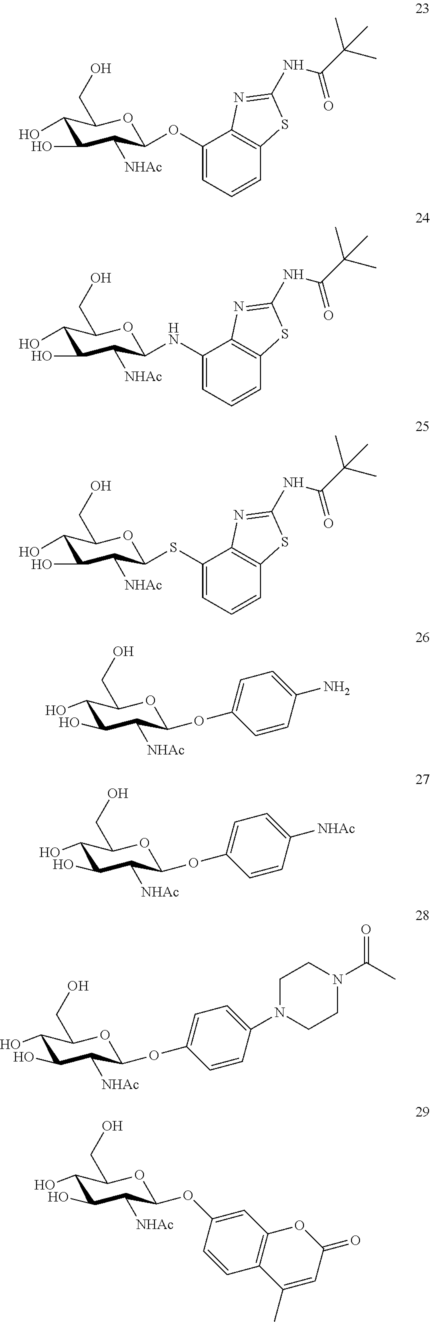

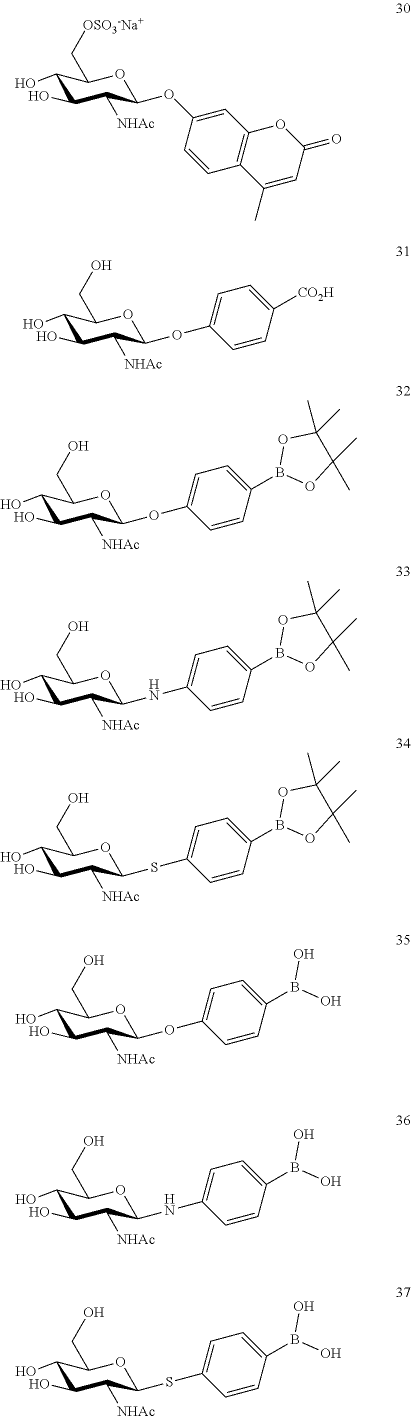

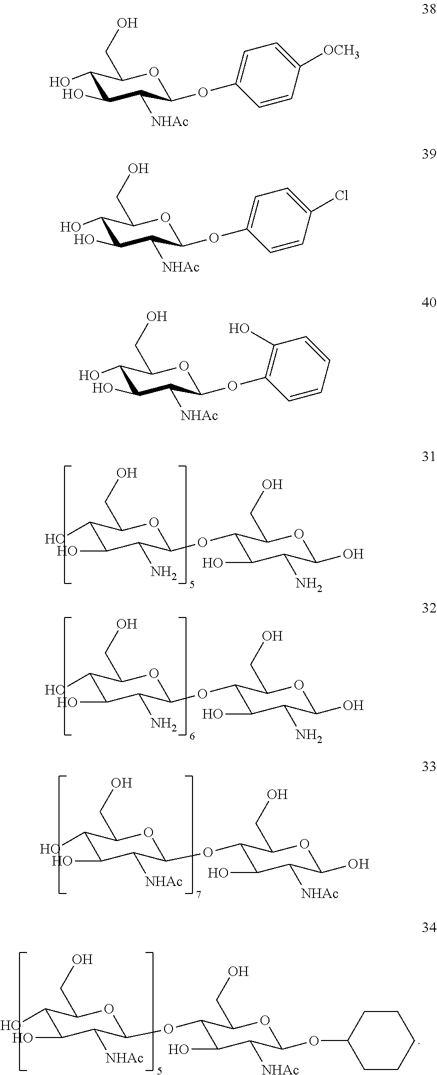

4. The method of claim 1, wherein the compound is administered by pulmonary, alveolar, enteral, parenteral, intravenous, intraperitoneal, intramuscular, subcutaneous, topical, otic, ocular, intravitreal, or oral administration.

5. The method of claim 1, wherein the compound is combined with at least one active agent selected from: amylocaine, articaine, benzocaine, bupivacaine, chloroprocaine, dibucaine, etidocaine, levobupivacaine, lidocaine, mepivacaine, metabutoxycaine, piperocaine, prilocaine, procaine, proparacaine, ropivacaine, tetracaine, corticosteroids, bronchodilators, anticholinergics, vasodilators, diuretics, anti-hypertensive agents, acetazolamide, antibiotics, antivirals, or immunosuppressive drugs.

6. The method of claim 1, wherein the compound is

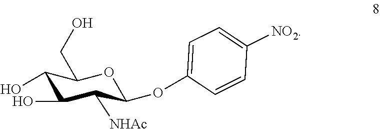

7. The method of claim 1, wherein the compound is selected from at least one of:

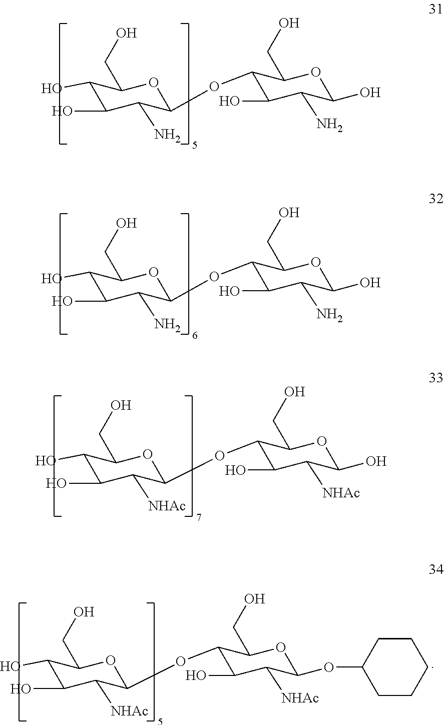

8. The method of claim 1, wherein the intermediate monocytes are HLA-DR⁺/CD163⁺.

9. The method of claim 1, wherein the compound does not bind to or trigger VEGF receptor.

10. The method of claim 1, wherein the compound binds peripheral blood mononuclear cells at both TLR4 and CD163.

11. The method of claim 1, wherein the compound decreases inflammatory cytokines in cord blood cells and CD8+ T cells in retinopathy of prematurity (ROP).

12. The method of claim 9, wherein the compound overcomes immune cell tolerance and primes immunity for prevention or treatment of bronchopulmonary dysplasia.

13. The method of claim 1, wherein the compound has at least one of: anti-inflammatory, anti-angiogenic, or anti-fibrotic activities.

14. An adjuvant comprising:
a compound of Formula I, or stereoisomer, enantiomer, tautomer or a pharmaceutically acceptable salt thereof: 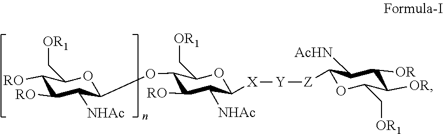
wherein n=0-5; X═NH, O, S, or CH₂; Y=Phenyl, a phenyl group substituted with at least one methyl, a phenyl group substituted with at least one nitro, a phenyl group substituted with at least one nitrogen, a phenyl group substituted with at least one boron, aryl, substituted aryl, heteroaryl, four to six membered cycloalkyl, four to six membered heterocycloalkyl; Z═NH, O, S, CH₂ or none; R═H, C(O)R₂, SO₂R₂; R₁═H, C(O)R₂, SO₂R₂; R₂=Ethyl, methyl, isopropyl, n-propyl, t-butyl, n-butyl, NH₂, NR₃R₄; R₃, R₄=ethyl, methyl, isopropyl, n-propyl, t-butyl, n-butyl, three to six membered cycloalkyl, wherein the compound promotes monocyte differentiation into an antigen-presenting cell (APC)-specific intermediate macrophage lineage.

15. The adjuvant of claim 14, wherein the adjuvant induces an increase in intermediate macrophages, B cells, T cells, and antigen presenting cells.

16. The adjuvant of claim 14, wherein the adjuvant induces an increase in at least one of CD38+/CD27+ Plasma blasts; CD19+ B cells; CD4+ T-helper cells; CD8+ T cells; or IgG.

17. The adjuvant of claim 14, wherein the composition at least one of: modifies polarization of macrophages to intermediate macrophages; modifies a balance between different subtypes of macrophages toward intermediate macrophages; induces differentiation of monocytes to intermediate macrophages; or induces phenotype switching from immature macrophages to intermediate macrophages.

18. The adjuvant of claim 14, wherein the compound is selected from:

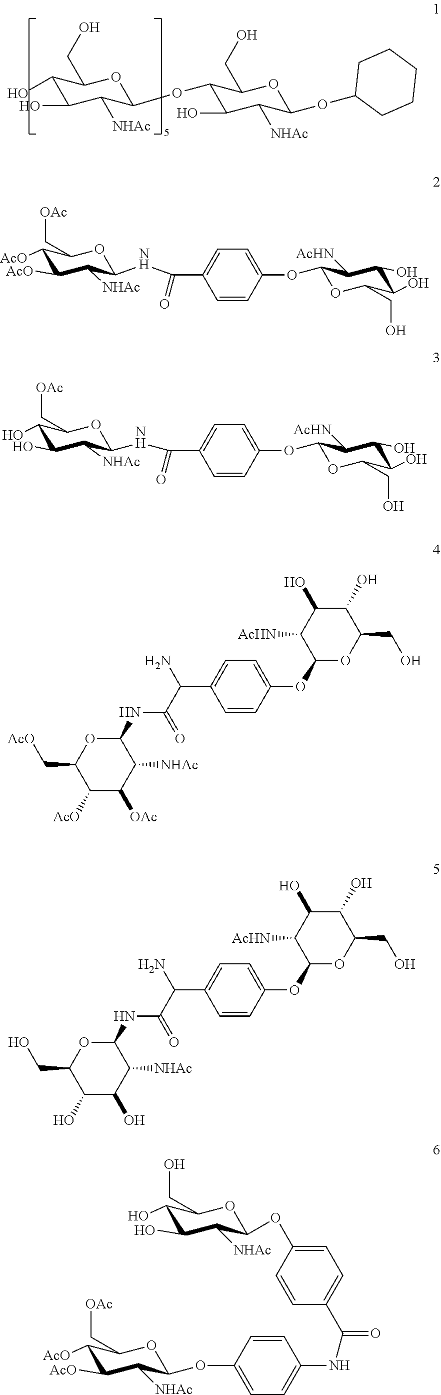

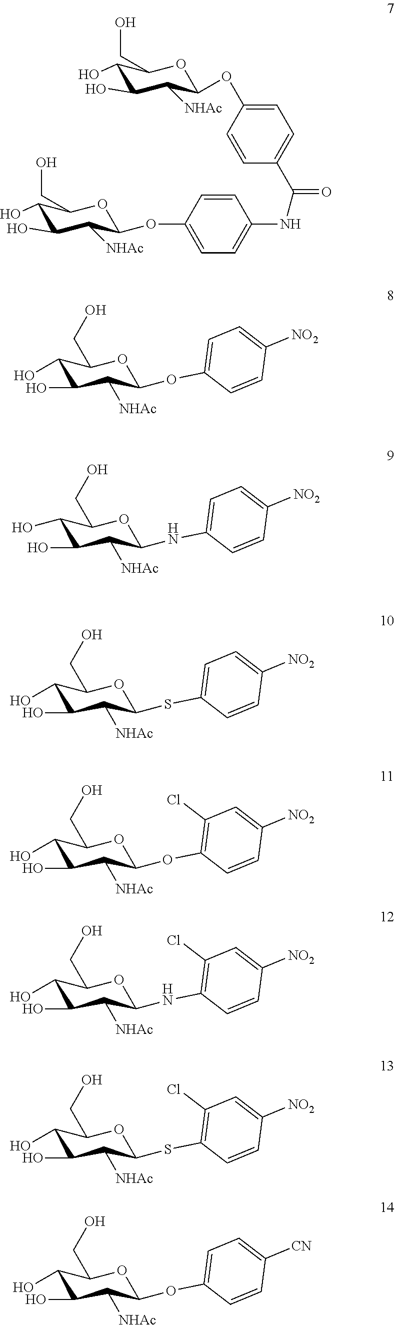

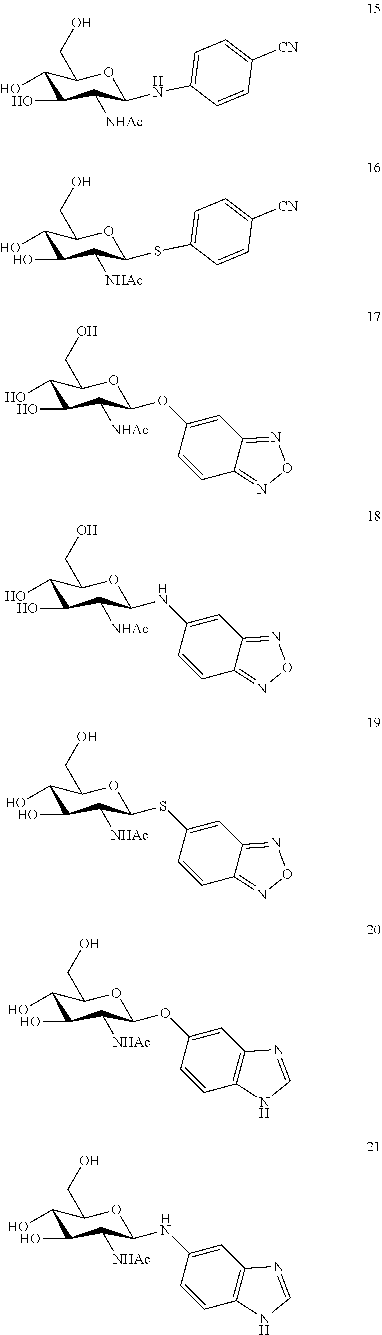

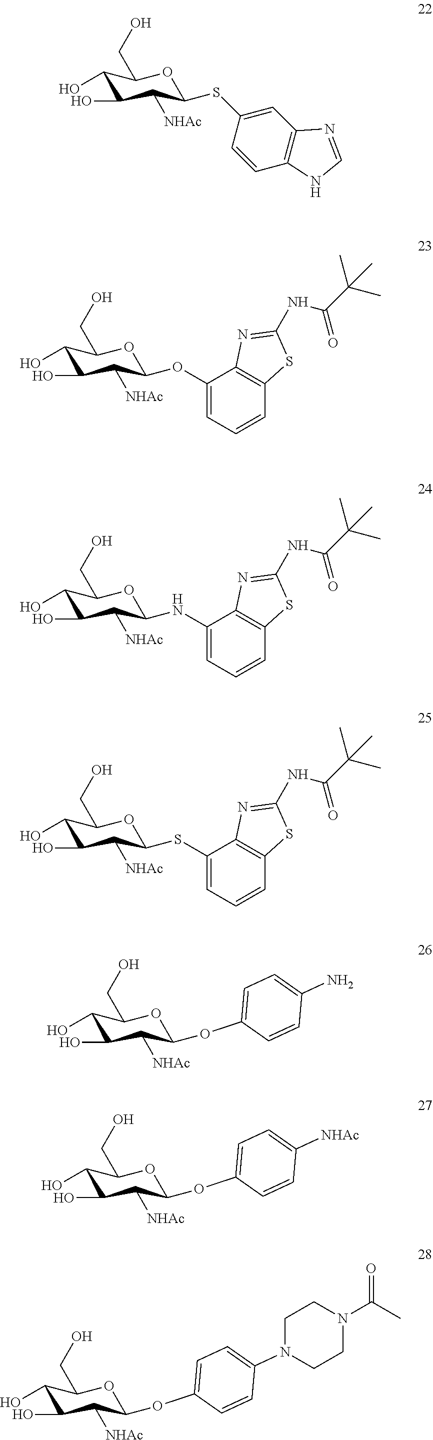

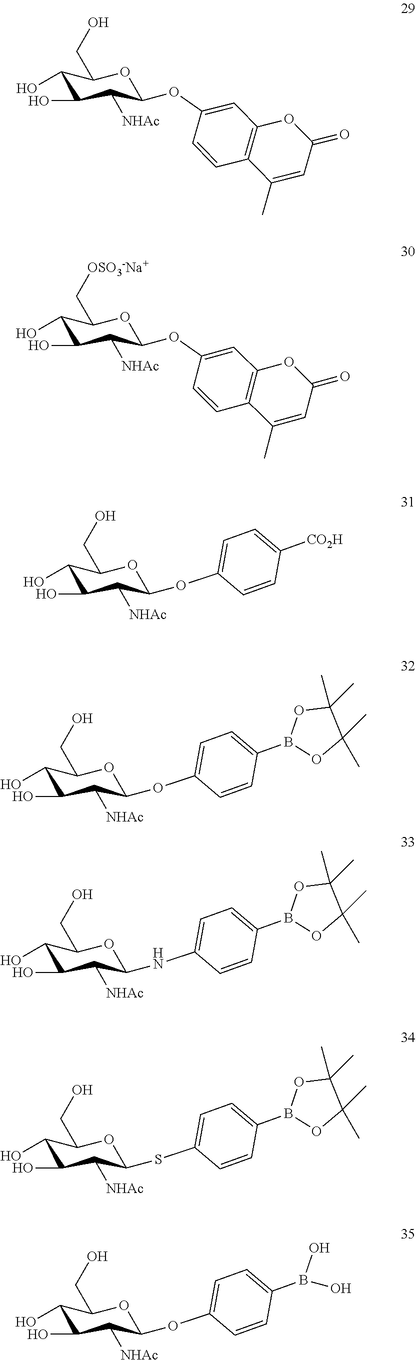

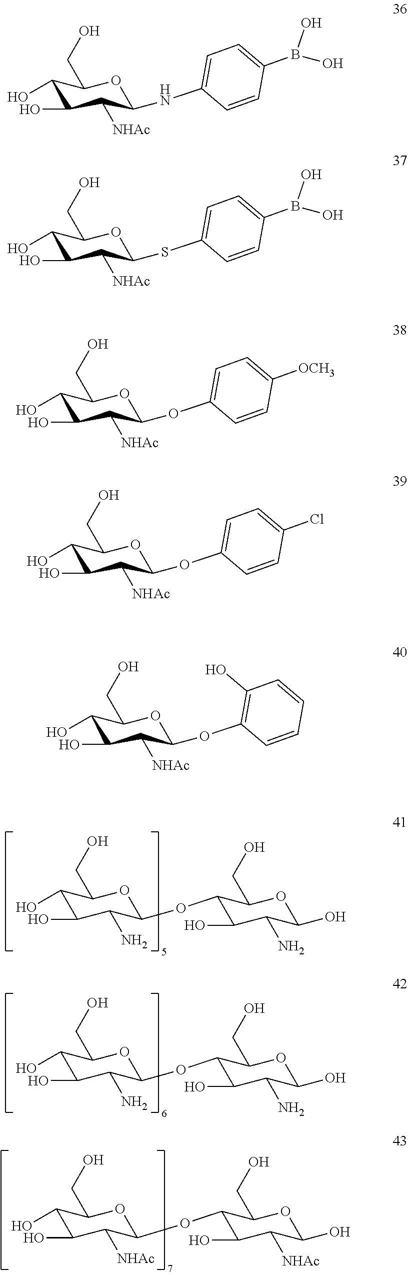

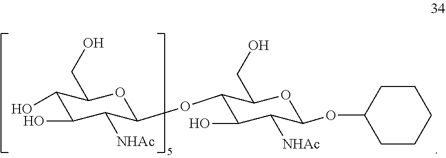

19. The adjuvant of claim 14, wherein the compound is administered by pulmonary, alveolar, enteral, parenteral, intravenous, intraperitoneal, intramuscular, subcutaneous, topical, otic, ocular, intravitreal, or oral administration.

20. The adjuvant of claim 14, wherein the compound is combined with at least one active agent selected from: amylocaine, articaine, benzocaine, bupivacaine, chloroprocaine, dibucaine, etidocaine, levobupivacaine, lidocaine, mepivacaine, metabutoxycaine, piperocaine, prilocaine, procaine, proparacaine, ropivacaine, tetracaine, corticosteroids, bronchodilators, anticholinergics, vasodilators, diuretics, anti-hypertensive agents, acetazolamide, antibiotics, antivirals, or immunosuppressive drugs.

21. The adjuvant of claim 14, wherein the compound is

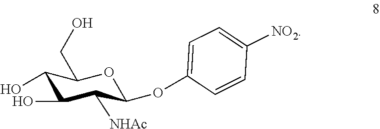

22. The adjuvant of claim 14, wherein the compound is selected from at least one of:

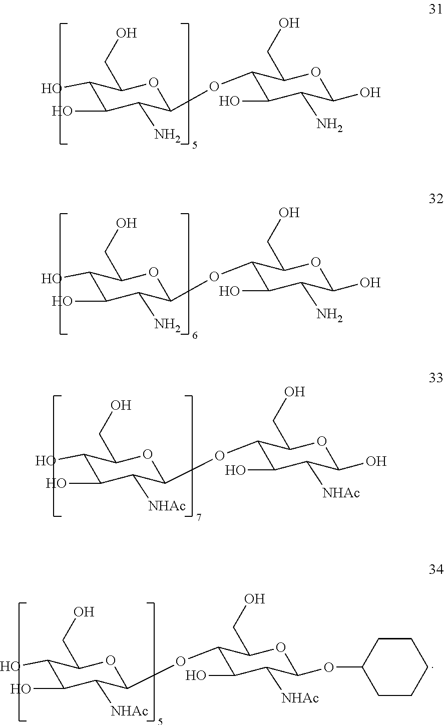

23. The adjuvant of claim 14, wherein the intermediate monocytes are HLA-DR⁻/CD163⁺.

24. The adjuvant of claim 14, wherein the compound does not bind to or trigger VEGF receptor.

25. The adjuvant of claim 14, wherein the compound binds peripheral blood mononuclear cells at both TLR4 and CD163.

26. The adjuvant of claim 14, wherein the compound decreases inflammatory cytokines in cord blood cells and CD8+ T cells in retinopathy of prematurity (ROP).

27. The adjuvant of claim 14, wherein the compound overcomes immune cell tolerance and primes immunity for prevention or treatment of bronchopulmonary dysplasia.

28. The adjuvant of claim 14, wherein the compound has at least one of: anti-inflammatory, anti-angiogenic, or anti-fibrotic activities.

29. A method for preventing or treating inflammatory diseases, conditions, or symptoms, the method comprising administering to a subject a prophylactically or therapeutically effective amount of a composition containing one or more pharmaceutically acceptable carriers and a compound of Formula I, or stereoisomer, enantiomer, tautomer or a pharmaceutically acceptable salt thereof: 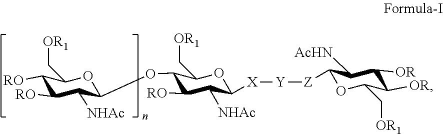
wherein n=0-5; X═NH, O, S, or CH₂; Y=Phenyl, a phenyl group substituted with at least one methyl, a phenyl group substituted with at least one nitro, a phenyl group substituted with at least one nitrogen, a phenyl group substituted with at least one boron, aryl, substituted aryl, heteroaryl, four to six membered cycloalkyl, four to six membered heterocycloalkyl; Z═NH, O, S, CH₂ or none; R═H, C(O)R₂, SO₂R₂; R₁═H, C(O)R₂, SO₂R₂; R₂=Ethyl, methyl, isopropyl, n-propyl, t-butyl, n-butyl, NH₂, NR₃R₄; R₃, R₄=ethyl, methyl, isopropyl, n-propyl, t-butyl, n-butyl, three to six membered cycloalkyl, wherein the compound promotes monocyte differentiation into an antigen-presenting cell (APC)-specific intermediate macrophage lineage.

30. The method of claim 29, the inflammatory disease, condition, or symptom is related to (a) decreased intermediate macrophages compared to normal condition and/or (b) decreased proportion and/or increased number of intermediate monocyte-derived macrophage compared to normal condition.

31. The method of claim 29 wherein the inflammatory disease, condition, or symptom is selected from the group consisting of single or multiple organ failure or dysfunction, bronchopulmonary dysplasia, retinopathy or prematurity, sepsis, cytokine storm, fever, neurological dysfunction or impairment, loss of taste or smell, cardiac dysfunction, pulmonary dysfunction, liver dysfunction, acute or chronic respiratory dysfunction, graft versus host disease (GVHD), cardiomyopathy, vasculitis, fibrosis, ophthalmic inflammation, dermatologic inflammation, gastrointestinal inflammation, tendinopathies, allergy, asthma, rheumatoid arthritis, glomerulonephritis, pancreatitis, hepatitis, non-alcoholic steatohepatitis (NASH), inflammatory arthritis, gout, multiple sclerosis, psoriasis, acute respiratory distress syndrome (ARDS), diabetic ulcers, non-healing wounds, nonalcoholic fatty liver disease (NAFLD), scleroderma, pulmonary arterial hypertension, scar tissues, atherosclerosis, vascular inflammation, neonatal hypoxia-ischemia brain injury, traumatic brain injury, ischemic stroke, hemorrhagic stroke, amyotrophic lateral sclerosis, neurodegenerative disease, lung infection, remote lung injury, chronic obstructive pulmonary disease, transfusion-induced lung injury, cisplatin-induced kidney injury, renal ischemia-reperfusion injury, renal transplantation, cardiac ischemia and infarction, cardiac transplantation, Crohn's and ulcerative colitis, terminal ileitis, alcoholic steatohepatitis, hepatotoxicity, liver infection, remote liver injury, lupus, autoimmune diseases associated with acute or chronic inflammation, and acute or chronic inflammation associated with viral, bacterial, or fungal infection.
